(19) United States
(12) Patent Application Publication
(10) Pub. No.: US 2022/0090217 A1
(43) Pub. Date: Mar. 24, 2022

(54) BIOMARKERS AND USES THEREOF IN THE TREATMENT OF CHRONIC HBV INFECTION

(71) Applicant: Janssen Sciences Ireland Unlimited Company, Co. Cork, IL (US)

(72) Inventors: Marianne Isabelle TUEFFERD, Berchem (Antwerp) (BE); Marjolein CRABBE, Kortrijk (BE); Jacques Armand Henri BOLLEKENS, Ixelles (BE); Jeroen Marcel Maria Roger AERSSENS, Nieuwrode (Holsbeek) (BE); Alessandro DI CARA, Beumont (FR)

(21) Appl. No.: 17/463,824

(22) Filed: Sep. 1, 2021

Related U.S. Application Data
(60) Provisional application No. 63/073,617, filed on Sep. 2, 2020.

(51) Int. Cl. C12Q 1/70; C12Q 1/6881

(52) U.S. Cl. CPC C12Q 1/706; C12Q 1/6881; C12Q 2600/158; C12Q 2600/156; C12Q 2600/106

(57) ABSTRACT

Immunogenetic biomarkers of viral control of chronic hepatitis B (CHB) infection that are indicative of relapse after a viral suppression treatment, such as a NUC treatment, in CHB infected subject are described. Also described are methods and compositions of using the immunogenetic biomarkers in predicting the relapse in the viral suppression treatment of CHB infection.

### CROSS REFERENCE TO RELATED APPLICATION

This application claims priority to U.S. Provisional Application No. 63/073,617 filed on Sep. 2, 2020, the disclosure of which is incorporated herein by reference in its entirety.

### FIELD OF THE INVENTION

The present invention is directed generally to immunogenetic biomarkers of viral control of chronic HBV infection (CHB) and related uses in the treatment of CHB.

### BACKGROUND OF THE INVENTION

Chronic infection caused by hepatitis B virus (HBV) affects about 400 million people worldwide and is among the world's leading causes of death. Association for the Study of Liver Disease (AASLD) guidelines recommend treatment for patients who present with levels of serum HBV DNA over 2,000 IU/mL and/or with elevated alanine aminotransferase (ALT) levels (>2 times upper limit of normal).

Antiviral therapy of chronic hepatitis B (CHB) is aimed to decrease the liver-related morbidity and mortality. The achievement of a sustained suppression of HBV replication has been associated with normalization of serum alanine transaminase (ALT), loss of hepatitis B e-antigen (HBeAg) with or without detection of anti-HBe, and improvement in liver histology. This goal can be achieved by, for example, short-term treatment with pegylated interferon (Peg-IFN) or long-term suppressive therapy with oral nucleotide or nucleoside analogues (NUCs) (Lok & McMahon, Hepatology, 2009, 50: 661-662; EASL clinical practice guidelines: management of chronic hepatitis B, J. Hepatol., 2012, 57:167-185). Recently, oral administration of NUCs has become the most popular treatment strategy worldwide given the excellent efficacy and safety of third-generation NUCs such as entecavir and tenofovir, not only in registration trials but also in clinical practice.

Oral anti-viral NUCs can be prescribed as once-daily oral dosing with minimal side effects, and are very effective in viral suppression and normalization of liver enzymes. However, most patients require long-term therapy and virological relapse is common after premature cessation of therapy (Ahn, et al., Hepatol. Int., 2010, 4: 386-95; van Nunen, et al., Gut., 2003, 52: 420-442).

Clearance of hepatitis B surface antigen (HBsAg) is the ideal endpoint to stop treatment, but its occurrence is usually lower than 5% in 5 years with anti-viral therapy. Recommendations about stopping treatment depend on different groups of CHB patients. Nonetheless, approximately 25% to 50% of the patients may still develop hepatitis relapse after stopping NUC therapy even if these recommendations are followed (Fung, et al., Am. J. Gastroenterol 2009; 104: 1940-6; Hadziyannis, et al., Hepatology 2006, 1: 231A).

Therefore, it would be desirable to be able to predict a patient's response to CHB therapy, and then accordingly adjust a treatment strategy right from the beginning, in order to minimize the risk of relapse after termination of treatment. In particular, it is preferred that the prediction is based on the detection of immunogenetic biomarkers of viral control of CHB.

The foregoing discussion is presented solely to provide a better understanding of the nature of the problems confronting the art and should not be construed in any way as an admission as to prior art, nor should the citation of any reference herein be construed as an admission that such reference constitutes “prior art” to the instant application.

### SUMMARY OF THE INVENTION

The present invention provides identification and uses of immunogenetic biomarkers of viral control of CHB in the treatment of CHB patients.

In one general aspect, the application relates to a method of determining whether a subject having a chronic hepatitis B (CHB) infection has a high or low probability of relapse after discontinuation of a viral suppression treatment, preferably a high or low probability of relapse within a period of two years after the discontinuation, the method comprising:
a. detecting in a biological sample obtained from the subject the presence of one or more immunogenetic biomarkers of viral control of the CHB infection, wherein the one or more immunogenetic biomarkers are selected from the group consisting of:
   i. a human leukocyte antigen (HLA) allele;
   ii. one or more HLA-C single nucleotide polymorphisms (SNPs);
   iii. a HLA evolutionary diversity (HED) score; and
   iv. leucocyte receptor complex (LCR) SNPs;
b. determining that the probability of relapse is low for the subject if the one or more immunogenetic biomarkers are detected in the biological sample, or determining that the probability of relapse is high for the subject if none of the immunogenetic biomarkers is detected in the biological sample.

In certain embodiments, the detection of the immunogenetic biomarkers is before, during, or after the viral suppression treatment.

In certain embodiments, the viral suppression treatment is a nucleotide or nucleoside (NUC) selected from the group consisting of tenofovir, entecavir, lamivudine, adefovir, and telbivudine.

In another general aspect, the application relates to a method of treating a chronic hepatitis B (CHB) infection in a subject in need thereof, the method comprising:
a. administering to the subject a viral suppression treatment to treat the CHB infection;
b. detecting in a biological sample obtained from the subject the presence of one or more immunogenetic biomarkers of viral control of the CHB infection, wherein the one or more immunogenetic biomarkers are selected from the group consisting of:
   i. a human leukocyte antigen (HLA) allele;
   ii. one or more HLA-C single nucleotide polymorphisms (SNPs);
   iii. a HLA evolutionary diversity (HED) score; and
   iv. leucocyte receptor complex (LCR) SNPs;
c. if the one or more immunogenetic biomarkers of step b are detected in the biological sample, discontinuing the viral suppression treatment once viral suppression is achieved, preferably the viral suppression treatment is discontinued after 2 years of treatment; or
   if none of the immunogenetic biomarkers of step b is detected in the biological sample, continuing the viral suppression treatment even after viral suppression is achieved, and/or administering to the subject a further or different viral suppression treatment.

In certain embodiments, the viral suppression treatment is a nucleotide or nucleoside (NUC) selected from the group consisting of tenofovir, entecavir, lamivudine, adefovir, and telbivudine.

In certain embodiments, the subject discontinues the viral suppression treatment when the subject achieves at least one of HBV DNA <60 IU/mL, ALT <80 U/L, and HBeAg negative.

In certain embodiments, the subject has no virological relapse or clinical relapse at or after 3 months, 6 months, 12 months, 18 months, 24 months, or 36 months after the discontinuation of the viral suppression treatment, or anytime in between, and wherein the virological relapse is identified as HBV DNA ≥2000 IU/ml or HBeAg positive, and the clinical relapse is identified as i) HBV DNA ≥2000 IU/ml or HBeAg positive, and ii) ALT ≥80 U/L.

In another general aspect, the application relates to a method of treating a chronic hepatitis B (CHB) infection in a subject in need thereof, the method comprising:
a. administering to the subject a viral suppression treatment to treat the CHB infection;
b. detecting in a biological sample obtained from the subject the presence of one or more immunogenetic biomarkers of viral control of the CHB infection, wherein the one or more immunogenetic biomarkers are selected from the group consisting of:
   i. a human leukocyte antigen (HLA) allele;
   ii. one or more HLA-C single nucleotide polymorphisms (SNPs);
   iii. a HLA evolutionary diversity (HED) score; and
   iv. leucocyte receptor complex (LCR) SNPs;
c. discontinuing the viral suppression treatment when the CHB infection is suppressed in the subject, and
d. administering to the subject the viral suppression treatment or another viral suppression treatment less than two years after the discontinuation if none of the immunogenetic biomarkers is detected in the biological sample.

In certain embodiments, the viral suppression treatment is a nucleotide or nucleoside (NUC) selected from the group consisting of tenofovir, entecavir, lamivudine, adefovir, and telbivudine.

In certain embodiments, the subject discontinues the viral suppression treatment when the subject achieves HBV DNA <60 IU/mL, ALT <80 U/L, or HBeAg negative.

In certain embodiments, the subject has no virological relapse or clinical relapse at or after 3 months, 6 months, 12 months, 18 months, 24 months, or 36 months after the discontinuation of the viral suppression treatment, or anytime in between, and wherein the virological relapse is identified as HBV DNA ≥2000 IU/ml or HBeAg positive, and the clinical relapse is identified as i) HBV DNA ≥2000 IU/ml or HBeAg positive, and ii) ALT ≥80 U/L.

In certain embodiments, the sample is selected from a tissue sample, a cellular sample, a blood sample. Preferably, the sample is a blood sample.

In certain embodiments, the immunogenetic biomarker comprises the presence of an HLA allele of B*51 or C*15, or the absence of an HLA allele C*07.

In certain embodiments, the immunogenetic biomarker comprises an HLA-C SNP selected from group consisting of rs2394952, rs3130542, rs2894202, rs9264523, rs1049281, rs9264643, rs1130838, rs2394888, AX-83089411, rs2308622, rs9264416, rs2001181, rs3132499, rs3130532, rs3130941, rs3130528, rs3134782, rs3134769, rs3130521, rs3130695, rs3130685, rs2524119, rs3130527, rs2894186, rs3130439, rs3095254, rs9264189, rs2394943, rs9394047, rs3130948, rs9368666, rs3130942, rs3130688, rs3130536, rs3134750, rs4084090, rs9264127, rs9264039, and rs3868078, or a complementary sequence thereof.

In certain embodiments, the immunogenetic biomarker comprises at least one HED score larger than the threshold value selected from the table below:

| HLA Regions | HLA gene | Variable | Threshold |
|---|---|---|---|
| Mean HLA Class I | HLA-A | HED_HLA_A_CAT_VR | 3.88 |
| Mean HLA Class I | HLA-B | HED_HLA_B_CAT_VR | 9.37 |
| Mean HLA Class I | HLA-C | HED_HLA_C_CAT_VR | 0.00 |
| Mean HLA Class I | MEAN_MHCI | HED_MEANI_CAT_VR | 8.69 |
| Mean HLA Class II | HLA-DPB1 | HED_HLA_P_CAT_VR | 2.21 |
| Mean HLA Class II | HLA-DQB1 | HED_HLA_Q_CAT_VR | 11.98 |
| Mean HLA Class II | HLA-DRB1 | HED_HLA_R_CAT_VR | 12.68 |
| Mean HLA Class II | MEAN_MHCII | HED_MEANII_CAT_VR | 7.55 |
| Mean HLA Class I | HLA-A | HED_HLA_A_CAT_CR | 3.88 |
| Mean HLA Class II | MEAN_MHCII | HED_MEANII_CAT_CR | 7.67 |
| Mean HLA Class I | HLA-C | HED_HLA_C_CAT_SCR | 7.37 |
| Mean HLA Class I | MEAN_MHCI | HED_MEANI_CAT_SCR | 7.61 |
| Mean HLA Class II | HLA-DRB1 | HED_HLA_R_CAT_SCR | 14.16 |
| Mean HLA Class II | MEAN_MHCII | HED_MEANII_CAT_SCR | 8.21 |

In certain embodiments, the immunogenetic biomarker comprises a LCR SNP selected from the group consisting of rs10426302, rs59537494, rs28366008, rs36625, rs635608, rs7595, rs731170, rs28513, rs12459334, rs11666535, rs4806807, rs11084367, rs39714, rs1654474, rs12462907, rs775893, rs10416527, rs4441391, rs40167, rs11084339, rs775875, rs2304225, rs4077076, rs4442924, rs4806527, rs12608979, rs3765013 of COSV52557220, rs12462181, rs2075731 of COSV52550169, rs12608988, rs190480734, rs1654452, rs11879415, rs653560, rs11084387, rs11672111, rs10424969, rs77389424, rs3745902 of CM1111041, rs11672983, rs17836364, rs34549987, rs11668526, rs11667105, rs1749282, rs1654660, rs73618328, rs270785, rs76522818, rs62131745, rs2241384, rs1325158, rs10500318, rs3816051, rs34508934, rs12460627, rs11667812, rs12974194, rs17836364, rs11669431, rs12984962, rs1761462, rs4806464, rs34190750, rs28681595, rs12610675, rs12463051, rs1749282, rs1654660, rs41275824, rs2241384, rs12983338, rs272408, rs10412569, AX-3232794851, rs622941, and rs60690598, or a complementary sequence thereof.

In another aspect, provided herein is a combination for predicting low risk of relapse after viral suppression in the treatment of a chronic hepatitis B (CHB) infection in a subject in need thereof, the combination comprising a reagent capable of detecting one or more of the immunogenetic biomarkers of viral control of CHB infection.

In yet another aspect, provided herein is a kit for serological HLA typing, or a kit for genetic HLA typing, such as a DNA chip, a PCR kit, or a set of PCR primers and/or probes, for use in predicting efficacy of a viral suppression agent in treating a chronic hepatitis B (CHB) infection in a subject in need thereof.

Further aspects, features and advantages of the present invention will be better appreciated upon a reading of the following detailed description of the invention and claims.

### BRIEF DESCRIPTION OF THE DRAWINGS

The foregoing summary, as well as the following detailed description of preferred embodiments of the present application, will be better understood when read in conjunction with the appended drawings. It should be understood, however, that the application is not limited to the precise embodiments shown in the drawings.

FIGS. 1A and 1B demonstrate cumulative incidence of virological relapse (FIG. 1A) and clinical relapse (FIG. 1B) after stop of treatment, which were assessed by Kaplan-Meier curves.

FIGS. 2A and 2B demonstrate the number of alleles observed at two digits resolution (FIG. 2a) and four digits resolution (FIG. 2b) for each HLA locus.

FIGS. 3A and 3B demonstrate the number of HLA homozygosity observed in the cohort for each allele at two digits resolution (FIG. 3a) and four digits resolution (FIG. 3b) for each HLA locus.

FIG. 4 represents a violin plot summarizing the distribution of HED scores (per locus) in the study cohort. Box plots are overlaid, representing the median HED score per locus (the limits of the boxes representing 25th and 75th percentiles and the whiskers respectively Q1 and Q3 plus 1.5 times inter-quartile range).

FIG. 5 shows FDR corrected p-values for HLA allelic association with clinical relapse (CR), virological relapse (VR), and sustained clinical response (SCR) for the additive (ADD), dominant (DOM) and homozygous (HOM) models. The dashed horizontal lines in the plot correspond to FDR 0.05 and 0.10.

FIGS. 6A-E demonstrate that HLA alleles (two digits resolution) are significantly associated with onset of virological relapse and/or sustained clinical response. As indicated in FIGS. 6A-6C, Kaplan-Meier curves of B*51 (A), C*07 (B) and C*15 (C) alleles are associated with onset of virological relapse, and barplots represent the distribution of presence and absence of B*51 alleles (A) (FIG. 6A) and C*15 alleles (C) (FIG. 6C) within sustained clinical responders. As indicated in FIG. 6D, single nucleotide polymorphisms (SNPs) in the HLA-C region showed similar level of significance (p-value<1e-6) while testing association with onset of virological or clinical relapse as a candidate approach. The plot of p-values is relative to genomic positions from the GWAS association analysis on onset of clinical relapse (top panel) or onset of viral relapse (2nd panel). Each point corresponds to the p-value of a SNP. The labels indicate the RS identifiers for the SNP corresponding to the lowest p-value obtained. As indicated in FIG. 6E, SNPs in the leukocyte receptor complex (LRC region is defined in (GRCh37.p13): CHR 19 54528888-55595686) including KIR ligands are associated with the onset of virological and clinical relapse. The plot of p-values is relative to genomic positions from the GWAS association analysis on onset of clinical relapse (top panel) or onset of viral relapse (2nd panel). Each point corresponds to the p-value of a SNP. The labels indicate the RS identifiers for the SNP corresponding to the 10 lowest p-values. The middle panel shows the linkage disequilibrium between pairs of SNPs using the r2 as metric. The bottom panel provides an overview of the the genes contained in the genomic region 19:54528888-55595686. The genes span the region as well as the coverage of the SNPs on the axiom chip (bottom track) and the position on the chromosome.

FIG. 7 demonstrates that HLA evolutionary diversity is significantly associated with onset of virological relapse, onset of clinical relapse and sustained clinical response. Kaplan-Meier curves representing HED scores for HLA class I and class II associated with onset of virological relapse are shown in FIGS. 7A and 7B. Barplots representing the distribution association of high HED score with sustained clinical responders are added to FIGS. 7A and 7B (mean class I and mean class II) and FIG. 7C (HLA-C and HLA-DRB1). Kaplan-Meier curves representing HED scores for HLA class II and HLA-A associated with onset of clinical relapse are shown in FIG. 7D.

FIG. 8 demonstrates Cox proportional hazard regression analysis for clinical relapse including HLA HED scores for HLA-A region and HLA-class II region. For each variable, the estimated value of the hazard ratio (above/below 1) indicates the negative/positive association to relapse.

FIG. 9 demonstrates Cox proportional hazard regression analysis for virological relapse including HLA B*51, HLA alleles C*07, HED scores in HLA class I and class II region. For each variable, the estimated value of the hazard ratio (above/below 1) indicates the negative/positive association to relapse. FIG. 10 demonstrates logistic regression analysis for sustained clinical response including HLA alleles B*51, HLA alleles C*51, HED scores in HLA class I and class II region. For each variable, the estimated value of the odds ratio (below/above 1) indicates the negative/positive association to sustained clinical response.

### DETAILED DESCRIPTION OF THE INVENTION

Various publications, articles and patents are cited or described in the background and throughout the specification; each of these references is herein incorporated by reference in its entirety. Discussion of documents, acts, materials, devices, articles or the like which has been included in the present specification is for the purpose of providing context for the invention. Such discussion is not an admission that any or all of these matters form part of the prior art with respect to any inventions disclosed or claimed.

Unless defined otherwise, all technical and scientific terms used herein have the same meaning as commonly understood to one of ordinary skill in the art to which this invention pertains. Otherwise, certain terms used herein have the meanings as set forth in the specification.

It must be noted that as used herein and in the appended claims, the singular forms “a,” “an,” and “the” include plural reference unless the context clearly dictates otherwise.

Unless otherwise stated, any numerical values, such as a concentration or a concentration range described herein, are to be understood as being modified in all instances by the term “about.” Thus, a numerical value typically includes ±10% of the recited value. For example, a concentration of 1 mg/mL includes 0.9 mg/mL to 1.1 mg/mL. Likewise, a concentration range of 1% to 10% (w/v) includes 0.9% (w/v) to 11% (w/v). As used herein, the use of a numerical range expressly includes all possible subranges, all individual numerical values within that range, including integers within such ranges and fractions of the values unless the context clearly indicates otherwise.

Unless otherwise indicated, the term “at least” preceding a series of elements is to be understood to refer to every element in the series. Those skilled in the art will recognize, or be able to ascertain using no more than routine experimentation, many equivalents to the specific embodiments of the invention described herein. Such equivalents are intended to be encompassed by the invention.

As used herein, the terms “comprises,” “comprising,” “includes,” “including,” “has,” “having,” “contains” or “containing,” or any other variation thereof, will be understood to imply the inclusion of a stated integer or group of integers but not the exclusion of any other integer or group of integers and are intended to be non-exclusive or open-ended. For example, a composition, a mixture, a process, a method, an article, or an apparatus that comprises a list of elements is not necessarily limited to only those elements but can include other elements not expressly listed or inherent to such composition, mixture, process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

It should also be understood that the terms “about,” “approximately,” “generally,” “substantially” and like terms, used herein when referring to a dimension or characteristic of a component of the preferred invention, indicate that the described dimension/characteristic is not a strict boundary or parameter and does not exclude minor variations therefrom that are functionally the same or similar, as would be understood by one having ordinary skill in the art. At a minimum, such references that include a numerical parameter would include variations that, using mathematical and industrial principles accepted in the art (e.g., rounding, measurement or other systematic errors, manufacturing tolerances, etc.), would not vary the least significant digit.

The term “immunogenetic biomarker” or “biomarker” as used herein refers generally to a molecule, including a gene, protein, carbohydrate structure, or glycolipid, the expression of which in or on a mammalian tissue or cell or secreted can be detected by known methods (or methods disclosed herein) or a property of one or more of such molecules, and is predictive or can be used to predict (or aid prediction) for a mammalian cell's or tissue's sensitivity to, and in some embodiments, to predict (or aid prediction) an individual's responsiveness to treatment regimens. In some embodiment, a biomarker includes the functional diversity score of genes, such as HLA evolutionary diversity (HED) score. The immunogenetic biomarkers are associated with the protection against onset of virological/clinical relapse and/or sustained clinical response after a viral suppression treatment. The biomarkers disclosed herein are genes, proteins, and/or scores, whose presence correlates with the absence of relapse or sustained clinical response after discontinuation of a viral suppression treatment such as NUC treatment of a liver disease (e.g., chronic hepatitis B infection).

As used herein, “human leukocyte antigen” or “HLA” refers to a group of related proteins that are encoded by the major histocompatibility complex (MHC) gene complex in humans. HLAs corresponding to MHC class I (A, B, and C) present peptides from inside the cell. These peptides are produced from digested proteins that are broken down in the proteasomes. Foreign antigens presented by MHC class I attract cytotoxic T-cells that destroy cells. HLAs corresponding to MHC class II (DP, DM, DO, DQ, and DR) present antigens from outside of the cell to T-lymphocytes. Extracellular proteins are endocytosed, digested in lysosomes, and the resulting epitopic peptide fragments are loaded onto MHC class II molecules prior to their migration to the cell surface. These particular antigens stimulate the multiplication of T-helper cells, which in turn stimulate antibody-producing B-cells to produce antibodies to that specific antigen.

As used herein, “HLA evolutionary diversity score,” “HLA evolutionary divergence score” or “HED score” is intended in accordance with its ordinary meaning, e.g., as described in Pierini and Lenz, Mol. Biol., Evol. 2018, 35(9):2145-2158. It refers to a measure taking into account the predicted influence on amino-acid composition (estimated with by the Grantham distance), which has been derived for each patient as a mean HED across all HLA class I and class II genes separately per gene HED score is quantitative, and can be computed for each individual across or within HLA regions.

As used herein, “probe” refers to any molecule or agent that is capable of selectively binding to an intended target biomolecule. The target molecule can be a biomarker, for example, a nucleotide transcript or a protein encoded by or corresponding to a biomarker. Probes can be synthesized by one of skill in the art, or derived from appropriate biological preparations, in view of the present disclosure. Probes can be specifically designed to be labeled. Examples of molecules that can be utilized as probes include, but are not limited to, RNA, DNA, proteins, peptides, antibodies, aptamers, affibodies, and organic molecules.

As used herein, a “baseline gene expression” of a gene in a subject refers to the gene expression level of the gene in the subject before the subject is treated for the liver diseases.

As used herein, “subject” means any animal, preferably a mammal, most preferably a human. The term “mammal” as used herein, encompasses any mammal. Examples of mammals include, but are not limited to, cows, horses, sheep, pigs, cats, dogs, mice, rats, rabbits, guinea pigs, monkeys, humans, etc., more preferably a human.

As used herein, “sample” is intended to include any sampling of cells, tissues, or bodily fluids in which expression of a biomarker can be detected. Examples of such samples include, but are not limited to, biopsies, smears, blood, lymph, urine, saliva, or any other bodily secretion or derivative thereof. Blood can, for example, include whole blood, plasma, serum, or any derivative of blood. Samples can be obtained from a subject by a variety of techniques, which are known to those skilled in the art.

As used herein, “treatment” refers to both therapeutic treatment and prophylactic or preventative measures, wherein the object is to prevent or slow down (lessen) the targeted pathologic condition or disorder. Those in need of treatment include those diagnosed with the disorder as well as those prone to have the disorder (e.g., a genetic predisposition) or those in whom the disorder is to be prevented.

A “single nucleotide polymorphism”, or “SNP”, refers to a single base position in an RNA or DNA molecule (e.g., a polynucleotide), at which different alleles, or alternative nucleotides, exist in a population. The SNP position (interchangeably referred to herein as SNP, SNP site, SNP locus) is usually preceded by and followed by highly conserved sequences of the allele (e.g., sequences that vary in less than 1/100 or 1/1000 members of the populations). An individual can be homozygous or heterozygous for an allele at each SNP position.

Where there are two, three, or four alternative nucleotide sequences at a polymorphic locus, each nucleotide sequence is referred to as a “polymorphic variant” or “nucleic acid variant.” Where two polymorphic variants exist, for example, the polymorphic variant represented in a minority of samples from a population is sometimes referred to as a “minor allele” and the polymorphic variant that is more prevalently represented is sometimes referred to as a “major allele.” Many organisms possess a copy of each chromosome (e.g., humans), and those individuals who possess two major alleles or two minor alleles are often referred to as being “homozygous” with respect to the polymorphism, and those individuals who possess one major allele and one minor allele are normally referred to as being “heterozygous” with respect to the polymorphism. Individuals who are homozygous with respect to one allele are sometimes predisposed to a different phenotype as compared to individuals who are heterozygous or homozygous with respect to another allele.

In genetic analysis that identifies one or more immunogenetic biomarkers, samples from individuals having different values in a relevant phenotype often are allelotyped and/or genotyped. The term “allelotype” as used herein refers to a process for determining the allele frequency for a polymorphic variant in pooled DNA samples from cases and controls. By pooling DNA from each group, an allele frequency for each locus in each group is calculated. These allele frequencies are then compared to one another.

The term “linkage disequilibrium” or “LD” refers to the co-inheritance of alleles (e.g., alternative nucleotides) at two or more different SNP sites at frequencies greater than would be expected from the separate frequencies of occurrence of each allele in a given population. The expected frequency of co-occurrence of two alleles that are inherited independently is the frequency of the first allele multiplied by the frequency of the second allele. Alleles that co-occur at expected frequencies are said to be in “linkage equilibrium”. In contrast, LD refers to any non-random genetic association between allele(s) at two or more different SNP sites, which is generally due to the physical proximity of the two loci along a chromosome. See e.g., U.S. 2008/0299125.

In some embodiments, LD can occur when two or more SNPs sites are in close physical proximity to each other on a given chromosome and therefore alleles at these SNP sites will tend to remain unseparated for multiple generations with the consequence that a particular nucleotide (allele) at one SNP site will show a non-random association with a particular nucleotide (allele) at a different SNP site located nearby. Hence, genotyping one of the SNP sites will give almost the same information as genotyping the other SNP site that is in LD. See e.g., U.S. 2008/0299125.

In some embodiments, for diagnostic purposes, if a particular SNP site is found to be useful for diagnosing, then the skilled artisan would recognize that other SNP sites which are in LD with this SNP site would also be useful for diagnosing the condition. Various degrees of LD can be encountered between two or more SNPs with the result being that some SNPs are more closely associated (i.e., in stronger LD) than others.

Furthermore, the physical distance over which LD extends along a chromosome differs between different regions of the genome, and therefore the degree of physical separation between two or more SNP sites necessary for to occur can differ between different regions of the genome. See e.g., U.S. 2008/0299125.

A genotype or polymorphic variant may be expressed in terms of a “haplotype,” which as used herein refers to a set of DNA variations, or polymorphisms, that tend to be inherited together. A haplotype can refer to a combination of alleles or to a set of SNPs found on the same chromosome. For example, two SNPs may exist within a gene where each SNP position includes a cytosine variation and an adenine variation. Certain individuals in a population may carry one allele (heterozygous) or two alleles (homozygous) having the gene with a cytosine at each SNP position. As the two cytosines corresponding to each SNP in the gene travel together on one or both alleles in these individuals, the individuals can be characterized as having a cytosine/cytosine haplotype with respect to the two SNPs in the gene.

The term “amino acid variation” refers to a change in an amino acid sequence (e.g., an insertion, substitution, or deletion of one or more amino acids, such as an internal deletion or an N- or C-terminal truncation) relative to a reference sequence.

The term “variation” refers to either a nucleotide variation or an amino acid variation.

The term “array or microarray” refers to an ordered arrangement of hybridizable array elements, preferably polynucleotide probes e.g., oligonucleotides), on a substrate. The substrate can be a solid substrate, such as a glass slide, or a semi-solid substrate, such as nitrocellulose membrane.

The term “administering” with respect to the methods of the invention, means a method for therapeutically or prophylactically preventing, treating or ameliorating a syndrome, disorder or disease as described herein. Such methods include administering an effective amount of said therapeutic agent at different times during the course of a therapy or concurrently in a combination form. The methods of the invention are to be understood as embracing all known therapeutic treatment regimens.

The term “effective amount” means that amount of active compound or pharmaceutical agent that elicits the biological or medicinal response in a tissue system, animal or human, that is being sought by a researcher, veterinarian, medical doctor, or other clinician, which includes preventing, treating or ameliorating a syndrome, disorder, or disease being treated, or the symptoms of a syndrome, disorder or disease being treated (e.g., CHB).

The term “viral suppression agent” or “viral suppression treatment” is in accordance with its ordinary meaning in the field and includes any agent which suppresses the activities of virus, more particularly hepatitis B virus (HBV). Examples of viral suppression agents include, but are not limited to, nucleotide or nucleoside analogues (NUCs) such as entecavir (Baraclude), tenofovir (Viread), lamivudine (Epivir), adefovir (Hepsera) and telbivudine (Tyzeka).

The term “non-NUC treatment” encompasses a treatment using non-NUC agents, a treatment using a combination of a NUC agent and other agent, as well as stopping treatment.

Treatment of CHB

Clearance of HBsAg, with seroconversion to HBs antibodies (anti-HBs) is the closest correlate of cure and the ultimate goal of CHB therapy. CHB treatments include immunomodulators and viral suppression agents such as NUCs. Examples of immunomodulators include, but are not limited to, IFN-α, Peg-IFN-α, thymosin-α1 and oxymatrine. Interferons (IFNs) are cytokines which interfere with viral replication in host cells by inhibiting viral DNA synthesis, and enhancing the cellular immune response against HBV-infected hepatocytes. In general, interferon (IFN) therapy has a finite duration of treatment and is more likely to produce a sustained virological response. Its use, however, is limited by high costs and numerous associated side effects.

Examples of NUCs include, but are not limited to tenofovir, entecavir, lamivudine, adefovir, and telbivudine. The goal of NUC therapy (NA) for chronic hepatitis B (CHB) is to suppress hepatitis B virus (HBV) replication in a sustained manner, preventing disease progression to decompensated cirrhosis and hepatocellular carcinoma (HCC). Overall, all NAs have an excellent safety profile across a wide spectrum of persons with CHB, including those with decompensated cirrhosis and transplant recipients. Indeed, more than 90% of patients with CHB are currently treated with oral NUCs worldwide. However, the NUCs tend not to eradicate HBV as they do not impact HBV cccDNA, which acts as an ongoing source of viral persistence, therefore, long-term treatment with NUCs is required to maintain virological control.

To avoid lifelong NUC treatment, new strategies are being assessed in clinical trials, including switching to or adding on Peg-IFN, combination with oral immunomodulatory agents, and discontinuation in selected HBeAg-negative patients according to HBsAg levels.

Relapses After Viral Suppression Treatment of CHB

The duration of viral suppression treatment requires that at least once complete virological suppression is achieved. Although loss of HBsAg is the ideal endpoint associated with sustained off-treatment virological suppression, HBsAg is only cleared in a minority of CHB patients after antiviral therapy. HBeAg loss and/or seroconversion has been widely used as a surrogate endpoint of CHB therapy, and several practice guidelines suggest that HBV DCC such as NUC treatment may be stopped when the patient achieves HBV DNA <1000 IU/mL, more particularly HBV DNA <60 IU/mL, more particularly HBV DNA <1000 IU/mL and ALT <80 IU/L, more particularly HBV DNA <60 IU/mL and ALT <80 IU/L, more particularly HBV DNA <1000 IU/mL and serum HBsAg <100 IU/mL, more particularly HBV DNA <60 IU/mL and HBsAg <100 IU/mL. Nonetheless, approximately 25% to 50% of the patients may still develop relapse after stopping anti-viral therapy even if these recommendations are followed.

Hepatitis relapses involve transient abnormalities in the alanine aminotransferase (ALT) level or the HBV DNA level, as well as HBeAg level. Hepatitis relapses can be characterized as virological relapse, biomedical relapse, or clinical relapse. Currently, using the same limits as the guidelines for the initiation of therapy, an HBV DNA level ≥2000 IU/mL or HBeAg positive can be considered as virological relapse. Biochemical relapse is defined as an elevation of ALT levels >1 time (1×), 1.5× or 2× the upper limit of normal (ULN) depending on study criteria. The current upper limit of serum ALT, though varied among laboratories, is generally around 40 IU/L. In some studies, the term clinical relapse is used, which considers group both virological and biochemical relapses. In some studies, a patient is denoted a sustained clinical responder in case no clinical and no virological relapse occurred during the entire follow-up period after treatment cessation.

Immunogenetic Biomarker for Predicting a Relapse or a Sustained Clinical Response After Viral Suppression Treatment of CHB

The present invention relates generally to the prediction of a relapse or a sustained clinical response in a subject diagnosed with CHB after treatment, and provides methods, reagents, and kits useful for this purpose. Provided herein are immunogenetic biomarkers that are indicative of and/or predictive for a relapse or a sustained clinical response after the treatment. In certain embodiments, the treatment is a viral suppression treatment such as a NUC treatment. In certain embodiments, the prediction of the relapse or the sustained clinical response is performed before, during, or after the treatment of CHB.

In certain embodiments, the present invention provides immunogenetic biomarkers of viral control of CHB infection for predicting a relapse after the discontinuation of a viral suppression treatment in a subject diagnosed with CHB. According to the embodiments of this invention, the presence of one or more of the immunogenetic biomarkers is negatively associated with the incidence of such relapse.

In certain embodiments, the present invention provides immunogenetic biomarkers of viral control of CHB infection for predicting a sustained clinical response after the discontinuation of viral suppression treatment in a subject diagnosed with CHB. According to the embodiments of this invention, the presence of one or more of the immunogenetic biomarkers is positively associated with the incidence of such sustained clinical response.

Also provided are kits, chips, devices, or assays for use in accordance with the present invention. Such an assay, chip, device, or a kit can comprise a plurality of primers or probes to detect one or more of the immunogenetic biomarkers of viral control of CHB infection described herein. Such kits, chips, devices can include instruments and instructions that a subject can use to obtain a sample, e.g., of buccal cells or blood, without the aid of a health care provider.

In some embodiments, the invention provides compositions and kits comprising primers and primer pairs, which allow the specific amplification of the polynucleotides of the invention or of any specific parts thereof, and probes that selectively or specifically hybridize to nucleic acid molecules of the invention or to any part thereof. Probes can be labeled with a detectable marker, such as, for example, a radioisotope, fluorescent compound, bioluminescent compound, a chemiluminescent compound, metal chelator or enzyme. Such probes and primers can be used to detect the presence of polynucleotides in a sample and as a means for detecting cell expressing proteins encoded by the polynucleotides. As will be understood by the skilled artisan, a great many different primers and probes can be prepared based on the sequences provided herein and used effectively to amplify, clone and/or determine the presence of an immunogenetic biomarker of interest.

The application also contemplates the development of computer algorithm which will convert the test results generated from the measurement of the immunogenetic biomarkers into an output, e.g., a score, which will be used to determine in whether an individual is likely to have a relapse or a sustained clinical response after a treatment of CHB.

In one general aspect, provided is a panel of immunogenetic biomarkers of viral control of CHB infection for determining whether a subject having a chronic hepatitis B (CHB) infection has a high or low probability of relapse after discontinuation of a viral suppression treatment.

According to the invention, the panel of the immunogenetic biomarkers is able to identify subsets of patients with different risk to hepatitis relapses after treatment of CHB, which could be beneficial in many ways, including reduced exposure of patients to ineffective treatments, achievement of higher response rates, and the ability to treat predicted patients with alternative therapies to avoid or minimize possible relapse. The biomarkers can additionally be used for other purposes, such as to stratify patients in clinical trials, reduce sample size in proof of concept trials by excluding subpopulations, and balance treatment arms in clinical trials.

Preferably, the immunogenetic biomarkers are selected from the group consisting of:
i. a human leukocyte antigen (HLA) allele;
ii. one or more HLA-C single nucleotide polymorphisms (SNPs);
iii. a HLA evolutionary diversity (HED) score; and
iv. leucocyte receptor complex (LCR) SNPs;

The panel of the immunogenetic biomarkers can contain 1, 2, 3, or 4 types of the immunogenetic biomarkers described herein. Preferably, the panel of the immunogenetic biomarkers is used to determine whether a subject having a chronic hepatitis B (CHB) infection has a high or low probability of relapse within a period of two years after the discontinuation of the viral suppression treatment.

In certain embodiments, the prediction of the probability of relapse is performed before, during, or after the viral suppression treatment of CHB.

In certain embodiments, the immunogenetic biomarker comprises the presence of an HLA allele of B*51 or C*15, or the absence of an HLA allele C*07. The presence of an HLA allele of B*51 or C*15, or the absence of an HLA allele C*07 is predictive of a low probability of relapse after the discontinuation of the viral suppression treatment, preferably a low probability of a virological relapse. The absence of an HLA allele of B*51 or C*15, or the presence of an HLA allele C*07 is predictive of a high probability of relapse after the discontinuation of the viral suppression treatment, preferably a low probability of a virological relapse. In further embodiments, the HLA allele of C*15 is C*15:02 and the HLA allele C*07 is C*07:02.

In certain embodiments, the immunogenetic biomarker comprises an HLA-C SNP selected from the group consisting of rs2394952, rs3130542, rs2894202, rs9264523, rs1049281, rs9264643, rs1130838, rs2394888, AX-83089411, rs2308622, rs9264416, rs2001181, rs3132499, rs3130532, rs3130941, rs3130528, rs3134782, rs3134769, rs3130521, rs3130695, rs3130685, rs2524119, rs3130527, rs2894186, rs3130439, rs3095254, rs9264189, rs2394943, rs9394047, rs3130948, rs9368666, rs3130942, rs3130688, rs3130536, rs3134750, rs4084090, rs9264127, rs9264039, and rs3868078, or a complementary sequence thereof. The presence of the HLA-C SNP described herein is predictive of a low probability of relapse after the discontinuation of the viral suppression treatment. The absence of the HLA-C SNP described herein is predictive of a high probability of relapse after the discontinuation of the viral suppression treatment.

In further embodiments, the HLA-C SNP is selected from the group consisting of rs2394952, rs3130542, rs2894202, rs9264523, rs1049281, rs9264643, rs1130838, rs2394888, AX-83089411, rs2308622, rs9264416, rs2001181, rs3132499, rs3130532, rs3130941, rs3130528, rs3134782, rs3134769, rs3130521, rs3130695, rs3130685, rs2524119, rs3130527, rs2894186, rs3130439, rs3095254, rs9264189, rs2394943, rs9394047, rs3130948, rs9368666, rs3130942, rs3130688, rs3130536, and rs3134750, or a complementary sequence thereof. The HLC-C SNPs described herein can be used to predict the probability of a virological relapse after the discontinuation of the viral suppression treatment of CHB.

In further embodiments, the HLA-C SNP is selected from the group consisting of rs4084090, rs9264127, rs9264039, and rs3868078, or a complementary sequence thereof. The HLC-C SNPs described herein can be used to predict the probability of a clinical relapse after the discontinuation of the viral suppression treatment of CHB.

In certain embodiments, the immunogenetic biomarker comprises at least one HED score larger than the threshold value selected from the table below:

| HLA Regions | HLA gene | Variable | Threshold |
|---|---|---|---|
| Mean HLA Class I | HLA-A | HED_HLA_A_CAT_VR | 3.88 |
| Mean HLA Class I | HLA-B | HED_HLA_B_CAT_VR | 9.37 |
| Mean HLA Class I | HLA-C | HED_HLA_C_CAT_VR | 0.00 |
| Mean HLA Class I | MEAN_MHCI | HED_MEANI_CAT_VR | 8.69 |
| Mean HLA Class II | HLA-DPB1 | HED_HLA_P_CAT_VR | 2.21 |
| Mean HLA Class II | HLA-DQB1 | HED_HLA_Q_CAT_VR | 11.98 |
| Mean HLA Class II | HLA-DRB1 | HED_HLA_R_CAT_VR | 12.68 |
| Mean HLA Class II | MEAN_MHCII | HED_MEANII_CAT_VR | 7.55 |
| Mean HLA Class I | HLA-A | HED_HLA_A_CAT_CR | 3.88 |
| Mean HLA Class II | MEAN_MHCII | HED_MEANII_CAT_CR | 7.67 |
| Mean HLA Class I | HLA-C | HED_HLA_C_CAT_SCR | 7.37 |
| Mean HLA Class I | MEAN_MHCI | HED_MEANI_CAT_SCR | 7.61 |
| Mean HLA Class II | HLA-DRB1 | HED_HLA_R_CAT_SCR | 14.16 |
| Mean HLA Class II | MEAN_MHCII | HED_MEANII_CAT_SCR | 8.21 |

The presence of the HED score described herein is predictive of a low probability of relapse after the discontinuation of the viral suppression treatment. The absence of the HED score described herein is predictive of a high probability of relapse after the discontinuation of the viral suppression treatment.

In further embodiments, the HED score is selected from the group consisting of a value of HED HLA-A larger than 3.88, a value of HED HLA-B larger than 9.37, a value of HED HLA-C larger than 0.00, a value of mean HED HLA Class I larger than 8.69, a value of HED HLA-DPB1 larger than 2.21, a value of HED HLA-DQB1 larger than 11.98, a value of HED HLA-DRB1 larger than 12.68, and a value of mean HED HLA Class II larger than 7.55. The HED scores described herein can be used to predict the probability of a virological relapse after the discontinuation of the viral suppression treatment of CHB.

In further embodiments, the HED score is selected from the group consisting of a value of HED HLA-A larger than 3.88, and a value of mean HED HLA Class II larger than 7.67. The HED scores described herein can be used to predict the probability of a clinical relapse after the discontinuation of the viral suppression treatment of CHB.

In further embodiments, the HED score is selected from the group consisting of a value of HED HLA-C larger than 7.37, a value of mean HED HLA Class I larger than 7.61, a value of HED HLA-DRB1 larger than 14.16, and a value of mean HED HLA Class II larger than 8.21. The HED scores described herein can be used to predict the probability of a sustained clinical response after the discontinuation of the viral suppression treatment of CHB.

In certain embodiments, the immunogenetic biomarker comprises a LCR SNP selected from group consisting of rs10426302, rs59537494, rs28366008, rs36625, rs635608, rs7595, rs731170, rs28513, rs12459334, rs11666535, rs4806807, rs11084367, rs39714, rs1654474, rs12462907, rs775893, rs10416527, rs4441391, rs40167, rs11084339, rs775875, rs2304225, rs4077076, rs4442924, rs4806527, rs12608979, rs3765013 of COSV52557220, rs12462181, rs2075731 of COSV52550169, rs12608988, rs190480734, rs1654452, rs11879415, rs653560, rs11084387, rs11672111, rs10424969, rs77389424, rs3745902 of CM1111041, rs11672983, rs17836364, rs34549987, rs11668526, rs11667105, rs1749282, rs1654660, rs73618328, rs270785, rs76522818, rs62131745, rs2241384, rs1325158, rs10500318, rs3816051, rs34508934, rs12460627, rs11667812, rs12974194, rs17836364, rs11669431, rs12984962, rs1761462, rs4806464, rs34190750, rs28681595, rs12610675, rs12463051, rs1749282, rs1654660, rs41275824, rs2241384, rs12983338, rs272408, rs10412569, AX-3232794851, rs622941, and rs60690598, or a complementary sequence thereof. The presence of the LCR SNP described herein is predictive of a low probability of relapse after the discontinuation of the viral suppression treatment. The absence of the LCR SNP described herein is predictive of a high probability of relapse after the discontinuation of the viral suppression treatment.

In further embodiments, the LCR SNP is selected from the group consisting of rs10426302, rs59537494, rs28366008, rs36625, rs635608, rs7595, rs731170, rs28513, rs12459334, rs11666535, rs4806807, rs11084367, rs39714, rs1654474, rs12462907, rs775893, rs10416527, rs4441391, rs40167, rs11084339, rs775875, rs2304225, rs4077076, rs4442924, rs4806527, rs12608979, rs3765013 of COSV52557220, rs12462181, rs2075731 of COSV52550169, rs12608988, rs190480734, rs1654452, rs11879415, rs653560, rs11084387, rs11672111, rs10424969, rs77389424, rs3745902 of CM1111041, rs11672983, rs17836364, rs34549987, rs11668526, rs11667105, rs1749282, rs1654660, rs73618328, rs270785, rs76522818, rs62131745, rs2241384, rs1325158, rs10500318, rs3816051, rs34508934, and rs12460627, or a complementary sequence thereof. The LCR SNPs described herein can be used to predict the probability of a virological relapse after the discontinuation of the viral suppression treatment of CHB.

In further embodiments, the LCR SNP is selected from the group consisting of rs11667812, rs12974194, rs17836364, rs11669431, rs12984962, rs1761462, rs4806464, rs34190750, rs28681595, rs12610675, rs12463051, rs1749282, rs1654660, rs41275824, rs2241384, rs12983338, rs272408, rs10412569, AX-3232794851, rs622941, and rs60690598, or a complementary sequence thereof. The LCR SNPs described herein can be used to predict the probability of a clinical relapse after the discontinuation of the viral suppression treatment of CHB.

In certain embodiments, the immunogenetic biomarkers are determined by Genome-wide genotyping (GMAS), wherein the genotype calling is performed on biallelic SNPs. In certain embodiments, the immunogenetic biomarkers are determined by Human leukocyte antigen (HLA) typing by Sanger sequencing, or by polymerase chain reaction-sequence-based-typing (PCR-SBT) methodology.

In certain embodiments, the SNP or the allele is determined by a method selected from the group consisting of DNA sequencing, restriction fragment length polymorphism (RFLP analysis), allele specific oligonucleotide (ASO) analysis, Denaturing/Temperature Gradient Gel Electrophoresis (DGGE/TGGE), Single-Strand Conformation Polymorphism (SSCP) analysis, Dideoxy fingerprinting (ddF), pyrosequencing analysis, acycloprime analysis, Reverse dot blot, GeneChip microarrays, Dynamic allele-specific hybridization (DASH), Peptide nucleic acid (PNA) and locked nucleic acids (LNA) probes, TaqMan, Molecular Beacons, Intercalating dye, FRET primers, AlphaScreen, SNPstream, genetic bit analysis (GBA), Multiplex minisequencing, SNaPshot, MassEXTEND, MassArray, GOOD assay, Microarray miniseq, arrayed primer extension (APEX), Microarray primer extension, Tag arrays, Coded microspheres, Template-directed incorporation (TDI), fluorescence polarization, Colorimetric oligonucleotide ligation assay (OLA), Sequence-coded OLA, Microarray ligation, Ligase chain reaction, Padlock probes, Rolling circle amplification, and Invader assay.

According to the embodiments of the application, the HED score is determined by any known method known to those skilled in the art. The HED score is typically measured taking into account the predicted influence of amino acid composition and can be derived across all genes separately. The 4-field HLA genetic codes can then be used to match the alleles to the corresponding proteins in the alignment files. For each pair of alleles in the genotypes their functional distance in terms of proteins encoded is measured using the Grantham distance metric. For each HLA protein locus, amino-acid co-ordinates of the MHC antigen-recognition domains can be identified using the Ensembl database protein annotations (ftp://ftp.ebi.ac.uk/pub/databases/ipd/imgt/hla/alignments/; Robinson, et al., Nucleic acids research, 2020, 48: D948-55; Zerbino, et al., Nucleic Acids Research, 2018, 46: D754-61). Globally the co-ordinates cover exons 2 and 3 in HLA-type I genes and exon 2 and a portion of exon 1 in HLA-type II genes. For each subject and HLA locus, the HLA genotypes using a 4-field HLA allelic identification code are extracted. The 4-field HLA genetic codes are then used to match the alleles to the corresponding proteins in the alignment files. For each pair of alleles in the genotypes their functional distance in terms of proteins encoded is measured using the Grantham distance metric. The Grantham distance is a quantitative pairwise distance between two amino-acids. The distance for a pair takes into account their physiochemical properties (biochemical composition, polarity and volume of each amino-acid). The Grantham overall divergence for a given alignment of a pair of sequences is calculated by summing the individual amino-acid pair distance value and then by dividing the sum by the alignment length (Grantham, et al., Science, 1974, 185: 862-4).

In certain embodiments, an array of the application comprises individual or collections of nucleic acid molecules useful for detecting SNPs described herein. For instance, an array of the application can comprise a series of discretely placed individual nucleic acid oligonucleotides or sets of nucleic acid oligonucleotide combinations that are hybridizable to a sample comprising nucleic acids having a target SNP, whereby such hybridization is indicative of the presence of the target SNP.

Several techniques are well-known in the art for attaching nucleic acids to a solid substrate such as a glass slide. One method is to incorporate modified bases or analogs that contain a moiety that is capable of attachment to a solid substrate, such as an amine group, a derivative of an amine group or another group with a positive charge, into nucleic acid molecules that are synthesized. The synthesized product is then contacted with a solid substrate, such as a glass slide, which is coated with an aldehyde or another reactive group which will form a covalent link with the reactive group that is on the amplified product and become covalently attached to the glass slide. Other methods, such as those using amino propyl silica surface chemistry, are also known in the art, as disclosed at world wide web at cmt.corning.com and cmgm.stanford.edu/pbrownl.

Attachment of groups to oligonucleotides which could be later converted to reactive groups is also possible using methods known in the art. Any attachment to nucleotides of oligonucleotides will become part of oligonucleotide, which could then be attached to the solid surface of the microarray. Amplified nucleic acids can be further modified, such as through cleavage into fragments or by attachment of detectable labels, prior to or following attachment to the solid substrate, as required and/or permitted by the techniques used.

For use in the applications described or suggested above, kits or articles of manufacture are also provided. Such kits can comprise a carrier means being compartmentalized to receive in close confinement one or more container means such as vials, tubes, and the like, each of the container means comprising one of the separate elements to be used in the method. For example, one of the container means can comprise a probe that is or can be detectably labeled. Such probe can be a polynucleotide specific for a polynucleotide comprising a SNP described herein. Where the kit utilizes nucleic acid hybridization to detect the target nucleic acid, the kit can also have containers containing nucleotide(s) for amplification of the target nucleic acid sequence and/or a container comprising a reporter means, such as a biotin-binding protein, such as avidin or streptavidin, bound to a reporter molecule, such as an enzymatic, fluorescent, or radioisotope label.

Kits will typically comprise the container described above and one or more other containers comprising materials desirable from a commercial and user standpoint, including buffers, diluents, filters, needles, syringes, and package inserts with instructions for use. A label may be present on the container to indicate that the composition is used for a specific therapy or non-therapeutic application, and may also indicate directions for either in vivo or in vitro use, such as those described above.

Other optional components in the kit include one or more buffers (e.g., block buffer, wash buffer, substrate buffer, etc.), other reagents such as substrate (e.g., chromogen) which is chemically altered by an enzymatic label, epitope retrieval solution, control samples (positive and/or negative controls), control slide(s) etc. An additional component is an enzyme, for example, including but not limited to, a nuclease, a ligase, or a polymerase.

SNPs are the most common type of genetic variation among people. Each SNP represents a difference in a single DNA building block, called a nucleotide. For example, a SNP may replace the nucleotide cytosine (C) with the nucleotide thymine (T) in a certain stretch of DNA. These variations may be unique or occur in many individuals. Most commonly, these variations are found in the DNA between genes.

The measurement of genetic variations of SNPs between members of a species is called SNP genotyping. It is a form of genotyping, which is the measurement of more general genetic variation.

The genetic variations of SNPs can be detected by any methods known to those skilled in the art. Such methods include, but are not limited to, DNA sequencing; primer extension assays, including allele-specific nucleotide incorporation assays and allele-specific primer extension assays (e.g., allele-specific PCR, allele-specific ligation chain reaction (LCR), and gap-LCR); allele-specific oligonucleotide hybridization assays (e.g., oligonucleotide ligation assays); cleavage protection assays in which protection from cleavage agents is used to detect mismatched bases in nucleic acid duplexes; analysis of MutS protein binding; electrophoretic analysis comparing the mobility of variant and wild type nucleic acid molecules; denaturing-gradient gel electrophoresis (DGGE, as in, e.g., Myers et al. (1985) Nature 313:495); analysis of RNase cleavage at mismatched base pairs; analysis of chemical or enzymatic cleavage of heteroduplex DNA; mass spectrometry (e.g., MALDI-TOF); genetic bit analysis (GBA); 5′ nuclease assays (e.g., TaqMan®); and assays employing molecular beacons. Certain of these methods are discussed in further detail below.

Detection of variations in target nucleic acids may be accomplished by molecular cloning and sequencing of the target nucleic acids using techniques well known in the art. Alternatively, amplification techniques such as the polymerase chain reaction (PCR) can be used to amplify target nucleic acid sequences directly from a genomic DNA preparation from tumor tissue. The nucleic acid sequence of the amplified sequences can then be determined and variations identified therefrom.

Variations can also be detected by mismatch detection methods. Mismatches are hybridized nucleic acid duplexes which are not 100% complementary. The lack of total complementarity may be due to deletions, insertions, inversions, or substitutions.

Detailed information on SNPs can be available from Single Nucleotide Polymorphism Database (dbSNP), a free public archive for genetic variation within and across different species developed and hosted by the National Center for Biotechnology Information (NCBI) in collaboration with the National Human Genome Research Institute (NHGRI). In dbSNP, a SNP is identified by a reference SNP ID number (“rs#”).

In another general aspect, provided is a combination for predicting low risk of relapse after viral suppression in the treatment of a chronic hepatitis B (CHB) infection in a subject in need thereof, the combination comprising a reagent capable of detecting the one or more of the immunogenetic biomarkers described in the application.

In certain embodiments, the detection of the immunogenetic biomarkers are performed before, during, or after the viral suppression treatment.

In certain embodiments, the combination further comprises one or more therapeutic agents for treating the CHB.

In another general aspect, provided is a kit for serological HLA typing, or a kit for genetic HLA typing, such as a DNA chip, a PCR kit, or a set of PCR primers and/or probes, for use in predicting efficacy of a viral suppression agent in treating a chronic hepatitis B (CHB) infection in a subject in need thereof.

Methods of Use

Provided herein are methods of predicting a relapse or a sustained clinical response after discontinuation of a treatment in a chronic hepatitis B (CHB) infection in a subject in need thereof using the immunogenetic biomarkers according to an embodiment of the invention. The immunogenetic biomarkers can also be used for other purposes, such as to stratify patients in clinical trials, reduce sample size in proof of concept trials by excluding subpopulations, and balance treatment arms in clinical trials.

In one general aspect, the application provides a method of determining whether a subject having a chronic hepatitis B (CHB) infection has a high or low probability of relapse after discontinuation of a viral suppression treatment, the method comprising:
a. detecting in a biological sample obtained from the subject the presence of one or more immunogenetic biomarkers of viral control of the CHB infection, wherein the one or more immunogenetic biomarkers are selected from the group consisting of:
   i. a human leukocyte antigen (HLA) allele;
   ii. one or more HLA-C single nucleotide polymorphisms (SNPs);
   iii. a HLA evolutionary diversity (HED) score; and
   iv. leucocyte receptor complex (LCR) SNPs;
b. determining that the probability of relapse is low for the subject if the one or more immunogenetic biomarkers are detected in the biological sample, or determining that the probability of relapse is high for the subject if none of the immunogenetic biomarkers is detected in the biological sample.

Preferably, the method comprises determining whether a subject having a chronic hepatitis B (CHB) infection has a high or low probability of relapse within a period of two years after the discontinuation of the viral suppression treatment.

In certain embodiments, the prediction of the probability of relapse is performed before, during, or after the viral suppression treatment of CHB.

As used herein, when the immunogenetic biomarker is a HED score, “detecting in a biological sample obtained from the subject the presence of one or more immunogenetic biomarkers of viral control of the CHB infection” refers to determining a HED score in the biological sample.

In certain embodiments, the immunogenetic biomarker comprises the presence of an HLA allele of B*51 or C*15, or the absence of an HLA allele C*07. The presence of an HLA allele of B*51 or C*15, or the absence of an HLA allele C*07 is predictive of a low probability of relapse after the discontinuation of the viral suppression treatment, preferably a low probability of a virological relapse. The absence of an HLA allele of B*51 or C*15, or the presence of an HLA allele C*07 is predictive of a high probability of relapse after the discontinuation of the viral suppression treatment, preferably a low probability of a virological relapse. In further embodiments, the HLA allele of C*15 is C*15:02 and the HLA allele C*07 is C*07:02.

In certain embodiments, the immunogenetic biomarker comprises an HLA-C SNP selected from the group consisting of rs2394952, rs3130542, rs2894202, rs9264523, rs1049281, rs9264643, rs1130838, rs2394888, AX-83089411, rs2308622, rs9264416, rs2001181, rs3132499, rs3130532, rs3130941, rs3130528, rs3134782, rs3134769, rs3130521, rs3130695, rs3130685, rs2524119, rs3130527, rs2894186, rs3130439, rs3095254, rs9264189, rs2394943, rs9394047, rs3130948, rs9368666, rs3130942, rs3130688, rs3130536, rs3134750, rs4084090, rs9264127, rs9264039, and rs3868078, or a complementary sequence thereof. The presence of the HLA-C SNP described herein is predictive of a low probability of relapse after the discontinuation of the viral suppression treatment. The absence of the HLA-C SNP described herein is predictive of a high probability of relapse after the discontinuation of the viral suppression treatment.

In further embodiments, the HLA-C SNP is selected from the group consisting of rs2394952, rs3130542, rs2894202, rs9264523, rs1049281, rs9264643, rs1130838, rs2394888, AX-83089411, rs2308622, rs9264416, rs2001181, rs3132499, rs3130532, rs3130941, rs3130528, rs3134782, rs3134769, rs3130521, rs3130695, rs3130685, rs2524119, rs3130527, rs2894186, rs3130439, rs3095254, rs9264189, rs2394943, rs9394047, rs3130948, rs9368666, rs3130942, rs3130688, rs3130536, and rs3134750, or a complementary sequence thereof. The HLC-C SNPs described herein can be used to predict the probability of a virological relapse after the discontinuation of the viral suppression treatment of CHB.

In further embodiments, the HLA-C SNP is selected from the group consisting of rs4084090, rs9264127, rs9264039, and rs3868078, or a complementary sequence thereof. The HLC-C SNPs described herein can be used to predict the probability of a clinical relapse after the discontinuation of the viral suppression treatment of CHB.

In certain embodiments, the immunogenetic biomarker comprises at least one HED score larger than the threshold value selected from the table below:

| HLA Regions | HLA gene | Variable | Threshold |
|---|---|---|---|
| Mean HLA Class I | HLA-A | HED_HLA_A_CAT_VR | 3.88 |
| Mean HLA Class I | HLA-B | HED_HLA_B_CAT_VR | 9.37 |
| Mean HLA Class I | HLA-C | HED_HLA_C_CAT_VR | 0.00 |
| Mean HLA Class I | MEAN_MHCI | HED_MEANI_CAT_VR | 8.69 |
| Mean HLA Class II | HLA-DPB1 | HED_HLA_P_CAT_VR | 2.21 |
| Mean HLA Class II | HLA-DQB1 | HED_HLA_Q_CAT_VR | 11.98 |
| Mean HLA Class II | HLA-DRB1 | HED_HLA_R_CAT_VR | 12.68 |
| Mean HLA Class II | MEAN_MHCII | HED_MEANII_CAT_VR | 7.55 |
| Mean HLA Class I | HLA-A | HED_HLA_A_CAT_CR | 3.88 |
| Mean HLA Class II | MEAN_MHCII | HED_MEANII_CAT_CR | 7.67 |
| Mean HLA Class I | HLA-C | HED_HLA_C_CAT_SCR | 7.37 |
| Mean HLA Class I | MEAN_MHCI | HED_MEANI_CAT_SCR | 7.61 |
| Mean HLA Class II | HLA-DRB1 | HED_HLA_R_CAT_SCR | 14.16 |
| Mean HLA Class II | MEAN_MHCII | HED_MEANII_CAT_SCR | 8.21 |

The presence of the HED score described herein is predictive of a low probability of relapse after the discontinuation of the viral suppression treatment. The absence of the HED score described herein is predictive of a high probability of relapse after the discontinuation of the viral suppression treatment.

In further embodiments, the HED score is selected from the group consisting of a value of HED HLA-A larger than 3.88, a value of HED HLA-B larger than 9.37, a value of HED HLA-C larger than 0.00, a value of mean HED HLA Class I larger than 8.69, a value of HED HLA-DPB1 larger than 2.21, a value of HED HLA-DQB1 larger than 11.98, a value of HED HLA-DRB1 larger than 12.68, and a value of mean HED HLA Class II larger than 7.55. The HED scores described herein can be used to predict the probability of a virological relapse after the discontinuation of the viral suppression treatment of CHB.

In further embodiments, the HED score is selected from the group consisting of a value of HED HLA-A larger than 3.88, and a value of mean HED HLA Class II larger than 7.67. The HED scores described herein can be used to predict the probability of a clinical relapse after the discontinuation of the viral suppression treatment of CHB.

In further embodiments, the HED score is selected from the group consisting of a value of HED HLA-C larger than 7.37, a value of mean HED HLA Class I larger than 7.61, a value of HED HLA-DRB1 larger than 14.16, and a value of mean HED HLA Class II larger than 8.21. The HED scores described herein can be used to predict the probability of a sustained clinical response after the discontinuation of the viral suppression treatment of CHB.

In certain embodiments, the immunogenetic biomarker comprises a LCR SNP selected from group consisting of rs10426302, rs59537494, rs28366008, rs36625, rs635608, rs7595, rs731170, rs28513, rs12459334, rs11666535, rs4806807, rs11084367, rs39714, rs1654474, rs12462907, rs775893, rs10416527, rs4441391, rs40167, rs11084339, rs775875, rs2304225, rs4077076, rs4442924, rs4806527, rs12608979, rs3765013 of COSV52557220, rs12462181, rs2075731 of COSV52550169, rs12608988, rs190480734, rs1654452, rs11879415, rs653560, rs11084387, rs11672111, rs10424969, rs77389424, rs3745902 of CM1111041, rs11672983, rs17836364, rs34549987, rs11668526, rs11667105, rs1749282, rs1654660, rs73618328, rs270785, rs76522818, rs62131745, rs2241384, rs1325158, rs10500318, rs3816051, rs34508934, rs12460627, rs11667812, rs12974194, rs17836364, rs11669431, rs12984962, rs1761462, rs4806464, rs34190750, rs28681595, rs12610675, rs12463051, rs1749282, rs1654660, rs41275824, rs2241384, rs12983338, rs272408, rs10412569, AX-3232794851, rs622941, and rs60690598, or a complementary sequence thereof. The presence of the LCR SNP described herein is predictive of a low probability of relapse after the discontinuation of the viral suppression treatment. The absence of the LCR SNP described herein is predictive of a high probability of relapse after the discontinuation of the viral suppression treatment.

In further embodiments, the LCR SNP is selected from the group consisting of rs10426302, rs59537494, rs28366008, rs36625, rs635608, rs7595, rs731170, rs28513, rs12459334, rs11666535, rs4806807, rs11084367, rs39714, rs1654474, rs12462907, rs775893, rs10416527, rs4441391, rs40167, rs11084339, rs775875, rs2304225, rs4077076, rs4442924, rs4806527, rs12608979, rs3765013 of COSV52557220, rs12462181, rs2075731 of COSV52550169, rs12608988, rs190480734, rs1654452, rs11879415, rs653560, rs11084387, rs11672111, rs10424969, rs77389424, rs3745902 of CM1111041, rs11672983, rs17836364, rs34549987, rs11668526, rs11667105, rs1749282, rs1654660, rs73618328, rs270785, rs76522818, rs62131745, rs2241384, rs1325158, rs10500318, rs3816051, rs34508934, and rs12460627, or a complementary sequence thereof. The LCR SNPs described herein can be used to predict the probability of a virological relapse after the discontinuation of the viral suppression treatment of CHB.

In further embodiments, the LCR SNP is selected from the group consisting of rs11667812, rs12974194, rs17836364, rs11669431, rs12984962, rs1761462, rs4806464, rs34190750, rs28681595, rs12610675, rs12463051, rs1749282, rs1654660, rs41275824, rs2241384, rs12983338, rs272408, rs10412569, AX-3232794851, rs622941, and rs60690598, or a complementary sequence thereof. The LCR SNPs described herein can be used to predict the probability of a clinical relapse after the discontinuation of the viral suppression treatment of CHB.

In certain embodiments, the method further comprises detecting in the biological sample the presence of one or more HLA-C alleles selected from the group consisting of allele A in rs2394952, allele G in rs3130542, allele G in rs2894202, allele A in rs9264523, allele C in rs1049281, allele A in rs9264643, allele A in rs1130838, allele G in rs2394888, allele T in AX-83089411, allele C in rs2308622, allele A in rs9264416, allele T in rs2001181, allele T in rs3132499, allele G in rs3130532, allele G in rs3130941, allele C in rs3130528, allele A in rs3134782, allele C in rs3134769, allele C in rs3130521, allele G in rs3130695, allele T in rs3130685, allele C in rs2524119, allele C in rs3130527, allele C in rs2894186, allele A in rs3130439, allele G in rs3095254, allele C in rs9264189, allele C in rs2394943, allele C in rs9394047, allele T in rs3130948, allele G in rs9368666, allele T in rs3130942, allele T in rs3130688, allele G in rs3130536, allele G in rs3134750, allele G in rs4084090, allele G in rs9264127, allele A in rs9264039, and allele T in rs3868078, or a complementary sequence thereof. The HLA-C alleles described herein are protective against relapse after the discontinuation of the viral suppression treatment of CHB.

In certain embodiments, the method further comprises detecting in the biological sample the presence of one or more HLA-C alleles selected from the group consisting of allele A in rs2394952, allele G in rs3130542, allele G in rs2894202, allele A in rs9264523, allele C in rs1049281, allele A in rs9264643, allele A in rs1130838, allele G in rs2394888, allele T in AX-83089411, allele C in rs2308622, allele A in rs9264416, allele T in rs2001181, allele T in rs3132499, allele G in rs3130532, allele G in rs3130941, allele C in rs3130528, allele A in rs3134782, allele C in rs3134769, allele C in rs3130521, allele G in rs3130695, allele T in rs3130685, allele C in rs2524119, allele C in rs3130527, allele C in rs2894186, allele A in rs3130439, allele G in rs3095254, allele C in rs9264189, allele C in rs2394943, allele C in rs9394047, allele T in rs3130948, allele G in rs9368666, allele T in rs3130942, allele T in rs3130688, allele G in rs3130536, and allele G in rs3134750, or a complementary sequence thereof. The HLA-C alleles described herein are protective against virological relapse after the discontinuation of the viral suppression treatment of CHB.

In certain embodiments, the method further comprises detecting in the biological sample the presence of one or more HLA-C alleles selected from the group consisting of allele G in rs4084090, allele G in rs9264127, allele A in rs9264039, and allele T in rs3868078, or a complementary sequence thereof. The HLA-C alleles described herein are protective against clinical relapse after the discontinuation of the viral suppression treatment of CHB.

In certain embodiments, the method further comprises detecting in the biological sample the presence of one or more LCR alleles selected from the group consisting of allele A in rs10426302, allele C in rs59537494, allele C in rs28366008, allele G in rs36625, allele T in rs635608, allele C in rs7595, allele A in rs731170, allele A in rs28513, allele T in rs12459334, allele T in rs11666535, allele T in rs4806807, allele A in rs11084367, allele G in rs39714, allele C in rs1654474, allele C in rs12462907, allele G in rs775893, allele A in rs10416527, allele A in rs4441391, allele A in rs40167, allele A in rs11084339, allele A in rs775875, allele C in rs2304225, allele A in rs4077076, allele A in rs4442924, allele A in rs4806527, allele C in rs12608979, allele A in rs3765013 of COSV52557220, allele T in rs12462181, allele T in rs2075731 of COSV52550169, allele G in rs12608988, allele G in rs190480734, allele G in rs1654452, allele T in rs11879415, allele G in rs653560, allele G in rs11084387, allele G in rs11672111, allele T in rs10424969, allele T in rs77389424, allele C in rs3745902 of CM1111041, allele G in rs11672983, allele A in rs17836364, allele C in rs34549987, allele T in rs11668526, allele G in rs11667105, allele G in rs1749282, allele A in rs1654660, allele C in rs73618328, allele C in rs270785, allele C in rs76522818, allele C in rs62131745, allele G in rs2241384, allele T in rs1325158, allele G in rs10500318, allele T in rs3816051, allele C in rs34508934, allele G in rs12460627, allele C in rs11667812, allele C in rs12974194, allele A in rs17836364, allele C in rs11669431, allele C in rs12984962, allele T in rs1761462, allele C in rs4806464, allele C in rs34190750, allele T in rs28681595, allele G in rs12610675, allele A in rs12463051, allele G in rs1749282, allele A in rs1654660, allele C in rs41275824, allele G in rs2241384, allele T in rs12983338, allele T in rs272408, allele G in rs10412569, allele T in AX-3232794851, allele G in rs622941, and allele C in rs60690598, or a complementary sequence thereof. The LCR alleles described herein are protective against relapse after the discontinuation of the viral suppression treatment of CHB.

In certain embodiments, the method further comprises detecting in the biological sample the presence of one or more LCR alleles selected from the group consisting of allele A in rs10426302, allele C in rs59537494, allele C in rs28366008, allele G in rs36625, allele T in rs635608, allele C in rs7595, allele A in rs731170, allele A in rs28513, allele T in rs12459334, allele T in rs11666535, allele T in rs4806807, allele A in rs11084367, allele G in rs39714, allele C in rs1654474, allele C in rs12462907, allele G in rs775893, allele A in rs10416527, allele A in rs4441391, allele A in rs40167, allele A in rs11084339, allele A in rs775875, allele C in rs2304225, allele A in rs4077076, allele A in rs4442924, allele A in rs4806527, allele C in rs12608979, allele A in rs3765013 of COSV52557220, allele T in rs12462181, allele T in rs2075731 of COSV52550169, allele G in rs12608988, allele G in rs190480734, allele G in rs1654452, allele T in rs11879415, allele G in rs653560, allele G in rs11084387, allele G in rs11672111, allele T in rs10424969, allele T in rs77389424, allele C in rs3745902 of CM1111041, allele G in rs11672983, allele A in rs17836364, allele C in rs34549987, allele T in rs11668526, allele G in rs11667105, allele G in rs1749282, allele A in rs1654660, allele C in rs73618328, allele C in rs270785, allele C in rs76522818, allele C in rs62131745, allele G in rs2241384, allele T in rs1325158, allele G in rs10500318, allele T in rs3816051, allele C in rs34508934, and allele G in rs12460627, or a complementary sequence thereof. The LCR alleles described herein are protective against virological relapse after the discontinuation of the viral suppression treatment of CHB.

In certain embodiments, the method further comprises detecting in the biological sample the presence of one or more LCR alleles selected from the group consisting of allele C in rs11667812, allele C in rs12974194, allele A in rs17836364, allele C in rs11669431, allele C in rs12984962, allele T in rs1761462, allele C in rs4806464, allele C in rs34190750, allele T in rs28681595, allele G in rs12610675, allele A in rs12463051, allele G in rs1749282, allele A in rs1654660, allele C in rs41275824, allele G in rs2241384, allele T in rs12983338, allele T in rs272408, allele G in rs10412569, allele T in AX-3232794851, allele G in rs622941, and allele C in rs60690598, or a complementary sequence thereof. The LCR alleles described herein are protective against clinical relapse after the discontinuation of the viral suppression treatment of CHB.

In certain embodiments, the viral suppression treatment for CHB infection is a NUC treatment. The NUC can be tenofovir, entecavir, lamivudine, adefovir, or telbivudine.

In certain embodiments, the method further comprises detecting one or more additional biomarkers associated with the relapse. Examples of such biomarkers include, but are not limited to, the level of HBsAg at the end-of-treatment, the level of HBeAg prior to treatment, HBV DNA level, ALT, AST, and HBV RNA.

Provided herein are also methods of treating a chronic hepatitis B (CHB) infection in a subject in need thereof using the immunogenetic biomarkers described herein.

In one general aspect, the application provides a method of treating a chronic hepatitis B (CHB) infection in a subject in need thereof, the method comprising:
a. administering to the subject a viral suppression treatment to treat the CHB infection;
b. detecting in a biological sample obtained from the subject the presence of one or more immunogenetic biomarkers of viral control of the CHB infection, wherein the one or more immunogenetic biomarkers are selected from the group consisting of:
   i. a human leukocyte antigen (HLA) allele;
   ii. one or more HLA-C single nucleotide polymorphisms (SNPs);
   iii. a HLA evolutionary diversity (HED) score; and
   iv. leucocyte receptor complex (LCR) SNPs;
c. if the one or more immunogenetic biomarkers of step b are detected in the biological sample, discontinuing the viral suppression treatment once viral suppression is achieved, preferably the viral suppression treatment is discontinued after 2 years of treatment; or
if none of the immunogenetic biomarkers of step b is detected in the biological sample, continuing the viral suppression treatment even after viral suppression is achieved, and/or administering to the subject a further or different viral suppression treatment.

As used herein, when the immunogenetic biomarker is a HED score, “detecting in a biological sample obtained from the subject the presence of one or more immunogenetic biomarkers of viral control of the CHB infection” refers to determining a HED score in the biological sample.

In certain embodiments, the detection of the immunogenetic biomarkers is performed after the administration of the viral suppression treatment.

In certain embodiments, provided is method of predicting a relapse after discontinuation of a viral suppression treatment in a subject diagnosed with CHB using immunogenetic biomarkers according to an embodiment of the invention. The method comprises: (a) obtaining a biological sample from a subject diagnosed with CHB; (b) determining whether the biological sample comprises one or more immunogenetic biomarkers described herein.

In certain embodiments, the immunogenetic biomarker comprises the presence of an HLA allele of B*51 or C*15, or the absence of an HLA allele C*07. The presence of an HLA allele of B*51 or C*15, or the absence of an HLA allele C*07 is predictive of a low probability of relapse after the discontinuation of the viral suppression treatment, preferably a low probability of a virological relapse. The absence of an HLA allele of B*51 or C*15, or the presence of an HLA allele C*07 is predictive of a high probability of relapse after the discontinuation of the viral suppression treatment, preferably a low probability of a virological relapse. In further embodiments, the HLA allele of C*15 is C*15:02 and the HLA allele C*07 is C*07:02.

In certain embodiments, the immunogenetic biomarker comprises an HLA-C SNP selected from the group consisting of rs2394952, rs3130542, rs2894202, rs9264523, rs1049281, rs9264643, rs1130838, rs2394888, AX-83089411, rs2308622, rs9264416, rs2001181, rs3132499, rs3130532, rs3130941, rs3130528, rs3134782, rs3134769, rs3130521, rs3130695, rs3130685, rs2524119, rs3130527, rs2894186, rs3130439, rs3095254, rs9264189, rs2394943, rs9394047, rs3130948, rs9368666, rs3130942, rs3130688, rs3130536, rs3134750, rs4084090, rs9264127, rs9264039, and rs3868078, or a complementary sequence thereof. The presence of the HLA-C SNP described herein is predictive of a low probability of relapse after the discontinuation of the viral suppression treatment. The absence of the HLA-C SNP described herein is predictive of a high probability of relapse after the discontinuation of the viral suppression treatment.

In further embodiments, the HLA-C SNP is selected from the group consisting of rs2394952, rs3130542, rs2894202, rs9264523, rs1049281, rs9264643, rs1130838, rs2394888, AX-83089411, rs2308622, rs9264416, rs2001181, rs3132499, rs3130532, rs3130941, rs3130528, rs3134782, rs3134769, rs3130521, rs3130695, rs3130685, rs2524119, rs3130527, rs2894186, rs3130439, rs3095254, rs9264189, rs2394943, rs9394047, rs3130948, rs9368666, rs3130942, rs3130688, rs3130536, and rs3134750, or a complementary sequence thereof. The HLC-C SNPs described herein can be used to predict the probability of a virological relapse after the discontinuation of the viral suppression treatment of CHB.

In further embodiments, the HLA-C SNP is selected from the group consisting of rs4084090, rs9264127, rs9264039, and rs3868078, or a complementary sequence thereof. The HLC-C SNPs described herein can be used to predict the probability of a clinical relapse after the discontinuation of the viral suppression treatment of CHB.

In certain embodiments, the immunogenetic biomarker comprises at least one HED score larger than the threshold value selected from the table below:

| HLA Regions | HLA gene | Variable | Threshold |
|---|---|---|---|
| Mean HLA Class I | HLA-A | HED_HLA_A_CAT_VR | 3.88 |
| Mean HLA Class I | HLA-B | HED_HLA_B_CAT_VR | 9.37 |
| Mean HLA Class I | HLA-C | HED_HLA_C_CAT_VR | 0.00 |
| Mean HLA Class I | MEAN_MHCI | HED_MEANI_CAT_VR | 8.69 |
| Mean HLA Class II | HLA-DPB1 | HED_HLA_P_CAT_VR | 2.21 |
| Mean HLA Class II | HLA-DQB1 | HED_HLA_Q_CAT_VR | 11.98 |
| Mean HLA Class II | HLA-DRB1 | HED_HLA_R_CAT_VR | 12.68 |
| Mean HLA Class II | MEAN_MHCII | HED_MEANII_CAT_VR | 7.55 |
| Mean HLA Class I | HLA-A | HED_HLA_A_CAT_CR | 3.88 |
| Mean HLA Class II | MEAN_MHCII | HED_MEANII_CAT_CR | 7.67 |
| Mean HLA Class I | HLA-C | HED_HLA_C_CAT_SCR | 7.37 |
| Mean HLA Class I | MEAN_MHCI | HED_MEANI_CAT_SCR | 7.61 |
| Mean HLA Class II | HLA-DRB1 | HED_HLA_R_CAT_SCR | 14.16 |
| Mean HLA Class II | MEAN_MHCII | HED_MEANII_CAT_SCR | 8.21 |

The presence of the HED score described herein is predictive of a low probability of relapse after the discontinuation of the viral suppression treatment. The absence of the HED score described herein is predictive of a high probability of relapse after the discontinuation of the viral suppression treatment.

In further embodiments, the HED score is selected from the group consisting of a value of HED HLA-A larger than 3.88, a value of HED HLA-B larger than 9.37, a value of HED HLA-C larger than 0.00, a value of mean HED HLA Class I larger than 8.69, a value of HED HLA-DPB1 larger than 2.21, a value of HED HLA-DQB1 larger than 11.98, a value of HED HLA-DRB1 larger than 12.68, and a value of mean HED HLA Class II larger than 7.55. The HED scores described herein can be used to predict the probability of a virological relapse after the discontinuation of the viral suppression treatment of CHB.

In further embodiments, the HED score is selected from the group consisting of a value of HED HLA-A larger than 3.88, and a value of mean HED HLA Class II larger than 7.67. The HED scores described herein can be used to predict the probability of a clinical relapse after the discontinuation of the viral suppression treatment of CHB.

In further embodiments, the HED score is selected from the group consisting of a value of HED HLA-C larger than 7.37, a value of mean HED HLA Class I larger than 7.61, a value of HED HLA-DRB1 larger than 14.16, and a value of mean HED HLA Class II larger than 8.21. The HED scores described herein can be used to predict the probability of a sustained clinical response after the discontinuation of the viral suppression treatment of CHB.

In certain embodiments, the immunogenetic biomarker comprises a LCR SNP selected from group consisting of rs10426302, rs59537494, rs28366008, rs36625, rs635608, rs7595, rs731170, rs28513, rs12459334, rs11666535, rs4806807, rs11084367, rs39714, rs1654474, rs12462907, rs775893, rs10416527, rs4441391, rs40167, rs11084339, rs775875, rs2304225, rs4077076, rs4442924, rs4806527, rs12608979, rs3765013 of COSV52557220, rs12462181, rs2075731 of COSV52550169, rs12608988, rs190480734, rs1654452, rs11879415, rs653560, rs11084387, rs11672111, rs10424969, rs77389424, rs3745902 of CM1111041, rs11672983, rs17836364, rs34549987, rs11668526, rs11667105, rs1749282, rs1654660, rs73618328, rs270785, rs76522818, rs62131745, rs2241384, rs1325158, rs10500318, rs3816051, rs34508934, rs12460627, rs11667812, rs12974194, rs17836364, rs11669431, rs12984962, rs1761462, rs4806464, rs34190750, rs28681595, rs12610675, rs12463051, rs1749282, rs1654660, rs41275824, rs2241384, rs12983338, rs272408, rs10412569, AX-3232794851, rs622941, and rs60690598, or a complementary sequence thereof. The presence of the LCR SNP described herein is predictive of a low probability of relapse after the discontinuation of the viral suppression treatment. The absence of the LCR SNP described herein is predictive of a high probability of relapse after the discontinuation of the viral suppression treatment.

In further embodiments, the LCR SNP is selected from the group consisting of rs10426302, rs59537494, rs28366008, rs36625, rs635608, rs7595, rs731170, rs28513, rs12459334, rs11666535, rs4806807, rs11084367, rs39714, rs1654474, rs12462907, rs775893, rs10416527, rs4441391, rs40167, rs11084339, rs775875, rs2304225, rs4077076, rs4442924, rs4806527, rs12608979, rs3765013 of COSV52557220, rs12462181, rs2075731 of COSV52550169, rs12608988, rs190480734, rs1654452, rs11879415, rs653560, rs11084387, rs11672111, rs10424969, rs77389424, rs3745902 of CM1111041, rs11672983, rs17836364, rs34549987, rs11668526, rs11667105, rs1749282, rs1654660, rs73618328, rs270785, rs76522818, rs62131745, rs2241384, rs1325158, rs10500318, rs3816051, rs34508934, and rs12460627, or a complementary sequence thereof. The LCR SNPs described herein can be used to predict the probability of a virological relapse after the discontinuation of the viral suppression treatment of CHB.

In further embodiments, the LCR SNP is selected from the group consisting of rs11667812, rs12974194, rs17836364, rs11669431, rs12984962, rs1761462, rs4806464, rs34190750, rs28681595, rs12610675, rs12463051, rs1749282, rs1654660, rs41275824, rs2241384, rs12983338, rs272408, rs10412569, AX-3232794851, rs622941, and rs60690598, or a complementary sequence thereof. The LCR SNPs described herein can be used to predict the probability of a clinical relapse after the discontinuation of the viral suppression treatment of CHB.

In certain embodiments, the method further comprises detecting in the biological sample the presence of one or more HLA-C alleles selected from the group consisting of allele A in rs2394952, allele G in rs3130542, allele G in rs2894202, allele A in rs9264523, allele C in rs1049281, allele A in rs9264643, allele A in rs1130838, allele G in rs2394888, allele T in AX-83089411, allele C in rs2308622, allele A in rs9264416, allele T in rs2001181, allele T in rs3132499, allele G in rs3130532, allele G in rs3130941, allele C in rs3130528, allele A in rs3134782, allele C in rs3134769, allele C in rs3130521, allele G in rs3130695, allele T in rs3130685, allele C in rs2524119, allele C in rs3130527, allele C in rs2894186, allele A in rs3130439, allele G in rs3095254, allele C in rs9264189, allele C in rs2394943, allele C in rs9394047, allele T in rs3130948, allele G in rs9368666, allele T in rs3130942, allele T in rs3130688, allele G in rs3130536, allele G in rs3134750, allele G in rs4084090, allele G in rs9264127, allele A in rs9264039, and allele T in rs3868078, or a complementary sequence thereof. The HLA-C alleles described herein are protective against relapse after the discontinuation of the viral suppression treatment of CHB.

In certain embodiments, the method further comprises detecting in the biological sample the presence of one or more HLA-C alleles selected from the group consisting of allele A in rs2394952, allele G in rs3130542, allele G in rs2894202, allele A in rs9264523, allele C in rs1049281, allele A in rs9264643, allele A in rs1130838, allele G in rs2394888, allele T in AX-83089411, allele C in rs2308622, allele A in rs9264416, allele T in rs2001181, allele T in rs3132499, allele G in rs3130532, allele G in rs3130941, allele C in rs3130528, allele A in rs3134782, allele C in rs3134769, allele C in rs3130521, allele G in rs3130695, allele T in rs3130685, allele C in rs2524119, allele C in rs3130527, allele C in rs2894186, allele A in rs3130439, allele G in rs3095254, allele C in rs9264189, allele C in rs2394943, allele C in rs9394047, allele T in rs3130948, allele G in rs9368666, allele T in rs3130942, allele T in rs3130688, allele G in rs3130536, and allele G in rs3134750, or a complementary sequence thereof. The HLA-C alleles described herein are protective against virological relapse after the discontinuation of the viral suppression treatment of CHB.

In certain embodiments, the method further comprises detecting in the biological sample the presence of one or more HLA-C alleles selected from the group consisting of allele G in rs4084090, allele G in rs9264127, allele A in rs9264039, and allele T in rs3868078, or a complementary sequence thereof. The HLA-C alleles described herein are protective against clinical relapse after the discontinuation of the viral suppression treatment of CHB.

In certain embodiments, the method further comprises detecting in the biological sample the presence of one or more LCR alleles selected from the group consisting of allele A in rs10426302, allele C in rs59537494, allele C in rs28366008, allele G in rs36625, allele T in rs635608, allele C in rs7595, allele A in rs731170, allele A in rs28513, allele T in rs12459334, allele T in rs11666535, allele T in rs4806807, allele A in rs11084367, allele G in rs39714, allele C in rs1654474, allele C in rs12462907, allele G in rs775893, allele A in rs10416527, allele A in rs4441391, allele A in rs40167, allele A in rs11084339, allele A in rs775875, allele C in rs2304225, allele A in rs4077076, allele A in rs4442924, allele A in rs4806527, allele C in rs12608979, allele A in rs3765013 of COSV52557220, allele T in rs12462181, allele T in rs2075731 of COSV52550169, allele G in rs12608988, allele G in rs190480734, allele G in rs1654452, allele T in rs11879415, allele G in rs653560, allele G in rs11084387, allele G in rs11672111, allele T in rs10424969, allele T in rs77389424, allele C in rs3745902 of CM1111041, allele G in rs11672983, allele A in rs17836364, allele C in rs34549987, allele T in rs11668526, allele G in rs11667105, allele G in rs1749282, allele A in rs1654660, allele C in rs73618328, allele C in rs270785, allele C in rs76522818, allele C in rs62131745, allele Gin rs2241384, allele T in rs1325158, allele G in rs10500318, allele T in rs3816051, allele C in rs34508934, allele G in rs12460627, allele C in rs11667812, allele C in rs12974194, allele A in rs17836364, allele C in rs11669431, allele C in rs12984962, allele T in rs1761462, allele C in rs4806464, allele C in rs34190750, allele T in rs28681595, allele G in rs12610675, allele A in rs12463051, allele G in rs1749282, allele A in rs1654660, allele C in rs41275824, allele G in rs2241384, allele T in rs12983338, allele T in rs272408, allele G in rs10412569, allele T in AX-3232794851, allele G in rs622941, and allele C in rs60690598, or a complementary sequence thereof. The LCR alleles described herein are protective against relapse after the discontinuation of the viral suppression treatment of CHB.

In certain embodiments, the method further comprises detecting in the biological sample the presence of one or more LCR alleles selected from the group consisting of allele A in rs10426302, allele C in rs59537494, allele C in rs28366008, allele G in rs36625, allele T in rs635608, allele C in rs7595, allele A in rs731170, allele A in rs28513, allele T in rs12459334, allele T in rs11666535, allele T in rs4806807, allele A in rs11084367, allele G in rs39714, allele C in rs1654474, allele C in rs12462907, allele G in rs775893, allele A in rs10416527, allele A in rs4441391, allele A in rs40167, allele A in rs11084339, allele A in rs775875, allele C in rs2304225, allele A in rs4077076, allele A in rs4442924, allele A in rs4806527, allele C in rs12608979, allele A in rs3765013 of COSV52557220, allele T in rs12462181, allele T in rs2075731 of COSV52550169, allele G in rs12608988, allele G in rs190480734, allele G in rs1654452, allele T in rs11879415, allele G in rs653560, allele G in rs11084387, allele G in rs11672111, allele T in rs10424969, allele T in rs77389424, allele C in rs3745902 of CM1111041, allele G in rs11672983, allele A in rs17836364, allele C in rs34549987, allele T in rs11668526, allele G in rs11667105, allele G in rs1749282, allele A in rs1654660, allele C in rs73618328, allele C in rs270785, allele C in rs76522818, allele C in rs62131745, allele G in rs2241384, allele T in rs1325158, allele G in rs10500318, allele T in rs3816051, allele C in rs34508934, and allele G in rs12460627, or a complementary sequence thereof. The LCR alleles described herein are protective against virological relapse after the discontinuation of the viral suppression treatment of CHB.

In certain embodiments, the method further comprises detecting in the biological sample the presence of one or more LCR alleles selected from the group consisting of allele C in rs11667812, allele C in rs12974194, allele A in rs17836364, allele C in rs11669431, allele C in rs12984962, allele T in rs1761462, allele C in rs4806464, allele C in rs34190750, allele T in rs28681595, allele G in rs12610675, allele A in rs12463051, allele G in rs1749282, allele A in rs1654660, allele C in rs41275824, allele G in rs2241384, allele T in rs12983338, allele T in rs272408, allele G in rs10412569, allele T in AX-3232794851, allele G in rs622941, and allele C in rs60690598, or a complementary sequence thereof. The LCR alleles described herein are protective against clinical relapse after the discontinuation of the viral suppression treatment of CHB.

In certain embodiments, the viral suppression treatment for the CHB infection is a NUC treatment. The NUC can be tenofovir, entecavir, lamivudine, adefovir, or telbivudine.

In certain embodiments, the method further comprises detecting one or more additional biomarkers associated with the relapse. Examples of such biomarkers include, but are not limited to, the level of HBsAg at the end-of-treatment, the level of HBeAg prior to treatment, HBV DNA level, ALT, AST, and HBV RNA.

In certain embodiments, the presence or absence of the one or more immunogenetic biomarkers in the biological sample is determined using any method known to one skilled in the art.

In certain embodiments, the viral suppression treatment is discontinued once viral suppression is achieved, preferably after 2 years of treatment, if the one or more the immunogenetic biomarkers are detected in the biological sample.

In certain embodiments, the viral suppression treatment is continued once viral suppression is achieved, preferably after 2 years of treatment, and/or a further or different viral suppression treatment is administered to the subject, if none of the immunogenetic biomarkers is detected in the biological sample.

In certain embodiments, the subject achieves HBV DNA <60 IU/mL, ALT <80 U/L, or HBeAg negative at or after one year, two years, three years, or four years, or anytime in between, after the viral suppression treatment. In further embodiments, the subject then discontinues the viral suppression treatment.

In further embodiments, the subject has no virological relapse or clinical relapse at or after 3 months, 6 months, 12 months, 18 months, 24 months, or 36 months after the discontinuation of the viral suppression treatment, or anytime in between, and wherein the virological relapse is identified as HBV DNA ≥2000 IU/ml or HBeAg positive, and the clinical relapse is identified as i) HBV DNA ≥2000 IU/ml or HBeAg positive, and ii) ALT ≥80 U/L.

Provided herein are also methods of treating a chronic hepatitis B (CHB) infection in a subject in need thereof, the method comprising:
a. administering to the subject a viral suppression treatment to treat the CHB infection;
b. detecting in a biological sample obtained from the subject the presence of one or more immunogenetic biomarkers of viral control of the CHB infection, wherein the one or more immunogenetic biomarkers are selected from the group consisting of:
   i. a human leukocyte antigen (HLA) allele;
   ii. one or more HLA-C single nucleotide polymorphisms (SNPs);
   iii. a HLA evolutionary diversity (HED) score; and
   iv. leucocyte receptor complex (LCR) SNPs;
c. discontinuing the viral suppression treatment when the CHB infection is suppressed in the subject, and
d. administering to the subject the viral suppression treatment or another viral suppression treatment less than two years after the discontinuation if none of the immunogenetic biomarkers is detected in the biological sample.

As used herein, when the immunogenetic biomarker is a HED score, “detecting in a biological sample obtained from the subject the presence of one or more immunogenetic biomarkers of viral control of the CHB infection” refers to determining a HED score in the biological sample.

In certain embodiments, the immunogenetic biomarker comprises the presence of an HLA allele of B*51 or C*15, or the absence of an HLA allele C*07. The presence of an HLA allele of B*51 or C*15, or the absence of an HLA allele C*07 is predictive of a low probability of relapse after the discontinuation of the viral suppression treatment, preferably a low probability of a virological relapse. The absence of an HLA allele of B*51 or C*15, or the presence of an HLA allele C*07 is predictive of a high probability of relapse after the discontinuation of the viral suppression treatment, preferably a low probability of a virological relapse. In further embodiments, the HLA allele of C*15 is C*15:02 and the HLA allele C*07 is C*07:02.

In certain embodiments, the immunogenetic biomarker comprises an HLA-C SNP selected from the group consisting of rs2394952, rs3130542, rs2894202, rs9264523, rs1049281, rs9264643, rs1130838, rs2394888, AX-83089411, rs2308622, rs9264416, rs2001181, rs3132499, rs3130532, rs3130941, rs3130528, rs3134782, rs3134769, rs3130521, rs3130695, rs3130685, rs2524119, rs3130527, rs2894186, rs3130439, rs3095254, rs9264189, rs2394943, rs9394047, rs3130948, rs9368666, rs3130942, rs3130688, rs3130536, rs3134750, rs4084090, rs9264127, rs9264039, and rs3868078, or a complementary sequence thereof. The presence of the HLA-C SNP described herein is predictive of a low probability of relapse after the discontinuation of the viral suppression treatment. The absence of the HLA-C SNP described herein is predictive of a high probability of relapse after the discontinuation of the viral suppression treatment.

In further embodiments, the HLA-C SNP is selected from the group consisting of rs2394952, rs3130542, rs2894202, rs9264523, rs1049281, rs9264643, rs1130838, rs2394888, AX-83089411, rs2308622, rs9264416, rs2001181, rs3132499, rs3130532, rs3130941, rs3130528, rs3134782, rs3134769, rs3130521, rs3130695, rs3130685, rs2524119, rs3130527, rs2894186, rs3130439, rs3095254, rs9264189, rs2394943, rs9394047, rs3130948, rs9368666, rs3130942, rs3130688, rs3130536, and rs3134750, or a complementary sequence thereof. The HLC-C SNPs described herein can be used to predict the probability of a virological relapse after the discontinuation of the viral suppression treatment of CHB.

In further embodiments, the HLA-C SNP is selected from the group consisting of rs4084090, rs9264127, rs9264039, and rs3868078, or a complementary sequence thereof. The HLC-C SNPs described herein can be used to predict the probability of a clinical relapse after the discontinuation of the viral suppression treatment of CHB.

In certain embodiments, the immunogenetic biomarker comprises at least one HED score larger than the threshold value selected from the table below:

| HLA Regions | HLA gene | Variable | Threshold |
|---|---|---|---|
| Mean HLA Class I | HLA-A | HED_HLA_A_CAT_VR | 3.88 |
| Mean HLA Class I | HLA-B | HED_HLA_B_CAT_VR | 9.37 |
| Mean HLA Class I | HLA-C | HED_HLA_C_CAT_VR | 0.00 |
| Mean HLA Class I | MEAN_MHCI | HED_MEANI_CAT_VR | 8.69 |
| Mean HLA Class II | HLA-DPB1 | HED_HLA_P_CAT_VR | 2.21 |
| Mean HLA Class II | HLA-DQB1 | HED_HLA_Q_CAT_VR | 11.98 |
| Mean HLA Class II | HLA-DRB1 | HED_HLA_R_CAT_VR | 12.68 |
| Mean HLA Class II | MEAN_MHCII | HED_MEANII_CAT_VR | 7.55 |
| Mean HLA Class I | HLA-A | HED_HLA_A_CAT_CR | 3.88 |
| Mean HLA Class II | MEAN_MHCII | HED_MEANII_CAT_CR | 7.67 |
| Mean HLA Class I | HLA-C | HED_HLA_C_CAT_SCR | 7.37 |
| Mean HLA Class I | MEAN_MHCI | HED_MEANI_CAT_SCR | 7.61 |
| Mean HLA Class II | HLA-DRB1 | HED_HLA_R_CAT_SCR | 14.16 |
| Mean HLA Class II | MEAN_MHCII | HED_MEANII_CAT_SCR | 8.21 |

The presence of the HED score described herein is predictive of a low probability of relapse after the discontinuation of the viral suppression treatment. The absence of the HED score described herein is predictive of a high probability of relapse after the discontinuation of the viral suppression treatment.

In further embodiments, the HED score is selected from the group consisting of a value of HED HLA-A larger than 3.88, a value of HED HLA-B larger than 9.37, a value of HED HLA-C larger than 0.00, a value of mean HED HLA Class I larger than 8.69, a value of HED HLA-DPB1 larger than 2.21, a value of HED HLA-DQB1 larger than 11.98, a value of HED HLA-DRB1 larger than 12.68, and a value of mean HED HLA Class II larger than 7.55. The HED scores described herein can be used to predict the probability of a virological relapse after the discontinuation of the viral suppression treatment of CHB.

In further embodiments, the HED score is selected from the group consisting of a value of HED HLA-A larger than 3.88, and a value of mean HED HLA Class II larger than 7.67. The HED scores described herein can be used to predict the probability of a clinical relapse after the discontinuation of the viral suppression treatment of CHB.

In further embodiments, the HED score is selected from the group consisting of a value of HED HLA-C larger than 7.37, a value of mean HED HLA Class I larger than 7.61, a value of HED HLA-DRB1 larger than 14.16, and a value of mean HED HLA Class II larger than 8.21. The HED scores described herein can be used to predict the probability of a sustained clinical response after the discontinuation of the viral suppression treatment of CHB.

In certain embodiments, the immunogenetic biomarker comprises a LCR SNP selected from group consisting of rs10426302, rs59537494, rs28366008, rs36625, rs635608, rs7595, rs731170, rs28513, rs12459334, rs11666535, rs4806807, rs11084367, rs39714, rs1654474, rs12462907, rs775893, rs10416527, rs4441391, rs40167, rs11084339, rs775875, rs2304225, rs4077076, rs4442924, rs4806527, rs12608979, rs3765013 of COSV52557220, rs12462181, rs2075731 of COSV52550169, rs12608988, rs190480734, rs1654452, rs11879415, rs653560, rs11084387, rs11672111, rs10424969, rs77389424, rs3745902 of CM1111041, rs11672983, rs17836364, rs34549987, rs11668526, rs11667105, rs1749282, rs1654660, rs73618328, rs270785, rs76522818, rs62131745, rs2241384, rs1325158, rs10500318, rs3816051, rs34508934, rs12460627, rs11667812, rs12974194, rs17836364, rs11669431, rs12984962, rs1761462, rs4806464, rs34190750, rs28681595, rs12610675, rs12463051, rs1749282, rs1654660, rs41275824, rs2241384, rs12983338, rs272408, rs10412569, AX-3232794851, rs622941, and rs60690598, or a complementary sequence thereof. The presence of the LCR SNP described herein is predictive of a low probability of relapse after the discontinuation of the viral suppression treatment. The absence of the LCR SNP described herein is predictive of a high probability of relapse after the discontinuation of the viral suppression treatment.

In further embodiments, the LCR SNP is selected from the group consisting of rs10426302, rs59537494, rs28366008, rs36625, rs635608, rs7595, rs731170, rs28513, rs12459334, rs11666535, rs4806807, rs11084367, rs39714, rs1654474, rs12462907, rs775893, rs10416527, rs4441391, rs40167, rs11084339, rs775875, rs2304225, rs4077076, rs4442924, rs4806527, rs12608979, rs3765013 of COSV52557220, rs12462181, rs2075731 of COSV52550169, rs12608988, rs190480734, rs1654452, rs11879415, rs653560, rs11084387, rs11672111, rs10424969, rs77389424, rs3745902 of CM1111041, rs11672983, rs17836364, rs34549987, rs11668526, rs11667105, rs1749282, rs1654660, rs73618328, rs270785, rs76522818, rs62131745, rs2241384, rs1325158, rs10500318, rs3816051, rs34508934, and rs12460627, or a complementary sequence thereof. The LCR SNPs described herein can be used to predict the probability of a virological relapse after the discontinuation of the viral suppression treatment of CHB.

In further embodiments, the LCR SNP is selected from the group consisting of rs11667812, rs12974194, rs17836364, rs11669431, rs12984962, rs1761462, rs4806464, rs34190750, rs28681595, rs12610675, rs12463051, rs1749282, rs1654660, rs41275824, rs2241384, rs12983338, rs272408, rs10412569, AX-3232794851, rs622941, and rs60690598, or a complementary sequence thereof. The LCR SNPs described herein can be used to predict the probability of a clinical relapse after the discontinuation of the viral suppression treatment of CHB.

In certain embodiments, the method further comprises detecting in the biological sample the presence of one or more HLA-C alleles selected from the group consisting of allele A in rs2394952, allele G in rs3130542, allele G in rs2894202, allele A in rs9264523, allele C in rs1049281, allele A in rs9264643, allele A in rs1130838, allele G in rs2394888, allele T in AX-83089411, allele C in rs2308622, allele A in rs9264416, allele T in rs2001181, allele T in rs3132499, allele G in rs3130532, allele G in rs3130941, allele C in rs3130528, allele A in rs3134782, allele C in rs3134769, allele C in rs3130521, allele G in rs3130695, allele T in rs3130685, allele C in rs2524119, allele C in rs3130527, allele C in rs2894186, allele A in rs3130439, allele G in rs3095254, allele C in rs9264189, allele C in rs2394943, allele C in rs9394047, allele T in rs3130948, allele G in rs9368666, allele T in rs3130942, allele T in rs3130688, allele G in rs3130536, allele G in rs3134750, allele G in rs4084090, allele G in rs9264127, allele A in rs9264039, and allele T in rs3868078, or a complementary sequence thereof. The HLA-C alleles described herein are protective against relapse after the discontinuation of the viral suppression treatment of CHB.

In certain embodiments, the method further comprises detecting in the biological sample the presence of one or more HLA-C alleles selected from the group consisting of allele A in rs2394952, allele G in rs3130542, allele G in rs2894202, allele A in rs9264523, allele C in rs1049281, allele A in rs9264643, allele A in rs1130838, allele G in rs2394888, allele T in AX-83089411, allele C in rs2308622, allele A in rs9264416, allele T in rs2001181, allele T in rs3132499, allele G in rs3130532, allele G in rs3130941, allele C in rs3130528, allele A in rs3134782, allele C in rs3134769, allele C in rs3130521, allele G in rs3130695, allele T in rs3130685, allele C in rs2524119, allele C in rs3130527, allele C in rs2894186, allele A in rs3130439, allele G in rs3095254, allele C in rs9264189, allele C in rs2394943, allele C in rs9394047, allele T in rs3130948, allele G in rs9368666, allele T in rs3130942, allele T in rs3130688, allele G in rs3130536, and allele G in rs3134750, or a complementary sequence thereof. The HLA-C alleles described herein are protective against virological relapse after the discontinuation of the viral suppression treatment of CHB.

In certain embodiments, the method further comprises detecting in the biological sample the presence of one or more HLA-C alleles selected from the group consisting of allele G in rs4084090, allele G in rs9264127, allele A in rs9264039, and allele T in rs3868078, or a complementary sequence thereof. The HLA-C alleles described herein are protective against clinical relapse after the discontinuation of the viral suppression treatment of CHB.

In certain embodiments, the method further comprises detecting in the biological sample the presence of one or more LCR alleles selected from the group consisting of allele A in rs10426302, allele C in rs59537494, allele C in rs28366008, allele G in rs36625, allele T in rs635608, allele C in rs7595, allele A in rs731170, allele A in rs28513, allele T in rs12459334, allele T in rs11666535, allele T in rs4806807, allele A in rs11084367, allele G in rs39714, allele C in rs1654474, allele C in rs12462907, allele G in rs775893, allele A in rs10416527, allele A in rs4441391, allele A in rs40167, allele A in rs11084339, allele A in rs775875, allele C in rs2304225, allele A in rs4077076, allele A in rs4442924, allele A in rs4806527, allele C in rs12608979, allele A in rs3765013 of COSV52557220, allele T in rs12462181, allele T in rs2075731 of COSV52550169, allele G in rs12608988, allele G in rs190480734, allele G in rs1654452, allele T in rs11879415, allele G in rs653560, allele G in rs11084387, allele G in rs11672111, allele T in rs10424969, allele T in rs77389424, allele C in rs3745902 of CM1111041, allele G in rs11672983, allele A in rs17836364, allele C in rs34549987, allele T in rs11668526, allele G in rs11667105, allele G in rs1749282, allele A in rs1654660, allele C in rs73618328, allele C in rs270785, allele C in rs76522818, allele C in rs62131745, allele G in rs2241384, allele T in rs1325158, allele G in rs10500318, allele T in rs3816051, allele C in rs34508934, allele G in rs12460627, allele C in rs11667812, allele C in rs12974194, allele A in rs17836364, allele C in rs11669431, allele C in rs12984962, allele T in rs1761462, allele C in rs4806464, allele C in rs34190750, allele T in rs28681595, allele G in rs12610675, allele A in rs12463051, allele G in rs1749282, allele A in rs1654660, allele C in rs41275824, allele G in rs2241384, allele T in rs12983338, allele T in rs272408, allele G in rs10412569, allele T in AX-3232794851, allele G in rs622941, and allele C in rs60690598, or a complementary sequence thereof. The LCR alleles described herein are protective against relapse after the discontinuation of the viral suppression treatment of CHB.

In certain embodiments, the method further comprises detecting in the biological sample the presence of one or more LCR alleles selected from the group consisting of allele A in rs10426302, allele C in rs59537494, allele C in rs28366008, allele G in rs36625, allele T in rs635608, allele C in rs7595, allele A in rs731170, allele A in rs28513, allele T in rs12459334, allele T in rs11666535, allele T in rs4806807, allele A in rs11084367, allele G in rs39714, allele C in rs1654474, allele C in rs12462907, allele G in rs775893, allele A in rs10416527, allele A in rs4441391, allele A in rs40167, allele A in rs11084339, allele A in rs775875, allele C in rs2304225, allele A in rs4077076, allele A in rs4442924, allele A in rs4806527, allele C in rs12608979, allele A in rs3765013 of COSV52557220, allele T in rs12462181, allele T in rs2075731 of COSV52550169, allele G in rs12608988, allele G in rs190480734, allele G in rs1654452, allele T in rs11879415, allele G in rs653560, allele G in rs11084387, allele G in rs11672111, allele T in rs10424969, allele T in rs77389424, allele C in rs3745902 of CM1111041, allele G in rs11672983, allele A in rs17836364, allele C in rs34549987, allele T in rs11668526, allele G in rs11667105, allele G in rs1749282, allele A in rs1654660, allele C in rs73618328, allele C in rs270785, allele C in rs76522818, allele C in rs62131745, allele G in rs2241384, allele T in rs1325158, allele G in rs10500318, allele T in rs3816051, allele C in rs34508934, and allele G in rs12460627, or a complementary sequence thereof. The LCR alleles described herein are protective against virological relapse after the discontinuation of the viral suppression treatment of CHB.

In certain embodiments, the method further comprises detecting in the biological sample the presence of one or more LCR alleles selected from the group consisting of allele C in rs11667812, allele C in rs12974194, allele A in rs17836364, allele C in rs11669431, allele C in rs12984962, allele T in rs1761462, allele C in rs4806464, allele C in rs34190750, allele T in rs28681595, allele G in rs12610675, allele A in rs12463051, allele G in rs1749282, allele A in rs1654660, allele C in rs41275824, allele G in rs2241384, allele T in rs12983338, allele T in rs272408, allele G in rs10412569, allele T in AX-3232794851, allele G in rs622941, and allele C in rs60690598, or a complementary sequence thereof. The LCR alleles described herein are protective against clinical relapse after the discontinuation of the viral suppression treatment of CHB.

In certain embodiments, the viral suppression treatment for the CHB infection is a NUC treatment. The NUC can be tenofovir, entecavir, lamivudine, adefovir, or telbivudine.

In certain embodiments, the viral suppression treatment for the CHB infection is a NUC treatment. The NUC can be tenofovir, entecavir, lamivudine, adefovir, or telbivudine.

In certain embodiments, the presence or absence of the one or more immunogenetic biomarkers in the biological sample is determined using any method known to one skilled in the art.

In certain embodiments, the subject achieves HBV DNA <60 IU/mL, ALT <80 U/L, or HBeAg negative at or after one year, two years, three years, or four years, or anytime in between, after the viral suppression treatment. In further embodiments, the subject then discontinues the viral suppression treatment.

In further embodiments, the subject has no virological relapse or clinical relapse at or after 3 months, 6 months, 12 months, 18 months, 24 months, or 36 months after the discontinuation of the viral suppression treatment, or anytime in between, and wherein the virological relapse is identified as HBV DNA ≥2000 IU/ml or HBeAg positive, and the clinical relapse is identified as i) HBV DNA ≥2000 IU/ml or HBeAg positive, and ii) ALT ≥80 U/L.

In certain embodiments, the sample is a tissue sample, a cellular sample, or a blood sample. Preferably, the sample is a blood sample.

In certain embodiments, the sample is obtained before, during, or after the viral suppression treatment.

In certain embodiments, the CHB infection is suppressed at the time of discontinuation of the viral suppression treatment. In one embodiment, the subject discontinues a viral suppression treatment when the subject achieves HBV DNA <60 IU/mL, ALT <80 U/L, or HBeAg negative at or after one year, two years, three years, or four years, or anytime in between, after the viral suppression treatment. Preferably, the subject achieves HBsAg <100 IU/mL at the discontinuation of the viral suppression treatment.

In certain embodiment, the method further comprises measuring HBV DNA, ALT, and HBsAg, at or after 3 months, 6 months, 12 months, 18 months, 24 months, or 36 months after the discontinuation of the viral suppression treatment, or anytime in between.

In certain embodiment, the subject has no virological relapse at or after 3 months, 6 months, 12 months, 18 months, 24 months, or 36 months after the discontinuation of the viral suppression treatment, or anytime in between, and the virological relapse is identified as HBV DNA ≥2000 IU/ml or HBeAg positive.

In certain embodiment, the subject has no clinical relapse at or after 3 months, 6 months, 12 months, 18 months, 24 months, or 36 months after the discontinuation of the viral suppression treatment, or anytime in between, and the clinical relapse is identified as i) HBV DNA ≥2000 IU/ml or HBeAg positive, and ii) ALT ≥80 U/L.

Any method known in the art or described herein can be used to detect the presence of a SNP in view of the present disclosure. According to embodiments of the application, the SNP or allele is determined by a method selected from the group consisting of DNA sequencing, restriction fragment length polymorphism (RFLP analysis), allele specific oligonucleotide (ASO) analysis, Denaturing/Temperature Gradient Gel Electrophoresis (DGGE/TGGE), Single-Strand Conformation Polymorphism (SSCP) analysis, Dideoxy fingerprinting (ddF), pyrosequencing analysis, acycloprime analysis, Reverse dot blot, GeneChip microarrays, Dynamic allele-specific hybridization (DASH), Peptide nucleic acid (PNA) and locked nucleic acids (LNA) probes, TaqMan, Molecular Beacons, Intercalating dye, FRET primers, AlphaScreen, SNPstream, genetic bit analysis (GBA), Multiplex minisequencing, SNaPshot, MassEXTEND, MassArray, GOOD assay, Microarray miniseq, arrayed primer extension (APEX), Microarray primer extension, Tag arrays, Coded microspheres, Template-directed incorporation (TDI), fluorescence polarization, Colorimetric oligonucleotide ligation assay (OLA), Sequence-coded OLA, Microarray ligation, Ligase chain reaction, Padlock probes, Rolling circle amplification, and Invader assay.

### EXAMPLES

### Example 1: Identification and Evaluation of Virological and Host Human Leukocyte Antigen (HLA) Typing Signature Associated with Onset of Virological and Clinical Relapse and Sustained Clinical Response

The objective of the current study was to evaluate virological and host human leukocyte antigen (HLA) typing signature that can be associated with relapse and sustained response in chronic hepatitis B chronic hepatitis B (CHB) patients following discontinuation of direct antiviral treatment.

### Materials and Methods

Patients and Study Design: In this multi-center prospective study in Taiwan, 242 CHB patients were enrolled while in their last half year of a minimum 3-year treatment regimen with direct antivirals. Of them, 47 were excluded due to protocol violation, screening failure, lost to follow-up, withdrawal by the subject, use of immune related therapy, or on-treatment clinical relapse. An additional 14 were excluded as the analysis required patients to be hepatitis B e-antigen (HBeAg) negative, hepatitis virus (HBV) DNA <60 IU/mL, and alanine transaminase (ALT) <80 U/L at the last on-treatment visit, leaving 186 patients eligible for analysis.

Patients were followed for up to two years after treatment discontinuation while assessing virological and clinical relapse, provided that treatment was not re-initiated. Virological relapse was defined as HBV DNA ≥2000 IU/mL. Clinical relapse was defined as ALT level ≥2× ULN in addition to a virological relapse. A patient was denoted a sustained clinical responder in case no clinical and no virological relapse occurred during the entire follow-up period after treatment cessation.

Serology: During the on-treatment period, blood samples were collected at 3 time points, with the last one collected around the last day of treatment. In the follow-up period after treatment discontinuation additional blood samples were collected at 3 or 6 months intervals, provided that treatment was not re-initiated.

HLA Typing: Each subject was HLA typed by Sanger sequencing for HLA class I region (A, B and C genes) and for HLA class II region (DRB1 and DQB1 genes). DPB1 genes in the class II region were characterized by high resolution polymerase chain reaction-sequence-based-typing (PCR-SBT) methodology1 and complemented for 21 samples showing ambiguities, by deep sequencing (TBG HLA DPB1 Typing Kit, which contains the DPB1 locus specific primers). Amplicons were sequenced using Illumina Miseq v2 300 and analyzed by Omixon Twin Software for HLA genotype analysis.

HLA Alleles: For each gene, both in class I and class II regions, the presence of a specific allele was derived applying both a dominant model (present vs. absent) and an additive model (0, 1 or 2 copies of a specific allele considered as continuous variable).

HLA Diversity: For each subject HLA-typed, for HLA class I region and HLA class II region independently, HLA genetic homozygosity has been derived per locus and per allele, comparing the proportion of homozygous with heterozygous alleles for each HLA gene (comparing for each gene or locus zero and two copies of a specific allele vs. one copy) (Chowell, et al., Science, 2018, 359: 582-7; Thursz, et. al., Nature Genetics 1997, 17: 11-2).

HLA Evolutionary Diversity (HED) score, a more complex measure taking into account the predicted influence on amino-acid composition, has been derived for each patient as a mean HED across all HLA class I and class II genes separately (Arora, et al., Molecular Biology and Evolution, 2020, 37: 639-50; Chowell, et al., Nature Medicine, 2019, 25: 1715-20) and per gene. In particular, the HLA allele protein sequence alignments were obtained from ftp://ftp.ebi.ac.uk/pub/databases/ipd/imgt/hla/alignments/ and were based on the protein sequences contained in the IMGT/HLA database (Release 3.40.0, 2020-04-20) 6. The following HLA protein loci were used for the analysis, for HLA-type I: HLA-A, HLA-B and HLA-C; HLA-type II: HLA-DPB1, HLA-DRB1 and HLA-DQB1. For each HLA protein locus, amino-acid co-ordinates of the MEW antigen-recognition domains were identified using the Ensemb1 database protein annotations. Globally the co-ordinates cover exons 2 and 3 in HLA-type I genes and exon 2 and a portion of exon 1 in HLA-type II genes.

For each subject and HLA locus, the HLA genotypes using a 4-field HLA allelic identification code were extracted. The 4-field HLA genetic codes were then used to match the alleles to the corresponding proteins in the alignment files. For each pair of alleles in the genotypes, their functional distance in terms of proteins encoded was measured using the Grantham distance metric. The Grantham distance is a quantitative pairwise distance between two amino-acids. The distance for a pair takes into account their physiochemical properties (biochemical composition, polarity and volume of each amino-acid). The Grantham overall divergence for a given alignment of a pair of sequences is calculated by summing the individual amino-acid pair distance value and then by dividing the sum by the alignment length. For the purpose of this analysis, only the MEW binding domain was considered to calculate the differences. For each patient, mean HED was calculated as the mean of divergences obtained for HLA-A, HLA-B and HLA-C for HLA-type I and HLA-DPB1, HLA-DRB1 and HLA-DQB1 for HLA-type II loci.

In addition to the derived continuous values, HED scores were also categorized into binary variables with low and high levels for further analysis. In the analysis on virological and clinical relapse, categorization thresholds were derived using the survminer R package (surv_cutpoint function) determining the optimal cut point in relation to relapse. To assess sustained clinical response logistic regression is applied, and thresholds selected corresponding to the value providing the maximum AUC (area under the curve) of the ROC (receiver operating characteristic) curve of the model including in addition to end of treatment HBsAg the categorical HED score variable.

Statistical Analysis: Continuous variables are presented as means±standard deviations, categorical variables by counts and percentages (See Table 1 below).

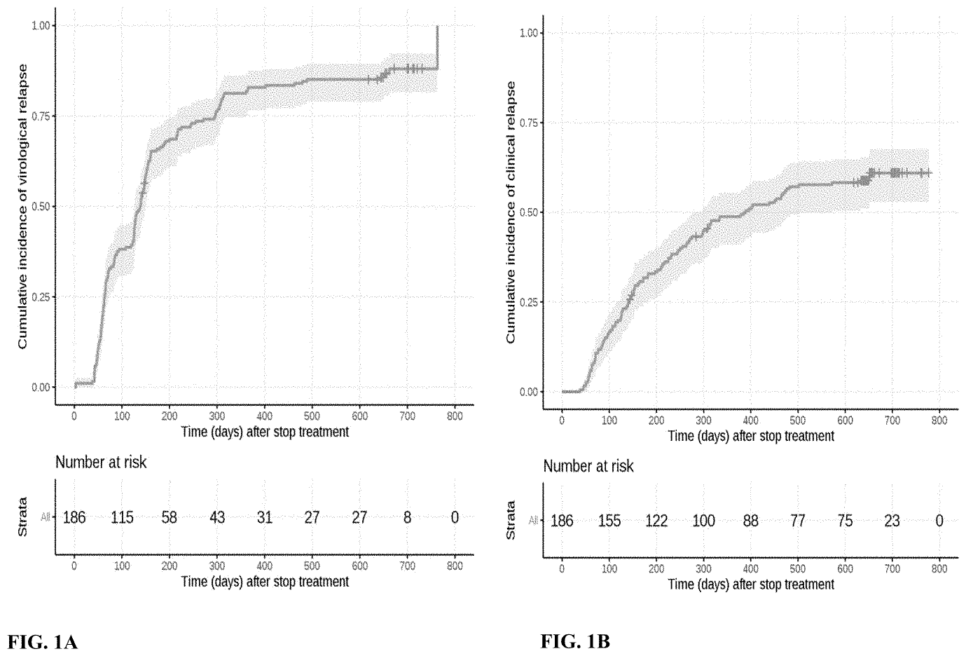

Cumulative incidence of virological and clinical relapse after stop of treatment was assessed by Kaplan-Meier curves (FIGS. 1A and 1B). In addition, Cox proportional hazard regression analysis (Yang, et al., Biometrika, 2005, 92: 937-50) was applied, obtaining univariate and multivariate models for time to virological and clinical relapse (model fits are provided in Table 2a and Table 2b for clinical and virological relapse respectively; see Table 2a and 2b below).

Statistical Analysis of HLA Data: The association between the HLA alleles (at two and four digits resolution for each locus) and diversity (genetic homozygosity and HED score, independently) and onset of relapse was assessed using a Cox model (Harrell et al., JAMA: The Journal of the American Medical Association, 1982, 247: 2543) including HBsAg level at the last visit on treatment (high (≥100 IU/mL) vs. low (<100 IU/mL)) and the antiviral treatment at enrolment (tenofovir vs. entecavir) as main effects. The association between the HLA alleles (at two and four digits resolution for each locus) and diversity (genetic homozygosity and HED score, independently) and sustained clinical response (SCR) was assessed using a logistic model including HBsAg level at the last visit on treatment (high ((≥100 IU/mL) vs. low (<100 IU/mL)) as main effect. Only HLA alleles with an observed minor allele frequency above 5% were considered.

HLA alleles and HED scores showing a false discovery rate (FDR) below 10% were considered as significant. FDR was computed per analysis (clinical relapse, virological relapse, and sustained clinical response) and across all models (additive, dominant, and homozygous).

HLA markers significantly associated with onset of relapse were explored further, in order to develop a genetic signature (based on HLA data) predictive (protective) for early onset of relapse.

Both for clinical and virological relapse, different Cox proportional hazard regression models were compared by AIC, BIC10, and concordance (Harrell's C-index) to assess the association to the hazard of relapse. In addition, for the most relevant models, time-dependent receiver operating characteristic (ROC) curves (and the corresponding area under the curve (AUC)) were compared.

For sustained clinical response (SCR), different logistic regression models were compared by AIC and BIC to assess the association with long term treatment response. In addition, for the most relevant models, the ROC curves (and the corresponding AUC) were compared.

### Results

Study Population: Table 1 provides an overview of the clinical characteristics of the study population. In total, 101 patients completed the study, for 83 patients nucleos(t)ide analog (NA) therapy was re-initiated due to relapse. One patient died, and one patient was diagnosed with hepatocellular carcinoma during the study period. The follow-up time after stop of treatment ranges from 38 to 814 days, with a mean and median period of follow-up of 482 and 637 days, respectively.

TABLE 1
Clinical characteristics of the study population

| Covariate | | |
|---|---|---|
| Age (years) | 50.8 ± 11.3 | |
| Gender | | |
| Male | 142 | (76.3%) |
| Female | 44 | (23.7%) |
| Previous NA therapy | | |
| Yes | 64 | (34.4%) |
| No | 122 | (65.6%) |
| Baseline HBeAg status | | |
| Negativity prior to start of | 142 | (76.3%) |
| NA therapy | | |
| Negativity obtained during | 44 | (23.7%) |
| NA therapy | | |
| Treatment regimen | | |
| Entecavir | 106 | (57.0%) |
| Tenofovir | 65 | (34.9%) |
| Other (Lamivudine, | 15 | (8.1%) |
| Telbivudine, Adefovir) | | |
| Length NA therapy (years) | 4.3 ± 1.9 | |
| On treatment viral suppression | | |
| No (HBV DNA all detected) | 26 | (14.0%) |
| Consistent (HBV DNA all | 38 | (20.4%) |
| not detected) | | |
| Inconsistent (HBV DNA both | 122 | (65.6%) |
| detected and not detected) | | |
| End of treatment HBV DNA | | |
| Detected | 96 | (51.6%) |
| Not detected | 90 | (48.4%) |
| End of treatment HBsAg | | |
| <100 IU/mL | 39 | (21.0%) |
| ≥100 IU/mL | 147 | (79.0%) |

| | |
|---|---|
| End of treatment HBsAg | 5.6 ± 2.3 |
| (log IU/mL) | |
| End of treatment HBsAb | 0.16 ± 0.73 |
| (log mIU/mL) | |
| End of treatment ALT (log U/L) | 3.1 ± 0.47 |
| End of treatment AST (log U/L) | 3.1 ± 0.29 |
| 1 month follow-up HBV DNA | 5.5 ± 5.0 |
| (log IU/mL) | |
| 1 month follow-up HBsAg | 5.5 ± 2.6 |
| (log IU/mL) | |
| 1 month follow-up ALT | 3.3 ± 0.79 |
| (log U/L) | |

Out of the 186 patients included in the cohort, 161 (86.6%) experienced a virological relapse (FIG. 1A), of whom 110 (59.2%) also had a clinical relapse (FIG. 1B). For 23 patients (12.4%), sustained clinical response was observed over the follow-up period (note that the patient who died and the patient diagnosed with hepatocellular carcinoma were excluded from the sustained clinical response group). 51 (27.42%) patients had a virological relapse without a clinical relapse. In the entire study period, 11 patients lost HBsAg. Out of those 11 HBsAg loss patients, 7 were also considered as sustained clinical responders. Three experienced a virological relapse before losing HBsAg, the fourth being the patient diagnosed with hepatocellular carcinoma and not considered a sustained clinical responder.

Clinical Relapse: The multivariate Cox proportional hazard regression analysis reveals that older age (HR, 1.02; 95% CI, 1.00-1.04; P=0.02), tenofovir treatment (compared to entecavir treatment) (HR, 1.87; 95% CI, 1.26-2.78; P=0.002), and high HBsAg at the end of treatment (HR, 3.24; 95% CI, 1.75-6.01; P<0.001), increase the hazard to clinical relapse and are therefore negatively associated to clinical relapse (Table 2a). An HBsAg level of 100 IU/mL was used as threshold to differentiate low and high end of treatment HBsAg, corresponding to 89.09% sensitivity and 35.53% specificity.

TABLE 2a
Cox proportional hazard regression analysis for clinical relapse

| | Univariate | | Multivariate | |
|---|---|---|---|---|
| | analysis | | analysis | |
| Covariate | HR (95% CI) | P value | HR (95% CI) | P value |
| Age (years) | 1.01 (0.99-1.02) | 0.43 | 1.02 (1.00-1.04) | 0.02 |
| Gender (male vs female) | 1.88 (1.15-3.09) | 0.01 | | |
| Previous NA therapy (yes vs no) | 1.26 (0.86-1.86) | 0.24 | | |
| Baseline HBeAg status (prior | 1.35 (0.85-2.14) | 0.2 | | |
| negativity vs during treatment) | | | | |
| Treatment regimen | | | | |
| Tenofovir vs entecavir | 1.77 (1.19-2.62) | 0.005 | 1.87 (1.26-2.78) | 0.004 |
| Other vs entecavir | 1.07 (0.51-2.25) | 0.85 | 1.21 (0.58-2.57) | 0.59 |
| Length NA therapy (years) | 0.96 (0.86-1.06) | 0.39 | | |
| On-treatment viral suppression | | | | |
| Inconsistent vs consistent | 1.46 (0.86-2.47) | 0.16 | | |
| No vs consistent | 1.71 (0.88-3.32) | 0.12 | | |
| HBV DNA at end of treatment | 1.39 (0.95-2.03) | 0.09 | | |
| (detected vs not detected) | | | | |
| HBsAg at end of treatment | 2.77 (1.51-5.05) | <0.001 | 3.24 (1.75-6.01) | <0.001 |
| (≥100 IU/mL vs <100 IU/mL) | | | | |
| HBsAb at end of treatment | 0.86 (0.63-1.18) | 0.35 | | |
| (log mIU/mL) | | | | |
| ALT at end of treatment (log U/L) | 1.04 (0.70-1.53) | 0.85 | | |
| AST at end of treatment (log U/L) | 1.02 (0.54-1.91) | 0.96 | | |
| HBV DNA at 1 month after stop | 1.21 (1.15-1.27) | <0.001 | | |
| treatment (log IU/mL) | | | | |
| ALT at 1 month after stop | 2.19 (1.60-3.01) | <0.001 | | |
| treatment (log U/L) | | | | |
| HBsAg at 1 month after stop | 1.21 (1.08-1.35) | <0.001 | | |
| treatment (log IU/mL) | | | | |

HBV DNA, HBsAg, and ALT at 1 month after stop treatment are additional variables showing a significant association to clinical relapse in the univariate models. These covariates were however excluded from the multivariate model as HBV DNA and ALT at 1 month after stop treatment are highly associated to the treatment regimen and HBsAg 1 month after stop treatment highly associated to end of treatment HBsAg (r=0.93, P<0.001). Only covariates measured during treatment were considered for the multivariate model to allow identification of a more predictive signature.

Among the 110 clinical relapsers, the onset of clinical relapse is on average 100 days later after stopping entecavir treatment compared to tenofovir treatment. The time to clinical relapse in the end of treatment (EOT) HBsAg high group is on average 60 days later compared to the low group. This is probably due to 7 of the 12 patients with low EOT HBsAg levels being in the tenofovir group (compared to 5 in the entecavir group).

Virological Relapse: The multivariate Cox proportional hazard regression analysis reveals that older age (HR, 1.02, 95% CI, 1.00-1.03; P=0.03), prior HBeAg negativity at the start of NA therapy (compared to acquiring HBeAg negativity during treatment) (HR, 2.20; 95% CI, 1.43-3.40; P<0.001), tenofovir treatment (compared to entecavir treatment) (HR, 2.97; 95% CI, 2.10-4.20; P<0.001), and high HBsAg at the end of treatment (HR, 2.37; 95% CI, 1.51-3.74; P<0.001), increase the hazard to virological relapse and are therefore negatively associated to virological relapse (Table 2b).

TABLE 2b
Cox proportional hazard regression analysis for virological relapse

| | Univariate | | Multivariate | | |
|---|---|---|---|---|---|
| | analysis | | analysis | | |
| Covariate | HR (95% CI) | P value | HR (95% CI) | | P value |
| Age (years) | 1.01 (1.00-1.03) | 0.05 | 1.02 | (1.00-1.03) | 0.03 |
| Gender (male vs female) | 1.53 (1.05-2.23) | 0.03 | | | |
| Previous NA therapy (yes vs no) | 1.64 (1.19-2.27) | 0.003 | | | |
| Baseline HBeAg status (prior | 2.02 (1.37-2.98) | <0.001 | 2.2 | (1.4-3.4) | 0.0003 |
| negativity vs during treatment) | | | | | |
| Treatment regimen | | | | | |
| Tenofovir vs entecavir | 2.49 (1.77-3.49) | <0.001 | 2.97 | (2.1-4.2) | <1e−5 |
| Other vs entecavir | 1.57 (0.88-2.81) | 0.13 | 1.6 | (1.6-0.9) | 0.11 |
| Length NA therapy (years) | 0.99 (0.91-1.07) | 0.79 | | | |
| On-treatment viral suppression | | | | | |
| Inconsistent vs consistent | 0.94 (0.63-1.41) | 0.77 | | | |
| No vs consistent | 1.17 (0.69-2.00) | 0.56 | | | |
| HBV DNA at end of treatment | 1.08 (0.79-1.47) | 0.64 | | | |
| (detected vs not detected) | | | | | |
| HBsAg at end of treatment | 1.64 (1.08-2.49) | 0.02 | 2.37 | (1.51-3.74) | 0.0002 |
| (≥100 IU/mL vs <100 IU/mL) | | | | | |
| HBsAb at end of treatment | 0.92 (0.73-1.16) | 0.49 | | | |
| (log mIU/mL) | | | | | |
| ALT at end of treatment (log U/L) | 1.09 (0.81-1.48) | 0.57 | | | |
| AST at end of treatment (log U/L) | 1.03 (0.62-1.70) | 0.93 | | | |
| HBV DNA at 1 month after stop | 1.21 (1.16-1.26) | <0.001 | | | |
| treatment (log IU/mL) | | | | | |
| ALT at 1 month after stop | 1.55 (1.23-1.97) | <0.001 | | | |
| treatment (log U/L) | | | | | |
| HBsAg at 1 month after stop | 1.08 (1.01-1.16) | 0.03 | | | |
| treatment (log IU/mL) | | | | | |

HBV DNA, HBsAg, and ALT at 1 month after treatment cessation were again excluded from the multivariate model due to strong association to the treatment regimen and end of treatment HBsAg. Finally, baseline HBeAg is associated to age (P<0.001) and gender (P=0.03), and therefore also excluded from the multivariate model. The effects of gender and age were moderate in a relatively unbalanced cohort, and HBeAg status is more reflecting the chronic stage of the patients.

Sustained Clinical Response: Logistic regression shows the negative association of prior HBeAg negativity at the start of NA therapy (OR, 0.25; 95% CI, 0.06-0.99; P=0.05), high HBsAg at the end of treatment (OR, 0.16; 95% CI, 0.04-0.55; P=0.0005), and higher HBV DNA 1 month after stop treatment (OR, 0.54; 95% CI, 0.28-0.78; P=0.01), to sustained clinical response (Table 2c).

TABLE 2c
Logistic regression analysis for sustained clinical response

| | Univariate analysis | | Multivariate analysis | | |
|---|---|---|---|---|---|
| Covariate | OR (95% CI) | P value | OR (95% CI) | | P value |
| Age (years) | 0.98 (0.94-1.01) | 0.21 | | | |
| Gender (male vs female) | 0.43 (0.17-1.10) | 0.07 | | | |
| Previous NA therapy (yes vs no) | 0.64 (0.22-1.63) | 0.37 | | | |
| Baseline HBeAg status (prior | 0.34 (0.14-0.87) | 0.02 | 0.25 | (0.06-0.99) | 0.05 |
| negativity vs during treatment) | | | | | |
| Treatment regimen | | | | | |
| Tenofovir vs entecavir | 0.79 (0.29-2.03) | 0.64 | | | |
| Other vs entecavir | 1.01 (0.15-4.20) | 0.99 | | | |
| Length NA therapy (years) | 1.01 (0.79-1.24) | 0.93 | | | |
| On-treatment viral suppression | | | | | |
| Inconsistent vs consistent | 0.81 (0.30-2.40) | 0.68 | | | |
| No vs consistent | 0.21 (0.01-1.36) | 0.16 | | | |
| HBV DNA at end of treatment | 0.69 (0.28-1.65) | 0.41 | | | |
| (detected vs not detected) | | | | | |
| HBsAg at end of treatment | 0.23 (0.09-0.57) | 0.001 | 0.16 | (0.04-0.55) | 0.005 |
| (≥100 IU/mL vs <100 IU/mL) | | | | | |
| HBsAb at end of treatment | 1.32 (0.78-2.04) | 0.22 | | | |
| (log mIU/mL) | | | | | |
| ALT at end of treatment (log U/L) | 1.40 (0.55-3.59) | 0.48 | | | |
| AST at end of treatment (log U/L) | 1.34 (0.29-5.79) | 0.7 | | | |
| HBV DNA at 1 month after stop | 0.53 (0.27-0.77) | 0.01 | 0.54 | (0.28-0.78) | 0.01 |
| treatment (log IU/mL) | | | | | |
| ALT at 1 month after stop | 0.59 (0.25-1.21) | 0.2 | | | |
| treatment (log U/L) | | | | | |
| HBsAg at 1 month after stop | 0.72 (0.59-0.86) | <0.001 | | | |
| treatment (log IU/mL) | | | | | |

Baseline HBeAg is associated to age (P<0.001) and gender (P=0.03), and therefore not included in the models including HLA markers. HBeAg status reflecting the chronic stage of the patients, this covariate was not included in the multivariate model. HBV DNA 1 month after treatment cessation is again excluded from the multivariate model as only covariates on-treatment were considered to obtain a more predictive signature.

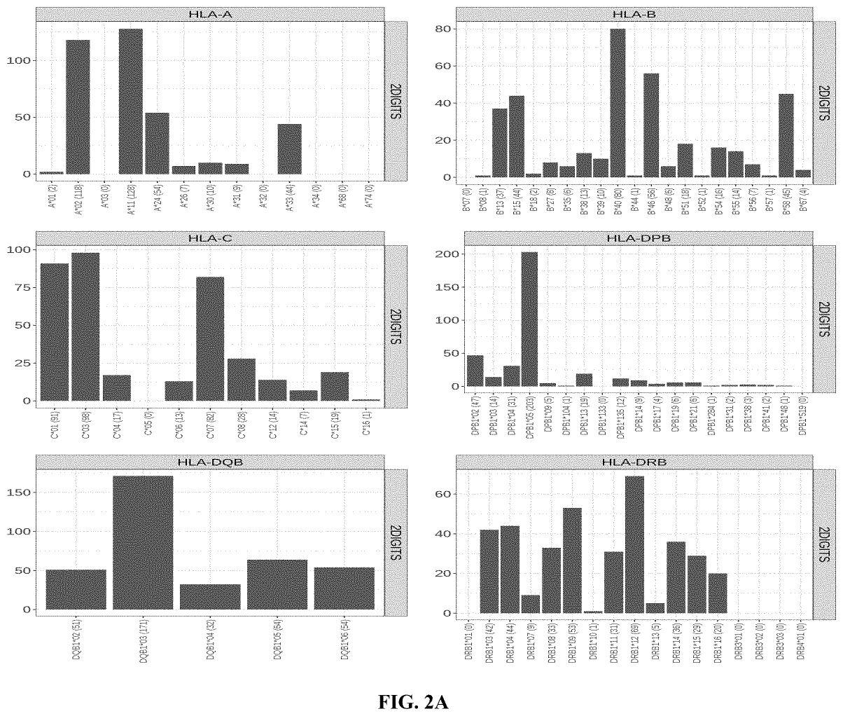

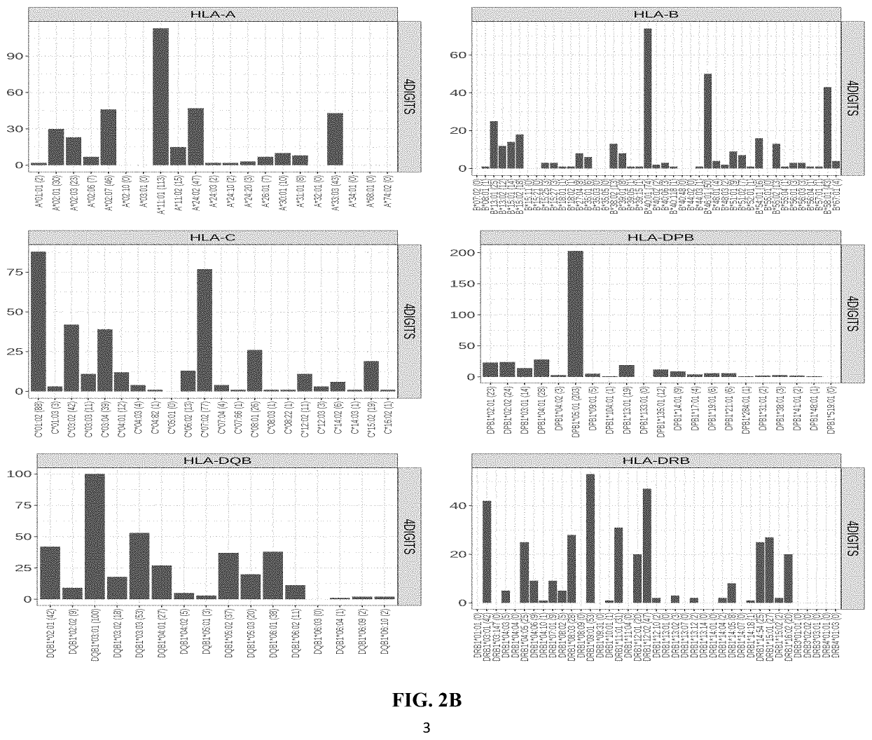

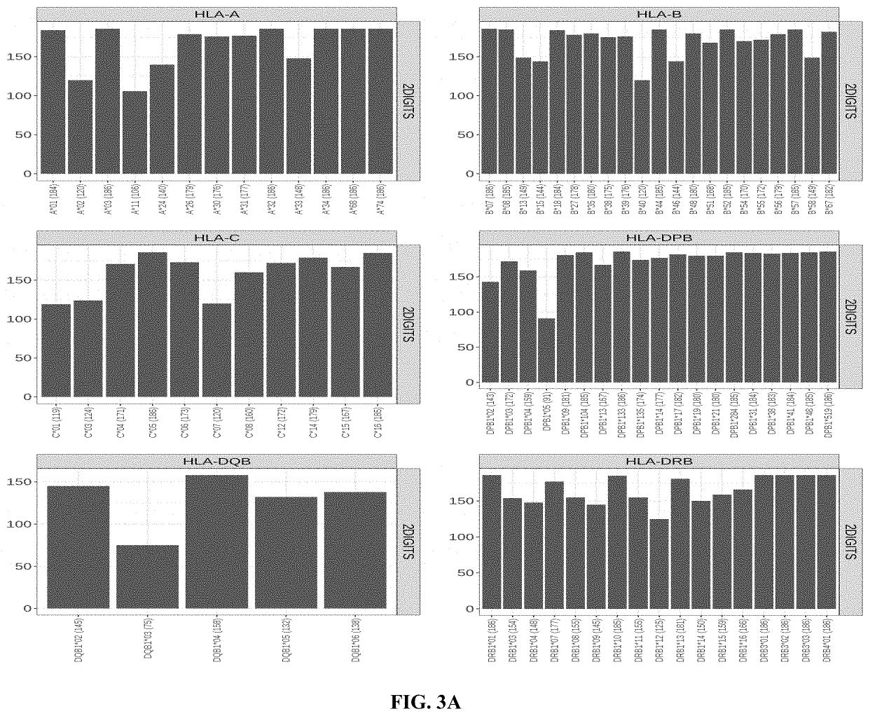

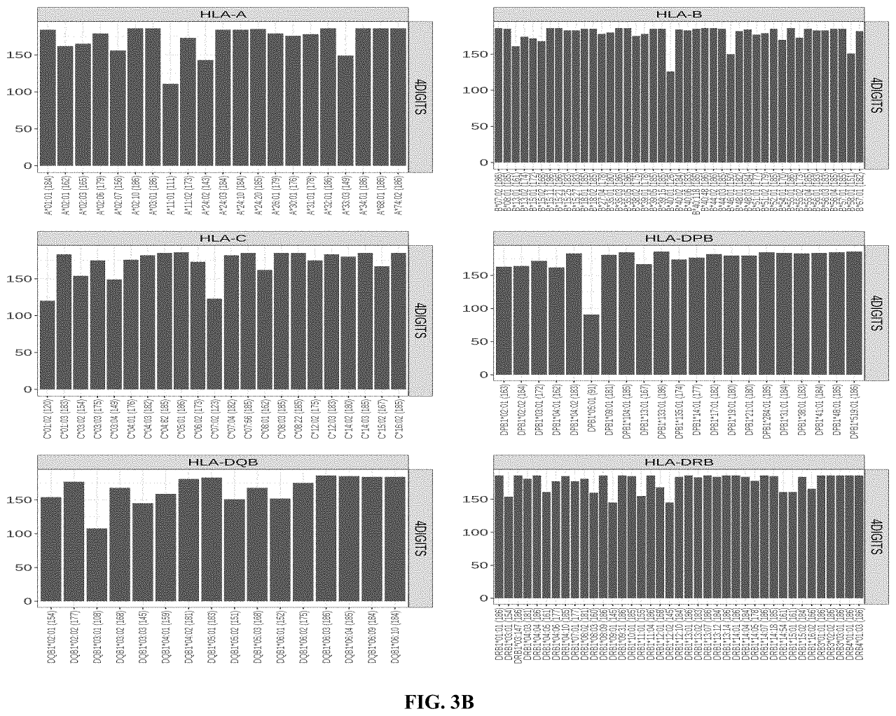

HLA alleles, homozygosity and HED score distribution: Overall frequency and distribution of different HLA alleles in this cohort is summarized on FIG. 2a (two digits resolution) and FIG. 2b (four digits resolution). Similarly, the overall frequency and distribution of homozygosity for each allele at each of the six loci is summarized on FIG. 3a (two digits resolution) and FIG. 3b (four digits resolution).

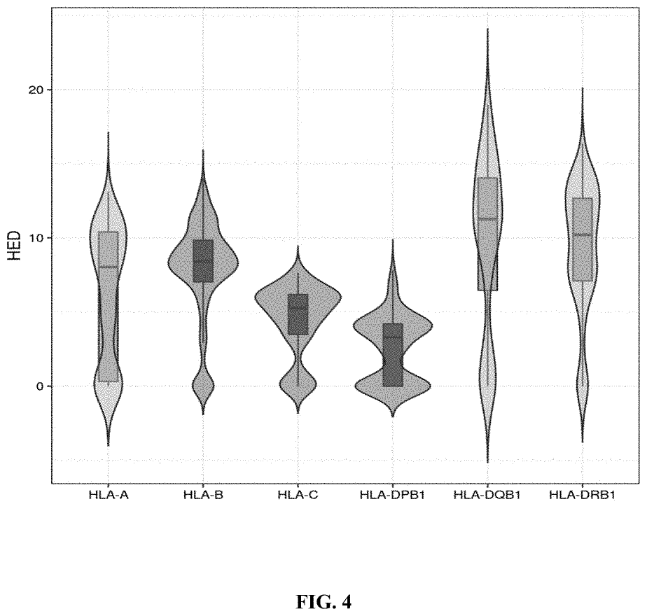

For each patient, at each locus and within each HLA region (class I and class II), a HED score per locus could be derived. The overall distribution of HED scores within this cohort per locus, is summarized in FIG. 4, showing a relatively higher HLA diversity for HLA-DQB1 and HLA-DRB1 regions. For each HLA region and for each analysis (onset of virological relapse, onset of clinical relapse, sustained clinical response), an optimal cut-off was defined. All cut-offs are summarized on Table 3, splitting the cohort in high HLA evolutionary diversity/low HLA evolutionary diversity. Table 3 demonstrates an overview of the HED thresholds obtained and the corresponding categorical variables derived. Non-missing HED scores>threshold are assigned High, and Low otherwise. One cut-off was derived for each HLA gene, in each of the three analyses performed (association with onset of virological relapse, onset of clinical relapse and sustained clinical response).

TABLE 3

| | Analysis/ | | | |
|---|---|---|---|---|
| Class | model | HLA gene | Variable | Threshold |
| Class I | CR | HLA-A | HED_HLA_A_CAT_CR | 3.88 |
| Class I | CR | HLA-B | HED_HLA_B_CAT_CR | 9.38 |
| Class I | CR | HLA-C | HED_HLA_C_CAT_CR | 0.00 |
| Class I | CR | MEAN_MHCI | HED_MEANI_CAT_CR | 6.35 |
| Class I | VR | HLA-A | HED_HLA_A_CAT_VR | 3.88 |
| Class I | VR | HLA-B | HED_HLA_B_CAT_VR | 9.37 |
| Class I | VR | HLA-C | HED_HLA_C_CAT_VR | 0.00 |
| Class I | VR | MEAN_MHCI | HED_MEANI_CAT_VR | 8.69 |
| Class I | SCR | HLA-A | HED_HLA_A_CAT_SCR | 4.85 |
| Class I | SCR | HLA-B | HED_HLA_B_CAT_SCR | 9.38 |
| Class I | SCR | HLA-C | HED_HLA_C_CAT_SCR | 7.37 |
| Class I | SCR | MEAN_MHCI | HED_MEANI_CAT_SCR | 7.61 |
| Class II | CR | HLA-DPB1 | HED_HLA_P_CAT_CR | 4.15 |
| Class II | CR | HLA-DQB1 | HED_HLA_Q_CAT_CR | 11.72 |
| Class II | CR | HLA-DRB1 | HED_HLA_R_CAT_CR | 12.68 |
| Class II | CR | MEAN_MHCII | HED_MEANII_CAT_CR | 7.67 |
| Class II | VR | HLA-DPB1 | HED_HLA_P_CAT_VR | 2.21 |
| Class II | VR | HLA-DQB1 | HED_HLA_Q_CAT_VR | 11.98 |
| Class II | VR | HLA-DRB1 | HED_HLA_R_CAT_VR | 12.68 |
| Class II | VR | MEAN_MHCII | HED_MEANII_CAT_VR | 7.55 |
| Class II | SCR | HLA-DPB1 | HED_HLA_P_CAT_SCR | 2.43 |
| Class II | SCR | HLA-DQB1 | HED_HLA_Q_CAT_SCR | 11.72 |
| Class II | SCR | HLA-DRB1 | HED_HLA_R_CAT_SCR | 14.16 |
| Class II | SCR | MEAN_MHCII | HED_MEANII_CAT_SCR | 8.21 |

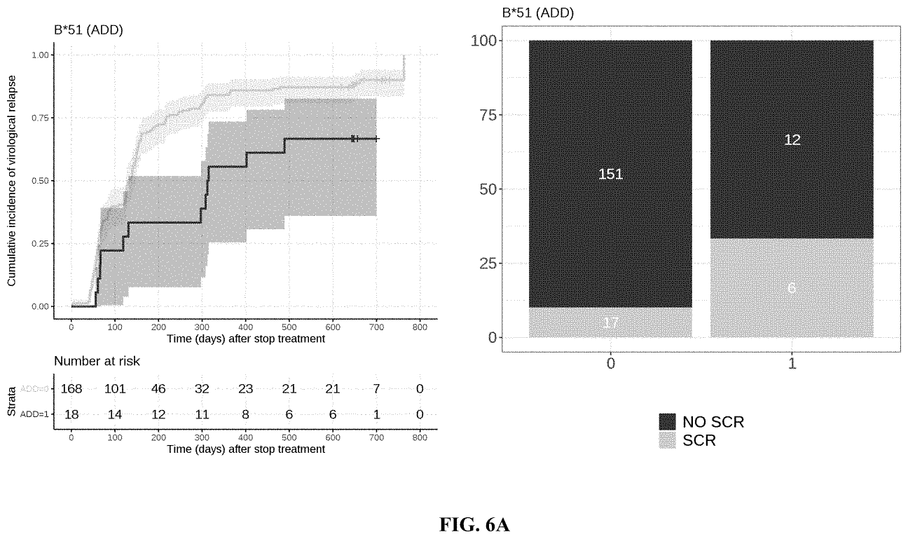

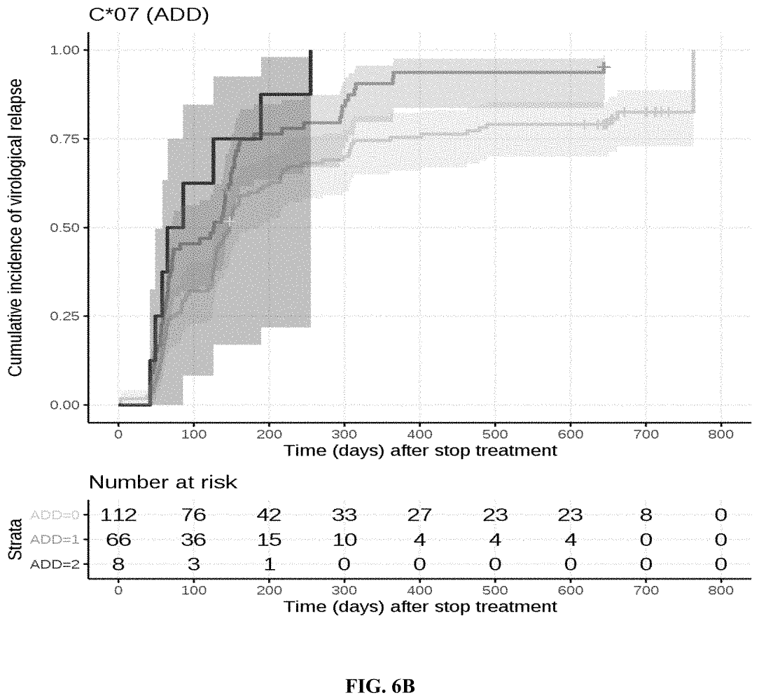

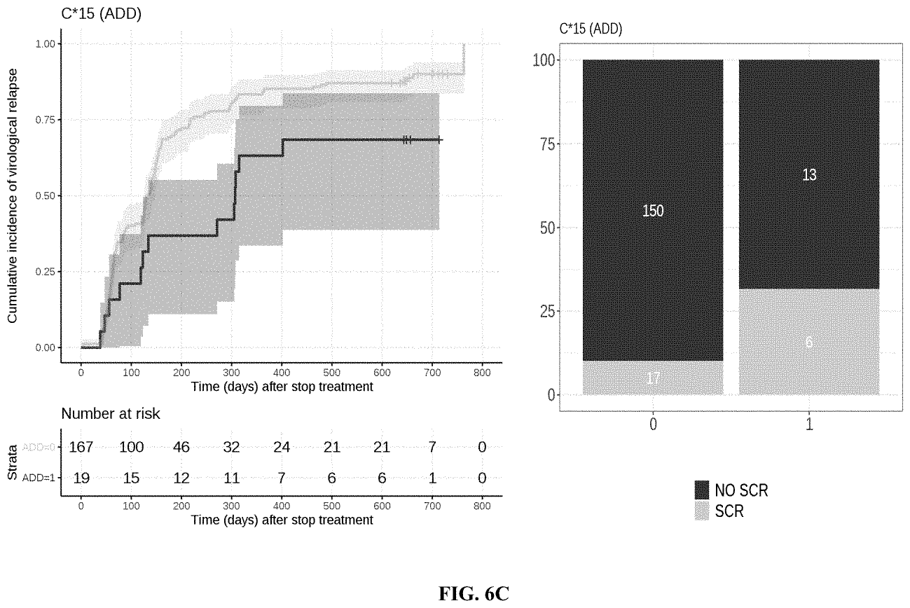

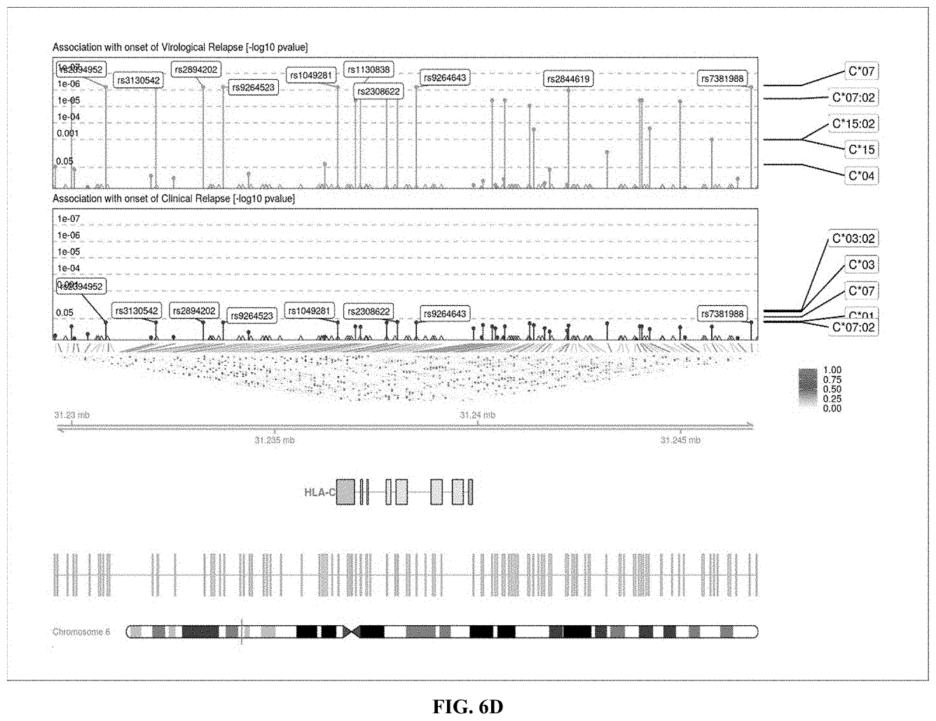

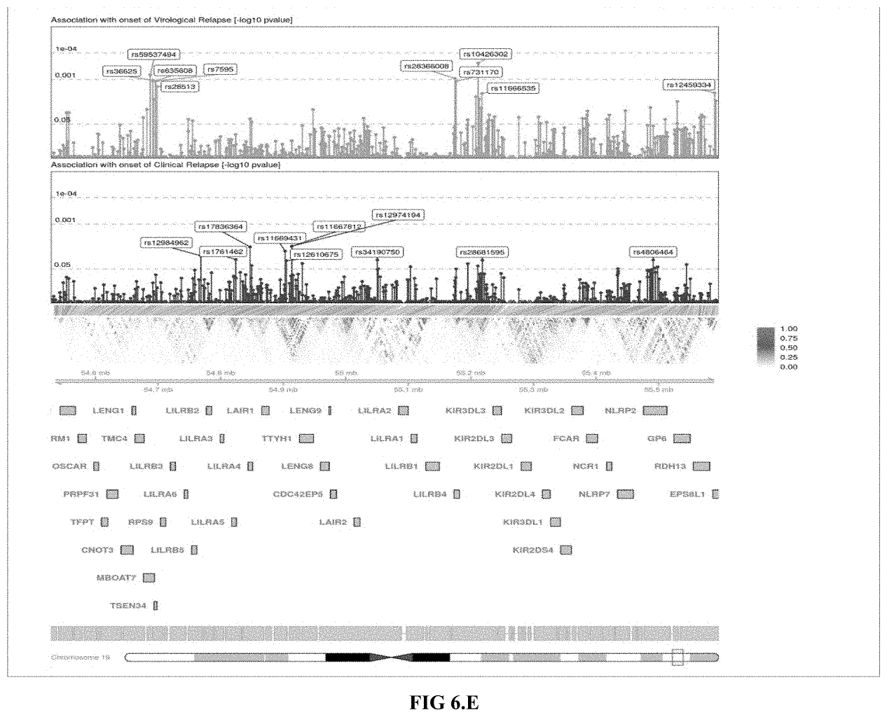

HLA alleles associated with onset of relapse and sustained clinical response: Interestingly, several HLA alleles in the HLA class I region showed a significant association with onset of virological relapse or sustained clinical response, in HLA-B and HLA-C loci (more B*51 alleles, less C*07:02 alleles and more C*15:02 alleles being protective against virological relapse) (Table 4, FIGS. 6A-D).

HLA alleles (two digits resolution) are significantly associated with onset of virological relapse and/or sustained clinical response. As indicated in FIGS. 6A-6C, Kaplan-Meier curves of B*51 (A), C*07 (B) and C*15 (C) alleles are associated with onset of virological relapse, and barplots represent the distribution of presence and absence of B*51 alleles (A) (FIG. 6A) and C*15 alleles (C) (FIG. 6C) within sustained clinical responders.

As indicated in FIG. 6D, Single Nucleotide Polymorphisms (SNPs) in the HLA-C region showed similar level of significance (p-value<1e-6) while testing association with onset of virological or clinical relapse as a candidate approach. The plot of p-values is relative to genomic positions from the GWAS association analysis on onset of clinical relapse (top panel) or onset of viral relapse (2nd panel). Each point corresponds to the p-value of a SNP. The labels indicate the RS identifiers for the SNP corresponding to the lowest p-value obtained. The middle panel shows the linkage disequilibrium between pairs of SNPs using the r2 as metric. The bottom panel provides an overview of the genes contained in the genomic region spanning the genes (+/−7000 bp). The genes span the region as well as the coverage of the SNPs on the axiom chip (bottom track) and the position on the chromosome. The lines and labels on the right side of the panel show the p-values of the test on the HLA alleles, following the genotyping of the loci. The labels correspond to the 5 alleles with the lowest p-values obtained.

As HLA-C protein interact with KIR and LILR ligands on Natural Killer cells, SNP on the Leukocyte Receptor Complex (LRC) region have been explored (FIG. 6, panel E) showing a significant association (p-value<0.05) in LILRB4 LILRA4 and LILRB5 genes and KIR3DL1, KIR2DL4, KIR2DL2 and KIR2DL3 genes (Table 4c, FIG. 6E). FIG. 6E represents the association between SNPs in the leukocyte receptor complex (LRC region is defined in (GRCh37.p13): CHR 19 54528888-55595686) including KIR ligands and onset of virological and clinical relapse. The plot of p-values is relative to genomic positions from the GWAS association analysis on onset of clinical relapse (top panel) or onset of viral relapse (2nd panel). Each point corresponds to the p-value of a SNP. The labels indicate the RS identifiers for the SNP corresponding to the 10 lowest p-values. The middle panel shows the linkage disequilibrium between pairs of SNPs using the r2 as metric. The bottom panel provides an overview of the genes contained in the genomic region 19:54528888-55595686. The genes span the region as well as the coverage of the SNPs on the axiom chip (bottom track) and the position on the chromosome.

Table 4a demonstrates an overview of the HLA alleles significantly associated with onset of virological relapse or sustained clinical relapse after multiple testing correction (False Discover rate <10%). For the association with onset of relapse both HBsAg levels at the last visit on treatment and the last treatment regimen were taken into account as main effects, for testing association with sustained clinical response, HBsAg levels at the last visit on treatment was taken into account as main effect. Table 4b demonstrates an overview of the SNPs in the HLA-C region significantly (unadjusted p-value<0.05) associated with onset of relapse (treatment regimen being considered as main effect). Table 4c demonstrates an overview of the SNPs in the LRC region significantly (unadjusted p-value<0.05) associated with onset of relapse (treatment regimen being considered as main effect).

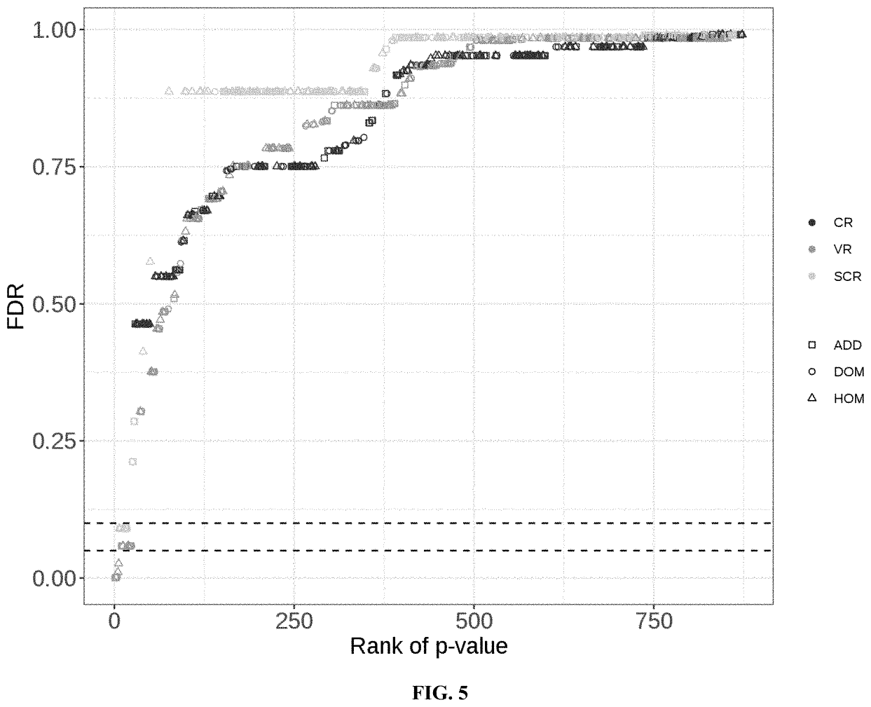

No significant HLA allelic association with onset of clinical relapse could be observed in this cohort and no significant association between HLA homozygosity and onset of relapse or sustained clinical response could be observed after multiple testing corrections (FIG. 5).

TABLE 4a

| ALLELE | LOCUS | DIGITS | MAF | SINGLE_F | ANALYSIS | term | estimate | p.value | FDR | PROTECTIVE |
|---|---|---|---|---|---|---|---|---|---|---|
| B*51 | HLA-B | 2DIGITS | 0.10 | FALSE | VR | ADD | −0.91 | 0.00 | 0.06 | more |
| B*51 | HLA-B | 2DIGITS | 0.10 | FALSE | SCR | ADD | 1.91 | 0.00 | 0.09 | more |
| C*07 | HLA-C | 2DIGITS | 0.40 | FALSE | VR | ADD | 0.70 | 0.00 | 0.00 | less |
| C*07:02 | HLA-C | 4DIGITS | 0.38 | FALSE | VR | ADD | 0.65 | 0.00 | 0.00 | less |
| C*15 | HLA-C | 2DIGITS | 0.10 | FALSE | VR | ADD | −0.87 | 0.00 | 0.06 | more |
| C*15 | HLA-C | 2DIGITS | 0.10 | FALSE | SCR | ADD | 1.83 | 0.00 | 0.09 | more |
| C*15:02 | HLA-C | 4DIGITS | 0.10 | FALSE | VR | ADD | −0.87 | 0.00 | 0.06 | more |
| C*15:02 | HLA-C | 4DIGITS | 0.10 | FALSE | SCR | ADD | 1.83 | 0.00 | 0.09 | more |

TABLE 4b

| SNP | RSID | Gene | CHR | POS | Estimate | P-VALUE | Response | Origin | Al | A2 | MAF | Risk Allele | Prot. Allele |
|---|---|---|---|---|---|---|---|---|---|---|---|---|---|
| AX-11389327 | rs2394952 | HLA-C | 6 | 31230882 | 0.728044 | 6.66390146064622e-7 | Viral Relapse | MHC Candidate Top | G | A | 0.2225 | G | A |
| AX-11435438 | rs3130542 | HLA-C | 6 | 31232111 | 0.728044 | 6.66390146064622e−7 | Viral Relapse | MHC Candidate Top | A | G | 0.2225 | A | G |
| AX-11425415 | rs2894202 | HLA-C | 6 | 31233270 | 0.728044 | 6.66390146064622e−7 | Viral Relapse | MHC Candidate Top | T | G | 0.2225 | T | G |
| AX-15354727 | rs9264523 | HLA-C | 6 | 31233758 | 0.728044 | 6.66390146064622e−7 | Viral Relapse | MHC Candidate Top | T | A | 0.2225 | T | A |
| AX-11110708 | rs1049281 | HLA-C | 6 | 31236567 | 0.728044 | 6.66390146064622e−7 | Viral Relapse | MHC Candidate Top | T | C | 0.2225 | T | C |
| AX-35731691 | rs9264643 | HLA-C | 6 | 31238492 | 0.728044 | 6.66390146064622e−7 | Viral Relapse | MHC Candidate Top | C | A | 0.2225 | C | A |
| AX-83069277 | rs1130838 | HLA-C | 6 | 31237124 | 0.774919 | 7.26418708851817e−7 | Viral Relapse | MHC Candidate Top | T | C | 0.2139 | T | C |
| AX-11389316 | rs2394888 | HLA-C | 6 | 31202246 | 0.706182 | 1.02E-06 | Viral Relapse | MHC Candidate Top | A | G | 0.217 | A | G |
| AX-83089411 | | HLA-C | 6 | 31237768 | 0.710822 | 1.16E-06 | Viral Relapse | MHC Candidate Top | AGC | T | 0.2225 | AGC | T |
| AX-83033597 | rs2308622 | HLA-C | 6 | 31238029 | 0.815332 | 1.7E-06 | Viral Relapse | MHC Candidate Top | T | C | 0.2056 | T | C |
| AX-11676212 | rs9264416 | HLA-C | 6 | 31230042 | 0.676271 | 4.21E-06 | Viral Relapse | MHC Candidate Top | G | A | 0.2115 | G | A |
| AX-41951051 | rs2001181 | HLA-C | 6 | 31236998 | 0.676271 | 4.21E-06 | Viral Relapse | MHC Candidate Top | C | T | 0.2115 | C | T |
| AX-11435692 | rs3132499 | HLA-C | 6 | 31207920 | 0.669243 | 5.54E-06 | Viral Relapse | MHC Candidate Top | C | T | 0.2088 | C | T |
| AX-11435436 | rs3130532 | HLA-C | 6 | 31208453 | 0.669243 | 5.54E-06 | Viral Relapse | MHC Candidate Top | A | G | 0.2088 | A | G |
| AX-15354622 | rs3130941 | HLA-C | 6 | 31197514 | 0.662868 | 7.75E-06 | Viral Relapse | MHC Candidate Top | C | G | 0.2072 | C | G |
| AX-11435434 | rs3130528 | HLA-C | 6 | 31199460 | 0.598808 | 1.66E-05 | Viral Relapse | MHC Candidate Top | T | C | 0.2115 | T | C |
| AX-11435919 | rs3134782 | HLA-C | 6 | 31197633 | 0.573613 | 1.68E-05 | Viral Relapse | MHC Candidate Top | G | A | 0.221 | G | A |
| AX-11435916 | rs3134769 | HLA-C | 6 | 31205754 | 0.5054 | 7.72E-05 | Viral Relapse | MHC Candidate Top | T | C | 0.3324 | T | C |
| AX-11435432 | rs3130521 | HLA-C | 6 | 31196376 | 0.510141 | 8.18E-05 | Viral Relapse | MHC Candidate Top | T | C | 0.3287 | T | C |
| AX-82884445 | rs3130695 | HLA-C | 6 | 31211050 | 0.470284 | 0.000258 | Viral Relapse | MHC Candidate Top | A | G | 0.3242 | A | G |
| AX-15354633 | rs3130685 | HLA-C | 6 | 31206206 | −0.42529 | 0.00026 | Viral Relapse | MHC Candidate Top | T | C | 0.4973 | C | T |
| AX-15354698 | rs2524119 | HLA-C | 6 | 31229404 | 0.380815 | 0.000772 | Viral Relapse | MHC Candidate Top | T | C | 0.2869 | T | C |
| AX-11435433 | rs3130527 | HLA-C | 6 | 31198987 | 0.389965 | 0.003003 | Viral Relapse | MHC Candidate Top | T | C | 0.2967 | T | C |
| AX-11425411 | rs2894186 | HLA-C | 6 | 31206868 | −0.31881 | 0.008845 | Viral Relapse | MHC Candidate Top | C | G | 0.4011 | G | C |
| AX-50495706 | rs3130439 | HLA-C | 6 | 31221023 | −0.23878 | 0.015167 | Viral Relapse | MHC Candidate Top | A | G | 0.5 | G | A |
| AX-11433206 | rs3095254 | HLA-C | 6 | 31221668 | 0.27173 | 0.01963 | Viral Relapse | MHC Candidate Top | C | G | 0.3591 | C | G |
| AX-11676201 | rs9264189 | HLA-C | 6 | 31220163 | 0.249459 | 0.02553 | Viral Relapse | MHC Candidate Top | A | C | 0.4282 | A | C |
| AX-11389323 | rs2394943 | HLA-C | 6 | 31220388 | 0.247638 | 0.025585 | Viral Relapse | MHC Candidate Top | T | C | 0.4313 | T | C |
| AX-11685437 | rs9394047 | HLA-C | 6 | 31236250 | −0.27896 | 0.031231 | Viral Relapse | MHC Candidate Top | C | A | 0.2652 | A | C |
| AX-11435538 | rs3130948 | HLA-C | 6 | 31193523 | −0.21961 | 0.032006 | Viral Relapse | MHC Candidate Top | T | C | 0.3785 | C | T |
| AX-15354704 | rs9368666 | HLA-C | 6 | 31229644 | −0.24909 | 0.045811 | Viral Relapse | MHC Candidate Top | G | A | 0.275 | A | G |
| AX-11435536 | rs3130942 | HLA-C | 6 | 31197293 | −0.25822 | 0.047561 | Viral Relapse | MHC Candidate Top | T | C | 0.3956 | C | T |
| AX-11435471 | rs3130688 | HLA-C | 6 | 31210216 | 0.251956 | 0.049806 | Viral Relapse | MHC Candidate Top | C | T | 0.4203 | C | T |
| AX-11417845 | rs3130536 | HLA-C | 6 | 31210532 | 0.251956 | 0.049806 | Viral Relapse | MHC Candidate Top | A | G | 0.4203 | A | G |
| AX-11435914 | rs3134750 | HLA-C | 6 | 31218087 | 0.251956 | 0.049806 | Viral Relapse | MHC Candidate Top | A | G | 0.4203 | A | G |
| AX-11490436 | rs4084090 | HLA-C | 6 | 31218835 | −0.50926 | 0.029462 | Clinical Relapse | MHC Candidate Top | G | A | 0.1181 | A | G |
| AX-35731063 | rs9264127 | HLA-C | 6 | 31211592 | −0.5527 | 0.030238 | Clinical Relapse | MHC Candidate Top | G | C | 0.1023 | C | G |
| AX-11676190 | rs9264039 | HLA-C | 6 | 31196591 | −0.47866 | 0.040352 | Clinical Relapse | MHC Candidate Top | A | G | 0.1154 | G | A |
| AX-11485919 | rs3868078 | HLA-C | 6 | 31199164 | −0.47866 | 0.040352 | Clinical Relapse | MHC Candidate Top | T | C | 0.1154 | C | T |

TABLE 4c

| SNP | RSID | Gene | CHR | POS | Estimate |
|---|---|---|---|---|---|
| AX-40474401 | rs10426302 | — | 19 | 55211547 | 0.451712561 |
| AX-32791043 | rs59537494 | MBOAT7 | 19 | 54688378 | 0.476837708 |
| AX-32793765 | rs28366008 | LILRB4 | 19 | 55174498 | −0.513992692 |
| AX-32791067 | rs36625 | MBOAT7 | 19 | 54692523 | 0.355084386 |
| AX-11562447 | rs635608 | TSEN34 | 19 | 54696488 | 0.344914709 |
| AX-40473181 | rs7595 | TSEN34 | 19 | 54697079 | 0.344914709 |
| AX-83323675 | rs731170 | LILRB4 | 19 | 55176262 | −0.468562637 |
| AX-13455041 | rs28513 | — | 19 | 54699386 | 0.366160334 |
| AX-32795451 | rs12459334 | EPS8L1 | 19 | 55588726 | 0.533351627 |
| AX-51295385 | rs11666535 | — | 19 | 55217660 | 0.410213441 |
| AX-11527755 | rs4806807 | — | 19 | 55207900 | 0.359721702 |
| AX-40474407 | rs11084367 | — | 19 | 55213281 | 0.323708459 |
| AX-40473175 | rs39714 | MBOAT7 | 19 | 54693682 | 0.295723304 |
| AX-32795469 | rs1654474 | EPS8L1 | 19 | 55590594 | 0.496196364 |
| AX-11207928 | rs12462907 | GP6 | 19 | 55529100 | 0.24216561 |
| AX-11643598 | rs775893 | NLRP2 | 19 | 55486167 | −0.290614208 |
| AX-12388453 | rs10416527 | — | 19 | 55215632 | 0.287752966 |
| AX-11505772 | rs4441391 | — | 19 | 55216681 | 0.287752966 |
| AX-40473161 | rs40167 | MBOAT7 | 19 | 54682975 | 0.268925659 |
| AX-32792529 | rs11084339 | TTYH1 | 19 | 54948086 | −0.444904956 |
| AX-40474775 | rs775875 | NLRP7 | 19 | 55446522 | 0.262662903 |
| AX-40474665 | rs2304225 | FCAR | 19 | 55385962 | 0.374671188 |
| AX-13454965 | rs4077076 | VSTM1 | 19 | 54558412 | −0.286459718 |
| AX-40472901 | rs4442924 | VSTM1 | 19 | 54554942 | 0.356012339 |
| AX-11527741 | rs4806527 | KIR3DL1 | 19 | 55228948 | −0.254093802 |
| AX-83135617 | rs12608979 | RDH13 | 19 | 55568084 | 0.377246744 |
| AX-11479003 | rs3765013 COSV52557220 | NCR1 | 19 | 55420801 | −0.242495817 |
| AX-40474663 | rs12462181 | — | 19 | 55385435 | 0.288971856 |
| AX-13455616 | rs2075731 COSV52550169 | KIR3DL3 | 19 | 55237616 | 0.258917784 |
| AX-32795291 | rs12608988 | RDH13 | 19 | 55567979 | 0.366644765 |
| AX-94351577 | rs190480734 | KIR2DL4 | 19 | 55307719 | 0.1850665 |
| AX-40475037 | rs1654452 | RDH13 | 19 | 55572284 | 0.322538925 |
| AX-32794129 | rs11879415 | KIR2DL4 | 19 | 55276380 | −0.260151598 |
| AX-40473143 | rs653560 | TMC4 | 19 | 54677103 | −0.246315381 |
| AX-40475023 | rs11084387 | RDH13 | 19 | 55565352 | 0.359882921 |
| AX-11166815 | rs11672111 | RDH13 | 19 | 55565634 | 0.359882921 |
| AX-40475027 | rs10424969 | RDH13 | 19 | 55566512 | 0.359882921 |
| AX-32792987 | rs77389424 | LAIR2 | 19 | 55028970 | 0.348600312 |
| AX-11477710 | rs3745902 CM1111041 | KIR3DL2 | 19 | 55378008 | 0.275803142 |
| AX-11166876 | rs11672983 | — | 19 | 55383051 | 0.275803142 |
| AX-40473637 | rs17836364 | LILRA4 | 19 | 54847587 | −0.310174266 |
| AX-32794537 | rs34549987 | NCR1 | 19 | 55416954 | −0.226798441 |
| AX-40473405 | rs11668526 | RPS9 | 19 | 54749060 | −0.320096266 |
| AX-11166583 | rs11667105 | NLRP2 | 19 | 55509865 | 0.215814943 |
| AX-40474339 | rs1749282 | — | 19 | 55194821 | −0.238413105 |
| AX-11280328 | rs1654660 | — | 19 | 55195187 | −0.238413105 |
| AX-13455605 | rs73618328 | KIR3DL1 | 19 | 55228122 | −0.237775016 |
| AX-32794029 | rs270785 | KIR3DL1 | 19 | 55233850 | 0.315362658 |
| AX-83521570 | rs76522818 | LILRB5 | 19 | 54755918 | 0.410121214 |
| AX-94364811 | rs62131745 | KIR3DL1 | 19 | 55234854 | −0.228255819 |
| AX-40473651 | rs2241384 | LILRA4 | 19 | 54849942 | 0.275552297 |
| AX-12454687 | rs1325158 | KIR3DL1 | 19 | 55226402 | 0.238167326 |
| AX-11112044 | rs10500318 | KIR2DL4 | 19 | 55320779 | 0.333357496 |
| AX-11482916 | rs3816051 | — | 19 | 55385604 | 0.227606755 |
| AX-32794205 | rs34508934 | KIR2DL4 | 19 | 55311067 | 0.311133039 |
| AX-13455932 | rs12460627 | GP6 | 19 | 55529089 | 0.231413661 |
| AX-40473767 | rs11667812 | TTYH1 | 19 | 54913994 | 0.36934225 |
| AX-13455223 | rs12974194 | TTYH1 | 19 | 54914134 | 0.36934225 |
| AX-40473637 | rs17836364 | LILRA4 | 19 | 54847587 | −0.478999864 |
| AX-40473755 | rs11669431 | TTYH1 | 19 | 54903416 | −0.356897282 |
| AX-11234805 | rs12984962 | — | 19 | 54769205 | −0.611762362 |
| AX-32791879 | rs1761462 | LILRA4 | 19 | 54824667 | −0.400809784 |
| AX-32794831 | rs4806464 | NLRP2 | 19 | 55490720 | −0.436542373 |
| AX-11442739 | rs34190750 | — | 19 | 55050562 | 0.308344304 |
| AX-32793975 | rs28681595 | LILRP2 | 19 | 55218126 | 0.353842095 |
| AX-94348052 | rs12610675 | TTYH1 | 19 | 54905393 | 0.29328675 |
| AX-32792425 | rs12463051 | TTYH1 | 19 | 54930952 | −0.315289374 |
| AX-40474339 | rs1749282 | — | 19 | 55194821 | −0.303231998 |
| AX-11280328 | rs1654660 | — | 19 | 55195187 | −0.303231998 |
| AX-32795157 | rs41275824 | GP6 | 19 | 55543973 | 0.284695244 |
| AX-40473651 | rs2241384 | LILRA4 | 19 | 54849942 | 0.342303097 |
| AX-11234761 | rs12983338 | — | 19 | 55210931 | 0.352021106 |
| AX-40474129 | rs272408 | LILRB1 | 19 | 55103603 | 0.328062285 |
| AX-40474859 | rs10412569 | NLRP2 | 19 | 55497855 | −0.352993676 |
| AX-32794851 | | NLRP2 | 19 | 55493651 | −0.352120385 |
| AX-13455842 | rs622941 | NLRP7 | 19 | 55434458 | −0.265313309 |
| AX-13455332 | rs60690598 | — | 19 | 55052198 | 0.297260211 |
| SNP | P-VALUE | Response | MAP | Risk Allele | Protective Allele |
| AX-40474401 | 0.000240387 | Viral Relapse | 0.3654 | G | A |
| AX-32791043 | 0.000678068 | Viral Relapse | 0.2198 | G | C |
| AX-32793765 | 0.000925382 | Viral Relapse | 0.1519 | T | C |
| AX-32791067 | 0.001107075 | Viral Relapse | 0.4011 | A | G |
| AX-11562447 | 0.001176932 | Viral Relapse | 0.4643 | C | T |
| AX-40473181 | 0.001176932 | Viral Relapse | 0.4643 | T | C |
| AX-83323675 | 0.001187372 | Viral Relapse | 0.239 | G | A |
| AX-13455041 | 0.001868607 | Viral Relapse | 0.4973 | C | A |
| AX-32795451 | 0.003247705 | Viral Relapse | 0.1016 | C | T |
| AX-51295385 | 0.003485518 | Viral Relapse | 0.2639 | G | T |
| AX-11527755 | 0.004576148 | Viral Relapse | 0.3407 | G | T |
| AX-40474407 | 0.005596541 | Viral Relapse | 0.4973 | G | A |
| AX-40473175 | 0.005852794 | Viral Relapse | 0.4536 | C | G |
| AX-32795469 | 0.006516564 | Viral Relapse | 0.1016 | T | C |
| AX-11207928 | 0.006911051 | Viral Relapse | 0.306 | T | C |
| AX-11643598 | 0.011613591 | Viral Relapse | 0.3352 | C | G |
| AX-12388453 | 0.012488897 | Viral Relapse | 0.4835 | G | A |
| AX-11505772 | 0.012488897 | Viral Relapse | 0.4835 | G | A |
| AX-40473161 | 0.013809827 | Viral Relapse | 0.4836 | G | A |
| AX-32792529 | 0.014154957 | Viral Relapse | 0.1566 | G | A |
| AX-40474775 | 0.015424157 | Viral Relapse | 0.478 | T | A |
| AX-40474665 | 0.016022686 | Viral Relapse | 0.1806 | G | C |
| AX-13454965 | 0.018143745 | Viral Relapse | 0.2374 | G | A |
| AX-40472901 | 0.018388072 | Viral Relapse | 0.1951 | C | A |
| AX-11527741 | 0.022274657 | Viral Relapse | 0.4836 | G | A |
| AX-83135617 | 0.022286121 | Viral Relapse | 0.1519 | T | C |
| AX-11479003 | 0.022506041 | Viral Relapse | 0.467 | C | A |
| AX-40474663 | 0.022787314 | Viral Relapse | 0.3022 | C | T |
| AX-13455616 | 0.02605448 | Viral Relapse | 0.4062 | C | T |
| AX-32795291 | 0.026168426 | Viral Relapse | 0.1511 | A | G |
| AX-94351577 | 0.026403665 | Viral Relapse | 0.3825 | A | G |
| AX-40475037 | 0.026843638 | Viral Relapse | 0.1841 | A | G |
| AX-32794129 | 0.027476482 | Viral Relapse | 0.3861 | C | T |
| AX-40473143 | 0.028071892 | Viral Relapse | 0.4725 | C | G |
| AX-40475023 | 0.028445048 | Viral Relapse | 0.1538 | A | G |
| AX-11166815 | 0.028445048 | Viral Relapse | 0.1538 | C | G |
| AX-40475027 | 0.028445048 | Viral Relapse | 0.1538 | G | T |
| AX-32792987 | 0.030102735 | Viral Relapse | 0.1243 | C | T |
| AX-11477710 | 0.030573053 | Viral Relapse | 0.2995 | T | C |
| AX-11166876 | 0.030573053 | Viral Relapse | 0.2995 | A | G |
| AX-40473637 | 0.031362692 | Viral Relapse | 0.2238 | G | A |
| AX-32794537 | 0.032477939 | Viral Relapse | 0.5 | T | C |
| AX-40473405 | 0.032759469 | Viral Relapse | 0.2363 | C | T |
| AX-11166583 | 0.03295294 | Viral Relapse | 0.2857 | A | G |
| AX-40474339 | 0.034679844 | Viral Relapse | 0.3654 | A | G |
| AX-11280328 | 0.034679844 | Viral Relapse | 0.3654 | G | A |
| AX-13455605 | 0.035164827 | Viral Relapse | 0.4808 | T | C |
| AX-32794029 | 0.035843002 | Viral Relapse | 0.239 | T | C |
| AX-83521570 | 0.037299895 | Viral Relapse | 0.1126 | G | C |
| AX-94364811 | 0.038842978 | Viral Relapse | 0.4973 | T | C |
| AX-40473651 | 0.040517637 | Viral Relapse | 0.2308 | A | G |
| AX-12454687 | 0.041514617 | Viral Relapse | 0.4093 | C | T |
| AX-11112044 | 0.042556048 | Viral Relapse | 0.1484 | A | G |
| AX-11482916 | 0.046660994 | Viral Relapse | 0.3674 | C | T |
| AX-32794205 | 0.047553752 | Viral Relapse | 0.1456 | A | C |
| AX-13455932 | 0.047739197 | Viral Relapse | 0.3764 | C | G |
| AX-40473767 | 0.006864808 | Clinical Relapse | 0.3819 | G | C |
| AX-13455223 | 0.006864808 | Clinical Relapse | 0.3819 | T | C |
| AX-40473637 | 0.007304168 | Clinical Relapse | 0.2238 | G | A |
| AX-40473755 | 0.010503394 | Clinical Relapse | 0.4341 | A | C |
| AX-11234805 | 0.01597391 | Clinical Relapse | 0.1264 | T | C |
| AX-32791879 | 0.021889851 | Clinical Relapse | 0.2747 | C | T |
| AX-32794831 | 0.02288708 | Clinical Relapse | 0.2155 | T | C |
| AX-11442739 | 0.022922586 | Clinical Relapse | 0.3516 | T | C |
| AX-32793975 | 0.02328333 | Clinical Relapse | 0.2225 | C | T |
| AX-94348052 | 0.024360008 | Clinical Relapse | 0.4171 | A | G |
| AX-32792425 | 0.03099842 | Clinical Relapse | 0.3956 | G | A |
| AX-40474339 | 0.031723819 | Clinical Relapse | 0.3654 | A | G |
| AX-11280328 | 0.031723819 | Clinical Relapse | 0.3654 | G | A |
| AX-32795157 | 0.034010136 | Clinical Relapse | 0.467 | T | C |
| AX-40473651 | 0.037110016 | Clinical Relapse | 0.2308 | A | G |
| AX-11234761 | 0.038636205 | Clinical Relapse | 0.1566 | C | T |
| AX-40474129 | 0.041219535 | Clinical Relapse | 0.3462 | C | T |
| AX-40474859 | 0.041637332 | Clinical Relapse | 0.2335 | A | G |
| AX-32794851 | 0.045745286 | Clinical Relapse | 0.228 | C | T |
| AX-13455842 | 0.049245636 | Clinical Relapse | 0.4669 | C | G |
| AX-13455332 | 0.049943394 | Clinical Relapse | 0.2225 | T | C |

HLA evolutionary diversity association with onset of relapse and sustained clinical response: High HLA Evolutionary Diversity in class I (on average but also for each of the A, B and C loci independently) and in class II region (on average but also for each of the DQB1, DPB1, DRB1 loci independently) were associated with (protective against) early onset of virological relapse.

High HLA Evolutionary Diversity in class I and class II regions on average was associated with sustained clinical response, and for HLA-C and HLA-DRB1 locus alone.

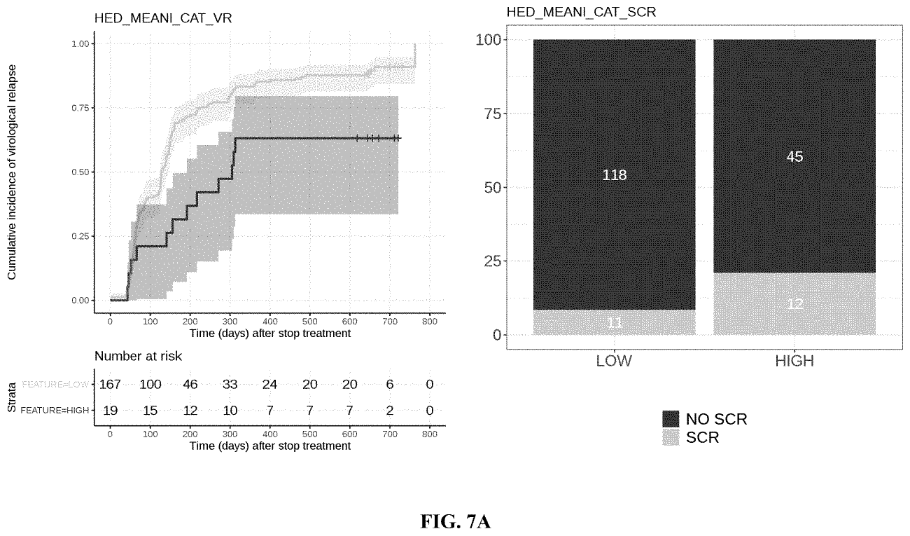

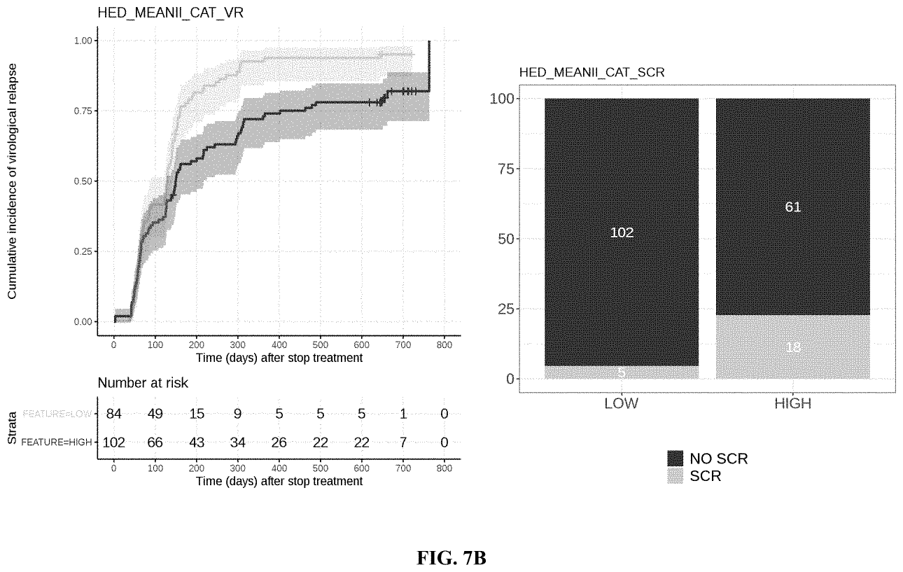

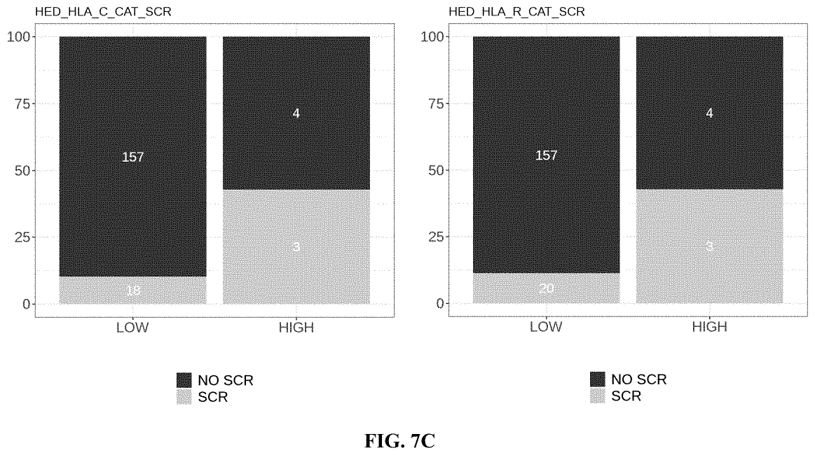

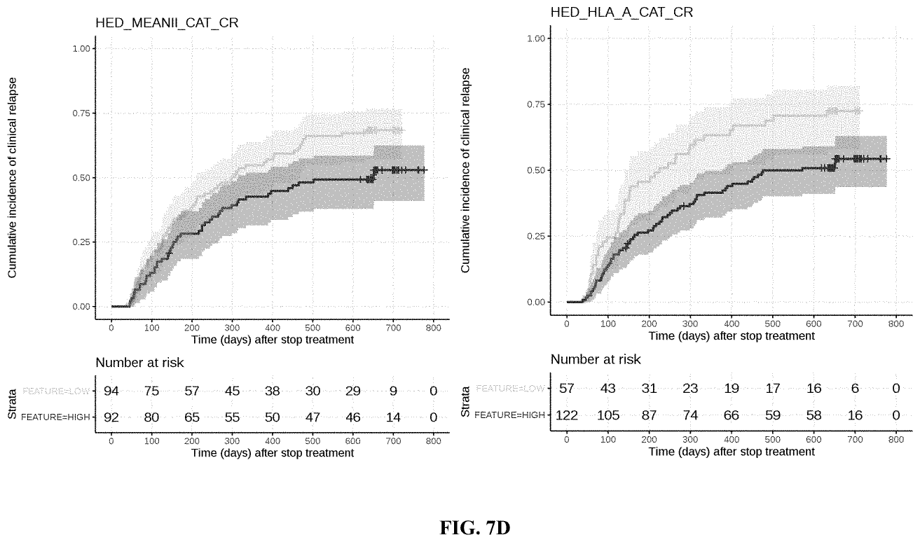

High HLA Evolutionary Diversity in class II on average and on HLA-A locus were protective against early onset of clinical relapse. All results are summarized on Table 5 and FIG. 7. Table 5 presents an overview of the HLA HED regions significantly (FDR<10%) associated with early onset of virological relapse, early onset of clinical relapse and sustained clinical response. For each locus or region, its estimate in the corresponding model is reported together with the unadjusted p-value and false discovery rate. The protective factor is indicated (high evolutionary diversity).

TABLE 5

| ANALYSIS | FEATURE | estimate | p. value | FDR | PROTECTIVE |
|---|---|---|---|---|---|
| CR | HED_MEANII_CAT_CR | −0.63 | 0.00 | 0.04 | high |
| CR | HED_HLA_A_CAT_CR | −0.56 | 0.01 | 0.09 | high |
| SCR | HED_MEANII_CAT_SCR | 1.91 | 0.00 | 0.02 | high |
| SCR | HED_HLA_C_CAT_SCR | 2.63 | 0.00 | 0.03 | high |
| SCR | HED_HLA_R_CAT_SCR | 2.37 | 0.00 | 0.04 | high |
| SCR | HED_MEANI_CAT_SCR | 1.19 | 0.01 | 0.08 | high |
| VR | HED_MEANII_CAT_VR | −0.66 | 0.00 | 0.00 | high |
| VR | HED_MEANI_CAT_VR | −0.79 | 0.01 | 0.07 | high |
| VR | HED_HLA_A_CAT_VR | −0.43 | 0.01 | 0.07 | high |
| VR | HED_HLA_Q_CAT_VR | −0.40 | 0.02 | 0.07 | high |
| VR | HED_HLA_B_CAT_VR | −0.43 | 0.02 | 0.07 | high |
| VR | HED_HLA_C_CAT_VR | −0.51 | 0.02 | 0.07 | high |
| VR | HED_HLA_P_CAT_VR | −0.35 | 0.03 | 0.07 | high |
| VR | HED_HLA_R_CAT_VR | −0.41 | 0.04 | 0.08 | high |

As indicated in FIGS. 7A-C, HLA evolutionary diversity (high vs. low applying the cut-off defined on Table 3) is significantly associated with onset of virological relapse, onset of clinical relapse and sustained clinical response. Kaplan-Meier curves representing HED scores for HLA class I and class II associated with onset of virological relapse are shown in FIGS. 7A and 7B. Barplots representing the distribution association of high HED score with sustained clinical responders are added to FIGS. 7A and 7B (mean class I and mean class II) and FIG. 7C (HLA-C and HLA-DRB1). Kaplan-Meier curves representing HED scores for HLA class II and HLA-A associated with onset of clinical relapse are shown in FIG. 7D.

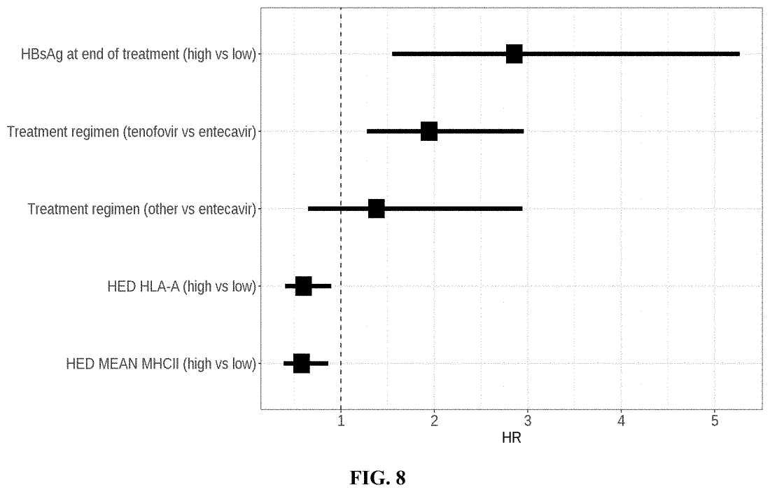

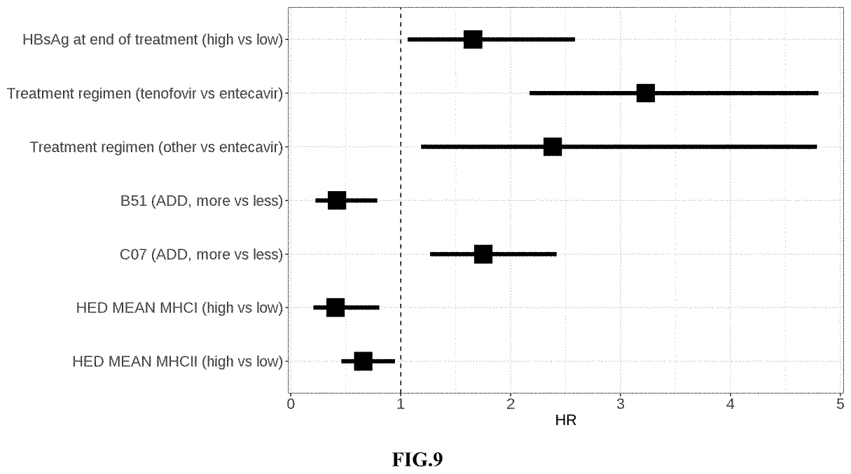

HLA signature associated with onset of clinical relapse: HB sAg at the last visit on treatment and treatment regimen were considered as main effects in the multivariate model. High HLA evolutionary diversity in both HLA-A and HLA class II regions are identified, by model comparison, as improving prediction of onset of clinical relapse in a multivariate model. Estimated hazard ratios are reported considering HBsAg level and treatment regimen as main effects in Table 7 and FIG. 8. Both Table 7 and FIG. 8 demonstrate Cox proportional hazard regression analysis for clinical relapse including HED score in HLA-A region and class II region (independently and together). For each variable, the estimated value of the hazard ratio (above/below 1) indicates the negative/positive association to relapse.

Performance of the multivariate models not including the HLA HED scores, including HED score in HLA-A region and HLA class II regions (separately and together) are compared by the AUC of the ROC curves predicting onset of clinical relapse (see Table 6). Table 6 presents the comparison of performances between a multivariate model including HBsAg and regimen as main effects, adding HED score in HLA-A region and HLA-class II region independently or together to predict the onset of clinical relapse: AIC, BIC, concordance and ROC AUC.

A slight improvement is observed comparing the model including HLA HED scores for both regions to the model including only the clinical parameters. The AUC of the time-dependent ROC curves, assessed at two years after stop treatment, corresponding to the models including the HLA HED scores equal 0.70 compared to 0.67 for the model including end of treatment HBsAg and treatment regimen alone.

TABLE 6

| Model | AIC | BIC | Concordance | AUC |
|---|---|---|---|---|
| HBsAg + regimen | 838.42 | 845.96 | 0.63 | 0.67 |
| HBsAg + regimen + | 832.39 | 842.44 | 0.66 | 0.7 |
| HED_HLA_A_CAT_CR | | | | |
| HBsAg + regimen + | 835.14 | 845.18 | 0.65 | 0.69 |
| HED_MEANII_CAT_CR | | | | |
| HBsAg + regimen + | 830.31 | 842.87 | 0.67 | 0.7 |
| HED_HLA_A_CAT_CR + | | | | |
| HED_MEANII_CAT_CR | | | | |

TABLE 7

| Covariate | Variable | HR | Lower | Upper |
|---|---|---|---|---|
| HBsAg at end of treatment | HBSAG_CATHIGH | 2.86 | 1.55 | 5.27 |
| (high vs low) | | | | |
| Treatment regimen | LAST_PR_CMTRTTENOFOVIR | 1.94 | 1.28 | 2.96 |
| (tenofovir vs entecavir) | | | | |
| HED HLA-A (high vs low) | HED_HLA_A_CAT_CRHIGH | 0.60 | 0.40 | 0.90 |
| HED_MEAN_MHCII | HED_MEANII_CAT_CRHIGH | 0.58 | 0.39 | 0.87 |
| (high vs low) | | | | |

HLA signature associated with onset of virological relapse: HBsAg at the last visit on treatment and treatment regimen were considered as main effects in the multivariate mode.

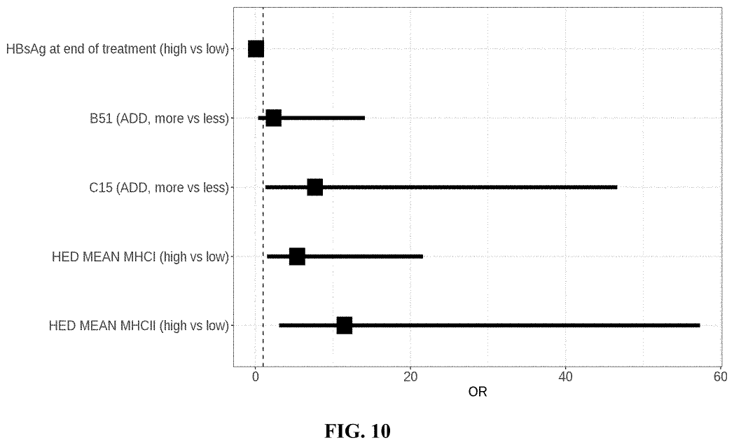

HLA-B*51 and HLA-C*07, together with high HLA evolutionary diversity in class I and class II regions are identified, by model comparison, as improving prediction of onset of virological relapse in a multivariate model. Estimated hazard ratios are reported considering HBsAg level and treatment regimen as main effects in Table 9 and FIG. 9. Both Table 9 and FIG. 9 demonstrate Cox proportional hazard regression analysis for virological relapse including HLA alleles B*51, HLA alleles C*07, HED scores in HLA class I and class II region. For each variable, the estimated value of the hazard ratio (above/below 1) indicates the negative/positive association to relapse.

Performance of the multivariate models including the HLA alleles, HLA HED scores (separately and together) are compared with a model without HLA information by the AUC of the ROC curves predicting onset of virological relapse (Table 8). Table 8 presents the comparison of performances between a multivariate model including HBsAg and regimen as main effects, adding HLA alleles B*51 and C*07, HED score in HLA-class I and HLA-class II regions independently or together to predict the onset of virological relapse: AIC, BIC, concordance and ROC AUC.

A clear improvement is observed comparing the model including HLA HED scores and HLA alleles to the model including only the clinical parameters. The AUC of the time-dependent ROC curves, assessed at two years after stop treatment, corresponding to the models including the HLA HED scores and HLA alleles equal 0.79 compared to 0.66 for the model including end of treatment HBsAg and treatment regimen alone.

TABLE 8

| Model | AIC | BIC | Concordance | AUC |
|---|---|---|---|---|
| HBsAg + regimen | 1188.76 | 1197.57 | 0.67 | 0.66 |
| HBsAg + regimen + | 1167.99 | 1182.66 | 0.72 | 0.77 |
| B*51_COUNT + | | | | |
| C*07_COUNT | | | | |
| HBsAg + regimen + | 1174.82 | 1189.49 | 0.71 | 0.73 |
| HED_MEANI_CAT_VR + | | | | |
| HED_MEANII_CAT_VR | | | | |
| HBsAg + regimen + | 1158.27 | 1178.81 | 0.73 | 0.79 |
| B*51_COUNT + | | | | |
| C*07_COUNT + | | | | |
| HED_MEANI_CAT_VR + | | | | |
| HED_MEANII_CAT_VR | | | | |

TABLE 9

| Covariate | Variable | HR | Lower | Upper |
|---|---|---|---|---|
| HBsAg at end of treatment | HBSAG_CATHIGH | 1.66 | 1.06 | 2.59 |
| (high vs low) | | | | |
| Treatment regimen | LAST_PR_CMTRTTENOFOVIR | 3.23 | 2.17 | 4.80 |
| (tenofovir vs entecavir) | | | | |
| Treatment regimen | LAST_PR_CMTRTOTHER | 2.38 | 1.19 | 4.79 |
| (other vs entecavir) | | | | |
| B51 (ADD, more vs less) | B51_COUNT | 0.42 | 0.22 | 0.79 |
| C07 (ADD, more vs less) | C07_COUNT | 1.75 | 1.27 | 2.42 |
| HED MEAN MHCI | HED_MEANI_CAT_VRHIGH | 0.41 | 0.20 | 0.81 |
| (high vs low) | | | | |
| HED MEAN MHCII | HED_MEANII_CAT_VRHIGH | 0.66 | 0.46 | 0.95 |
| (high vs low) | | | | |

HLA signature associated with sustained clinical response: HBsAg at the last visit on treatment was considered as main effect in the multivariate model. A clear improvement is observed comparing the model including HLA HED scores and HLA alleles to the model including only the clinical parameters. The ROC AUC values corresponding to the models including the HLA HED scores and HLA alleles equal 0.91 compared to 0.67 for the model including only end of treatment HBsAg (Table 10). Table 10 presents the comparison of performances between a multivariate model including HBsAg as main effect, adding HLA alleles B*51 and C*15 counts, adding HED score in HLA-class I and HLA-class II regions and HED HLA-C and HED HLA-DRB1 independently or together, or adding HED HLA-class I and HED HLA-class II only to predict sustained clinical response: AIC, BIC, and ROC AUC.

TABLE 10

| Model | AIC | BIC | AUC |
|---|---|---|---|
| HBsAg | 111.58 | 117.73 | 0.67 |
| HBsAg + B51_COUNT + | 103.44 | 115.74 | 0.79 |
| C15_COUNT | | | |
| HBsAg + | 89.66 | 108.11 | 0.87 |
| HED_MEANI_CAT_SCR + | | | |
| HED_HLA_C_CAT_SCR + | | | |
| HED_MEANII_CAT_SCR + | | | |
| HED_HLA_R_CAT_SCR | | | |
| HBsAg + B51_COUNT + | 87.24 | 111.84 | 0.91 |
| C15_COUNT + | | | |
| HED_MEANI_CAT_SCR + | | | |
| HED_HLA_C_CAT_SCR + | | | |
| HED_MEANII_CAT_SCR + | | | |
| HED_HLA_R_CAT_SCR | | | |
| HBsAg + B51_COUNT + | 89.95 | 108.41 | 0.89 |
| C15_COUNT + | | | |
| HED_MEANI_CAT_SCR + | | | |
| HED_MEANII_CAT_SCR | | | |

Interestingly, a model including HLA-B*51 and HLA-C*15 counts together with mean HED scores for class I and class II regions gave pretty similar predictive performance (BIC of 108.41 compared to BIC of 111.84 with the full model, for a slightly lower AUC, 0.89 compared to 0.91 with the full model). The output of this latest model is summarized on Table 11 and FIG. 10. Table 11 presents logistic regression analysis (estimated odds ratios and confidence intervals) for sustained clinical response including HLA alleles B*51, HLA alleles C*51, HED scores in HLA class I and class II region. FIG. 10 demonstrates logistic regression analysis for sustained clinical response including HLA alleles B*51, HLA alleles C*51, HED scores in HLA class I and class II region. For each variable, the estimated value of the odds ratio (below/above 1) indicates the negative/positive association to sustained clinical response.

TABLE 11

| Covariate | Variable | OR | Lower | Upper |
|---|---|---|---|---|
| HBsAg at end of treatment | HBSAG_CATHIGH | 0.09 | 0.02 | 0.33 |
| (high vs low) | | | | |
| B51 (ADD, more vs less) | B51_COUNT | 2.34 | 0.36 | 14.12 |
| C15 (ADD, more vs less) | C15_COUNT | 7.68 | 1.29 | 46.66 |
| HED MEAN MHCI | HED_MEANI_CAT_SCRHIGH | 5.38 | 1.51 | 21.62 |
| (high vs low) | | | | |
| HED MEAN MHCII | HED_MEANII_CAT_SCRHIGH | 11.49 | 3.08 | 57.35 |
| (high vs low) | | | | |

Discussion: In this large prospective stop study, several HLA class I alleles (or SNPs in HLA-C region as shown in FIG. 6D, showing the same level of significance) were shown to be predictive of late onset of virological relapse and sustained clinical response. Most of the studies exploring a link between HLA alleles and chronic hepatitis B disease focused on HLA class II region (Wang, et al, Journal of Immunology Research, 2016, DOI:10.1155/2016/9069375; Singh, World Journal of Gastroenterology, 2007, 13: 1770) and association with susceptibility to chronic infection and disease progression. If an association has been reported between SNPs in HLA-C region (together with KIR region) and response to IFN treatment (Khakoo, Science 2004, 305: 872-4), this is to our knowledge, the first time that HLA class I region has been described to play a role in long term response (up to two years without virological relapse) to NUC therapy. The mechanisms by which HLA class I play a role in CHB in long term antiviral treatment response is still unknown but both have been reported to play a role in control of other chronic infections such as in HCV (Khakoo, Science 2004, 305: 872-4) and HIV (Science 2010; 330: 1551-7), by interacting with Natural Killer cells and/or by peptide presentation to CD8 positive T cells. In this cohort, punctual mutations in genes coding for LILR ligands and KIR ligands interacting with HLA-B and HLA-C proteins could be observed, suggesting a potential role of antigen presenting cells and their interaction with Natural Killer cells in long term response. It would be important to replicate those observations in independent cohort, and explore further the functional consequences of those genetic variations

Next to this major role of HLA class I alleles in long term treatment response, in this unique cohort high HLA Evolutionary Diversity in both class I and class II regions were shown to be protective against early onset of virological relapse and predictive of sustained clinical response. HLA allelic heterozygosity was not significantly associated with onset of relapse in this study, while larger functional diversity in the HLA class I and class II seems to be predictive of long term response to direct antiviral treatment.

The importance of HED (especially in class I region) has been described earlier in response to immunotherapy in cancer (Chowell, et al., Science, 2018, 359: 582-7; Chowell, et al., Nature Medicine, 2019, 25: 1715-20) and in control of HIV infection (Arora, et al., Molecular Biology and Evolution, 2020, 37: 639-50; Carrington, et al., Science 1999, 283: 1748; Arora, et al., Proceedings of the National Academy of Sciences of the United States of America 2019, 116: 944-9), but has not been explored in antiviral treatment response to chronic HBV infection.

Both aspects (allelic advantage and overall HLA functional diversity) seem to contribute significantly to predict antiviral treatment response and can be measured with one assay (HLA typing), This HLA signature can be a useful contributor in predicting patient outcome when considering stopping suppression treatment.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the present description.

All documents cited herein are incorporated by reference.

1. An in vitro method of determining whether a subject having a chronic hepatitis B (CHB) infection has a high or low probability of relapse after discontinuation of a viral suppression treatment, comprising:
a. detecting in a biological sample obtained from the subject the presence of one or more immunogenetic biomarkers of viral control of the CHB infection, wherein the one or more immunogenetic biomarkers are selected from the group consisting of:
i. a human leukocyte antigen (HLA) allele;
ii. one or more HLA-C single nucleotide polymorphisms (SNPs);
iii. a HLA evolutionary diversity (HED) score; and
iv. leucocyte receptor complex (LCR) SNPs;
b. determining that the probability of relapse is low for the subject if the one or more immunogenetic biomarkers are detected in the biological sample, or determining that the probability of relapse is high for the subject if none of the immunogenetic biomarkers is detected in the biological sample.

2. A method of treating a chronic hepatitis B (CHB) infection in a subject in need thereof, comprising:
a. administering to the subject a viral suppression treatment to treat the CHB infection;
b. detecting in a biological sample obtained from the subject the presence of one or more immunogenetic biomarkers of viral control of the CHB infection, wherein the one or more immunogenetic biomarkers are selected from the group consisting of:
i. a human leukocyte antigen (HLA) allele;
ii. one or more HLA-C single nucleotide polymorphisms (SNPs);
iii. a HLA evolutionary diversity (HED) score; and
iv. leucocyte receptor complex (LCR) SNPs;
c. if the one or more immunogenetic biomarkers of step b are detected in the biological sample, discontinuing the viral suppression treatment once viral suppression is achieved; or if none of the immunogenetic biomarkers of step b is detected in the biological sample, continuing the viral suppression treatment even after viral suppression is achieved, and/or administering to the subject a further or different viral suppression treatment.

3. A method of treating a chronic hepatitis B (CHB) infection in a subject in need thereof, the method comprising:
a. administering to the subject a viral suppression treatment to treat the CHB infection;
b. detecting in a biological sample obtained from the subject the presence of one or more immunogenetic biomarkers of viral control of the CHB infection, wherein the one or more immunogenetic biomarkers are selected from the group consisting of:
i. a human leukocyte antigen (HLA) allele;
ii. one or more HLA-C single nucleotide polymorphisms (SNPs);
iii. a HLA evolutionary diversity (HED) score; and
iv. leucocyte receptor complex (LCR) SNPs;
c. discontinuing the viral suppression treatment when the CHB infection is suppressed in the subject, and
d. administering to the subject the viral suppression treatment or another viral suppression treatment less than two years after the discontinuation if none of the immunogenetic biomarkers is detected in the biological sample.

4. The method of claim 1, wherein the immunogenetic biomarker comprises the presence of an HLA allele of B*51 or C*15, or the absence of an HLA allele C*07.

5. The method of claim 1, wherein the immunogenetic biomarker comprises an HLA-C SNP selected from the group consisting of rs2394952, rs3130542, rs2894202, rs9264523, rs1049281, rs9264643, rs1130838, rs2394888, AX-83089411, rs2308622, rs9264416, rs2001181, rs3132499, rs3130532, rs3130941, rs3130528, rs3134782, rs3134769, rs3130521, rs3130695, rs3130685, rs2524119, rs3130527, rs2894186, rs3130439, rs3095254, rs9264189, rs2394943, rs9394047, rs3130948, rs9368666, rs3130942, rs3130688, rs3130536, rs3134750, rs4084090, rs9264127, rs9264039, and rs3868078, or a complementary sequence thereof.

6. The method of claim 1, wherein the immunogenetic biomarker comprises at least one HED score larger than the threshold value selected from the table below:

| HLA Regions | HLA gene | Variable | Threshold |
|---|---|---|---|
| Mean HLA Class I | HLA-A | HED_HLA_A_CAT_VR | 3.88 |
| Mean HLA Class I | HLA-B | HED_HLA_B_CAT_VR | 9.37 |
| Mean HLA Class I | HLA-C | HED_HLA_C_CAT_VR | 0.00 |
| Mean HLA Class I | MEAN_MHCI | HED_MEANI_CAT_VR | 8.69 |
| Mean HLA Class II | HLA-DPB1 | HED_HLA_P_CAT_VR | 2.21 |
| Mean HLA Class II | HLA-DQB1 | HED_HLA_Q_CAT_VR | 11.98 |
| Mean HLA Class II | HLA-DRB1 | HED_HLA_R_CAT_VR | 12.68 |
| Mean HLA Class II | MEAN_MHCII | HED_MEANII_CAT_VR | 7.55 |
| Mean HLA Class I | HLA-A | HED_HLA_A_CAT_CR | 3.88 |
| Mean HLA Class II | MEAN_MHCII | HED_MEANII_CAT_CR | 7.67 |
| Mean HLA Class I | HLA-C | HED_HLA_C_CAT_SCR | 7.37 |
| Mean HLA Class I | MEAN_MHCI | HED_MEANI_CAT_SCR | 7.61 |
| Mean HLA Class II | HLA-DRB1 | HED_HLA_R_CAT_SCR | 14.16 |
| Mean HLA Class II | MEAN_MHCII | HED_MEANII_CAT_SCR | 8.21. |

7. The method of claim 1, wherein the immunogenetic biomarker comprises a LCR SNP selected from the group consisting of rs10426302, rs59537494, rs28366008, rs36625, rs635608, rs7595, rs731170, rs28513, rs12459334, rs11666535, rs4806807, rs11084367, rs39714, rs1654474, rs12462907, rs775893, rs10416527, rs4441391, rs40167, rs11084339, rs775875, rs2304225, rs4077076, rs4442924, rs4806527, rs12608979, rs3765013 of COSV52557220, rs12462181, rs2075731 of COSV52550169, rs12608988, rs190480734, rs1654452, rs11879415, rs653560, rs11084387, rs11672111, rs10424969, rs77389424, rs3745902 of CM1111041, rs11672983, rs17836364, rs34549987, rs11668526, rs11667105, rs1749282, rs1654660, rs73618328, rs270785, rs76522818, rs62131745, rs2241384, rs1325158, rs10500318, rs3816051, rs34508934, rs12460627, rs11667812, rs12974194, rs17836364, rs11669431, rs12984962, rs1761462, rs4806464, rs34190750, rs28681595, rs12610675, rs12463051, rs1749282, rs1654660, rs41275824, rs2241384, rs12983338, rs272408, rs10412569, AX-3232794851, rs622941, and rs60690598, or a complementary sequence thereof.

8. The method of claim 1, further comprising measuring the level of at least one of HBV DNA, alanine aminotransferase (ALT), and hepatitis B e-antigen (HBeAg) in a biological sample of the subject.

9. The method of claim 1, wherein the viral suppression treatment comprises administering to the subject a therapeutically effective amount of a nucleotide or nucleoside analogue (NUC).

10. The method of claim 1, wherein the one or more HLA-C SNPs are selected from the group consisting of rs2394952, rs3130542, rs2894202, rs9264523, rs1049281, rs9264643, rs1130838, rs2394888, AX-83089411, rs2308622, rs9264416, rs2001181, rs3132499, rs3130532, rs3130941, rs3130528, rs3134782, rs3134769, rs3130521, rs3130695, rs3130685, rs2524119, rs3130527, rs2894186, rs3130439, rs3095254, rs9264189, rs2394943, rs9394047, rs3130948, rs9368666, rs3130942, rs3130688, rs3130536, and rs3134750, or a complementary sequence thereof.

11. The method of claim 1, wherein the one or more HLA-C SNPs are selected from the group consisting of rs4084090, rs9264127, rs9264039, and rs3868078, or a complementary sequence thereof.

12. The method of claim 1, wherein the HED score is selected from the group consisting of a value of HED HLA-A larger than 3.88, a value of HED HLA-B larger than 9.37, a value of HED HLA-C larger than 0.00, a value of mean HED HLA Class I larger than 8.69, a value of HED HLA-DPB1 larger than 2.21, a value of HED HLA-DQB1 larger than 11.98, a value of HED HLA-DRB1 larger than 12.68, and a value of mean HED HLA Class II larger than 7.55.

13. The method of claim 1, wherein the HED score is selected from the group consisting of a value of HED HLA-A larger than 3.88, and a value of mean HED HLA Class II larger than 7.67.

14. The method of claim 1, wherein the HED score is selected from the group consisting of a value of HED HLA-C larger than 7.37, a value of mean HED HLA Class I larger than 7.61, a value of HED HLA-DRB1 larger than 14.16, and a value of mean HED HLA Class II larger than 8.21.

15. The method of claim 1, wherein the one or more LCR SNPs are selected from the group consisting of rs10426302, rs59537494, rs28366008, rs36625, rs635608, rs7595, rs731170, rs28513, rs12459334, rs11666535, rs4806807, rs11084367, rs39714, rs1654474, rs12462907, rs775893, rs10416527, rs4441391, rs40167, rs11084339, rs775875, rs2304225, rs4077076, rs4442924, rs4806527, rs12608979, rs3765013 of COSV52557220, rs12462181, rs2075731 of COSV52550169, rs12608988, rs190480734, rs1654452, rs11879415, rs653560, rs11084387, rs11672111, rs10424969, rs77389424, rs3745902 of CM1111041, rs11672983, rs17836364, rs34549987, rs11668526, rs11667105, rs1749282, rs1654660, rs73618328, rs270785, rs76522818, rs62131745, rs2241384, rs1325158, rs10500318, rs3816051, rs34508934, and rs12460627, or a complementary sequence thereof.

16. The method of claim 1, wherein the one or more LCR SNPs are selected from the group consisting of rs11667812, rs12974194, rs17836364, rs11669431, rs12984962, rs1761462, rs4806464, rs34190750, rs28681595, rs12610675, rs12463051, rs1749282, rs1654660, rs41275824, rs2241384, rs12983338, rs272408, rs10412569, AX-3232794851, rs622941, and rs60690598, or a complementary sequence thereof.

17. The method of claim 1, wherein the SNP or the allele is determined by a method selected from the group consisting of DNA sequencing, restriction fragment length polymorphism (RFLP analysis), allele specific oligonucleotide (ASO) analysis, Denaturing/Temperature Gradient Gel Electrophoresis (DGGE/TGGE), Single-Strand Conformation Polymorphism (SSCP) analysis, Dideoxy fingerprinting (ddF), pyrosequencing analysis, acycloprime analysis, Reverse dot blot, GeneChip microarrays, Dynamic allele-specific hybridization (DASH), Peptide nucleic acid (PNA) and locked nucleic acids (LNA) probes, TaqMan, Molecular Beacons, Intercalating dye, FRET primers, AlphaScreen, SNPstream, genetic bit analysis (GBA), Multiplex minisequencing, SNaPshot, MassEXTEND, MassArray, GOOD assay, Microarray miniseq, arrayed primer extension (APEX), Microarray primer extension, Tag arrays, Coded microspheres, Template-directed incorporation (TDI), fluorescence polarization, Colorimetric oligonucleotide ligation assay (OLA), Sequence-coded OLA, Microarray ligation, Ligase chain reaction, Padlock probes, Rolling circle amplification, and Invader assay.

18. The method of claim 1, wherein the viral suppression agent is a nucleotide or nucleoside analogue (NUC), and the NUC is selected from the group consisting of tenofovir, entecavir, lamivudine, adefovir, and telbivudine.

19. The method of claim 1, wherein the subject discontinues the viral suppression treatment when the subject achieves at least one of HBV DNA <60 IU/mL, alanine aminotransferase (ALT) <80 U/L, and hepatitis B e-antigen (HBeAg) negative.

20. The method of claim 19, wherein the subject further achieves HBsAg <100 IU/mL at the time of discontinuation of the viral suppression treatment.

21. The method of claim 1, wherein the subject has no virological relapse at or after 3 months, 6 months, 12 months, 18 months, 24 months, or 36 months after the discontinuation of the viral suppression treatment, or anytime in between, and the virological relapse is identified as HBV DNA ≥2000 IU/ml or HBeAg positive.

22. The method of claim 1, wherein the subject has no clinical relapse at or after 3 months, 6 months, 12 months, 18 months, 24 months, or 36 months after the discontinuation of the viral suppression treatment, or anytime in between, and the clinical relapse is identified as i) HBV DNA ≥2000 IU/ml or HBeAg positive, and ii) ALT ≥80 U/L.

23. The method of claim 1, wherein the biological sample is a tissue sample, a cellular sample, or a blood sample.

24. A combination for predicting low risk of relapse after viral suppression in the treatment of a chronic hepatitis B (CHB) infection in a subject in need thereof, the combination comprising a reagent capable of detecting the one or more of the immunogenetic biomarkers of claim 1.

25. The combination of claim 24, further comprising one or more therapeutic agents for treating the CHB.

26. A kit for serological HLA typing, or a kit for genetic HLA typing, for use in predicting efficacy of a viral suppression agent in treating a chronic hepatitis B (CHB) infection in a subject in need thereof.

27. (canceled)
